(19) United States
(12) Patent Application Publication
(10) Pub. No.: US 2024/0428918 A1
(43) Pub. Date: Dec. 26, 2024

(54) CUSTOM MEDICAMENT INJECTION SYSTEMS, METHODS, AND INJECTORS

(71) Applicant: PROMETHEOS, INC., WILMINGTON, DE (US)

(72) Inventor: Spero J.V. THEODOROU, Miami, FL (US)

(21) Appl. No.: 18/632,939

(22) Filed: Apr. 11, 2024

Related U.S. Application Data
(60) Provisional application No. 63/522,494, filed on Jun. 22, 2023.

(51) Int. Cl. G16H 20/17; A61M 5/19; A61M 5/20; A61M 5/30; A61M 5/315; G16H 10/60

(52) U.S. Cl. CPC G16H 20/17; A61M 5/19; A61M 5/2066; A61M 5/30; A61M 5/31535; A61M 5/31578; A61M 5/31593; A61M 5/31596; G16H 10/60; A61M 2005/2073; A61M 2202/0007; A61M 2202/07; A61M 2205/10; A61M 2205/121; A61M 2205/3306; A61M 2205/3327; A61M 2205/3379; A61M 2205/3569; A61M 2205/3592; A61M 2205/505; A61M 2205/583; A61M 2205/8206; A61M 2209/00; A61M 2210/04

(57) ABSTRACT

Custom medicament injection systems and methods suitable for injection of a medicament into at least one treatment area in the skin of a user may include a medicament injection component and a computing device. The medicament injector component may be configured to discharge a selected quantity, volume, and/or number of injection dosage units of medicament from a medicament cartridge as a high-pressure jet of the medicament into the patient's skin. The computing device may be configured to capture pre-treatment data (e.g. an image of the treatment area on the patient) and, based on the pre-treatment data, identify and output targeting data including one or more target medicament injection sites relative to the treatment area.

### CROSS-REFERENCE TO RELATED APPLICATION

This patent application claims priority to and the benefit of the filing date of U.S. provisional patent application No. 63/522,494 filed Jun. 22, 2023 for Medicament Injection Systems, Methods, and Injectors, the entire contents of which is hereby incorporated by reference.

### FIELD

Illustrative embodiments of the disclosure generally relate to systems, methods, and devices suitable for custom epidermal, dermal, subdermal, subcutaneous, or intramuscular injection of medical substances into a patient. More particularly, illustrative embodiments of the disclosure relate to medicament injection systems, methods, and injectors which may be suitable for patient self-injection of medicaments which may include but are not limited to botulinum toxin into or beneath the skin at one or more treatment areas on the patient.

### SUMMARY

Illustrative examples of the disclosure are generally directed to medicament injection systems and methods suitable for custom injection of a medicament into at least one treatment area on a user.

### Example 1

In a first example a custom medicament injection system includes a medicament injection component and a computing device. The medicament injection component includes an injector and a replaceable medicament cartridge. Actuation of the injector injects a set medicament dosage into or beneath a person's skin. The computing device includes a processor device and a non-transitory computer-readable medium having instructions stored thereon that are executable by the processor device to cause the computing device to: (1) receive pre-treatment data about a treatment area associated with the person's skin; (2) based on the pre-treatment data about the treatment area, identify one or more target medicament injection sites relative to the treatment area; and (3) output targeting data for the one or more target medicament injection sites relative to the treatment area.

In some implementations, identifying the one or more target medicament injection sites relative to the treatment area constitutes identifying at least one muscle animation associated with the person's skin.

In some implementations the pre-treatment data is at least one captured image of the treatment area including at least one indicia associated with at least one muscle animation.

In some implementations the at least one captured image is a static image or a video image.

In some implementations the at least one indicia is at least one rhytid in the treatment area.

In some implementations the non-transitory computer-readable medium has instructions stored thereon that are executable by the processor device to cause the computing device to, based on a spatial relationship between the at least one rhytid and an animation of at least one muscle associated with the at least one rhytid, identify the one or more target medicament injection sites relative to the treatment area.

In some implementations the computing device component also includes a camera that captures the at least one image.

In some implementations the system also includes a display.

In some implementations the display outputs the targeting data.

In some implementations the display outputs the targeting data overlaid on an image including the treatment area.

In some implementations the computing device includes the display.

In some implementations the non-transitory computer-readable medium has instructions stored thereon that are executable by the processor device to cause the computing device to identify one or more target medicament dosages for the one or more target medicament injection sites.

In some implementations the non-transitory computer-readable medium has instructions stored thereon that are executable by the processor device to cause the computing device to receive post-treatment data about the treatment area associated with the person's skin.

In some implementations the non-transitory computer-readable medium has instructions stored thereon that are executable by the processor device to cause the computing device to, based on the post-treatment data, output a recommendation for a retreatment.

In some implementations the retreatment includes at least one modified target medicament injection site or at least one modified injection dosage.

In some implementations the non-transitory computer-readable medium has instructions stored thereon that are executable by the processor device to cause the computing device to receive follow-up data about the treatment area associated with the person's skin.

In some implementations the non-transitory computer-readable medium has instructions stored thereon that are executable by the processor device to cause the computing device to retain data about earlier treatments.

In some implementations the non-transitory computer-readable medium has instructions stored thereon that are executable by the processor device to cause the computing device to, based on the data about earlier treatments and based on the follow-up data, output a recommendation for a follow-up treatment.

In some implementations the follow-up treatment includes at least one modified target medicament injection site or at least one modified injection dosage.

### Example 2

In a second example a method of administering a botulinum toxin treatment includes a step of receiving a medicament cartridge from a supplier, the medicament cartridge including a constituted botulinum toxin. The method further includes installing the medicament cartridge in a medicament injection device, in which the medicament injection device and medicament cartridge are part of a medicament injection system configured such that, when the medicament cartridge is installed in the medicament injection device, actuation of the medicament injection device injects a pre-determined volume of the constituted botulinum toxin from the medicament cartridge. The method further includes positioning the medicament injection device on or adjacent a person's skin and actuating the medicament injection device to inject the pre-determined volume of liquid from the medicament cartridge into the person.

In some implementations the method of administering the botulinum toxin treatment is a method of self-administering the botulinum toxin treatment.

In some implementations the constituted botulinum toxin comprises a ratio in the range of 1 to 200 units of botulinum toxin to 0.1 mL to 0.2 mL of diluent.

In some implementations the diluent is a sodium chloride solution.

In some implementations the cartridge includes a liquid volume of the botulinum toxin and diluent of between 1 and 10 mL.

In some implementations the medicament cartridge is received in a sealed and ambient temperature state.

In some implementations the medicament cartridge includes an internal plunger.

In some implementations the medicament cartridge includes an injection tip or includes a nozzle configured to connect to an injection tip of the medicament injection device.

In some implementations the medicament cartridge has an outer housing, and the medicament cartridge device has a medicament receptacle dimensioned and configured to receive at least a portion of and connect to the outer housing of the medicament cartridge such that the medicament injection device can be actuated to inject the pre-determined volume of liquid from the medicament cartridge.

In some implementations the method further includes, prior to receiving the medicament cartridge, using a computing device of a custom medicament injection system to receive pre-treatment data about a treatment area associated with the person's skin, and, based on the pre-treatment data about the treatment area, determine that a treatment is indicated.

In some implementations the method further includes, based on the treatment indication, transmitting an order for the medicament cartridge from the supplier.

### Example 3

In a third example a medicament injection device includes a medicament injector, an injector actuator, and a liquid supply. The injector actuator operates the medicament injector to inject a pre-determined volume of a medicament from the medicament injection device, the medicament drawn from a medicament cartridge removably installed in the medicament injection device. The liquid supply is in fluid communication with the medicament cartridge when the medicament cartridge is removably installed in the medicament injection device. The medicament injection device mixes liquid from the liquid supply with the medicament from the medicament cartridge prior to injection.

In some implementations the medicament injection device flows a pre-determined volume of liquid through the liquid supply into the medicament cartridge removably installed in the medicament injection device.

In some implementations the liquid supply flows the pre-determined volume of liquid into the medicament cartridge from a liquid supply cartridge removably installed in the medicament injection device.

In some implementations the medicament cartridge is a vial of botulinum toxin and the liquid supply cartridge is a vial of sodium chloride solution.

In some implementations the medicament injection device includes a first port with a first piercing tip for piercing a stopper of the medicament cartridge and a second port with a second piercing tip for piercing a stopper of the liquid supply cartridge when the medicament and liquid supply cartridges are removably installed in the medicament injection device.

In some implementations the first and second ports are replaceable components of the medicament injection device.

### Example 4

As a fourth example a method of administering a medicament treatment uses a medicament injection device including a medicament injector and an injection actuator that operates the medicament injector to inject a pre-determined volume of a medicament from the medicament injection device. The method includes a step of installing a medicament cartridge into the medicament injection device, the medicament cartridge including a medicament. The method further includes a step of after the medicament cartridge is installed into the medicament injection device, supplying a liquid into the medicament cartridge to reconstitute the medicament. The method further includes a step of after reconstituting the medicament, operating the injection actuator to inject a pre-determined volume of the reconstituted medicament from the medicament injection device.

In some implementations the method also includes installing a liquid supply cartridge into the medicament injection device, the liquid supply cartridge including the liquid. Supplying the liquid into the medicament cartridge includes flowing the liquid from the liquid supply cartridge into the medicament cartridge.

In some implementations the medicament cartridge is a vial of botulinum toxin and the liquid supply cartridge is a vial of sodium chloride solution.

In some implementations the vials of botulinum toxin and sodium chloride solution are installed in the medicament injection device in a removable fashion.

In some implementations the medicament injection device flows a predetermined volume of sodium chloride solution into the botulinum toxin vial to reconstitute the botulinum toxin at a ratio in the range of 1 to 200 units of botulinum toxin to 0.1 mL to 0.2 mL of sodium chloride solution.

In some implementations the medicament cartridge is a vacuum sealed vial of botulinum toxin.

In some implementations the medicament cartridge is a vacuum sealed vial of dehydrated botulinum toxin.

### BRIEF DESCRIPTION OF THE DRAWINGS

Illustrative embodiments of the disclosure will now be described, by way of example, with reference to the accompanying drawings, in which:

FIG. 1 is a functional block diagram of one example of a custom medicament injection system;

FIG. 2 is a functional block diagram of a treatment management subsystem of the system of FIG. 1;

FIG. 3 is a functional block diagram of a medicament injector of the system of FIG. 1;

FIG. 4 is a functional block diagram of interfacing between a subsystem camera of the treatment management subsystem of FIG. 2 and an injector camera on the medicament injector of FIG. 3 with an external data processing/storage subsystem and access of a video data file stored in the external data processing/storage subsystem by a remote healthcare provider in some implementations of the medicament injection systems and methods;

FIG. 5 is a front perspective view of the medicament injector of the system of FIG. 1;

FIG. 6 is left side view of the medicament injector of FIG. 5;

FIG. 7 is a front view of the medicament injector of FIG. 5;

FIG. 8 is a bottom view of the medicament injector of FIG. 5;

FIG. 9 is an exploded front perspective view of the medicament injector of FIG. 5, more particularly illustrating an exemplary thread technique for attaching a medicament cartridge to a cartridge attachment nipple on the medicament injector;

FIG. 10 is a longitudinal sectional view of the medicament injector of FIG. 5, with a safety mechanism of the medicament injector deployed in a safety position;

FIG. 11 is a longitudinal sectional view of the medicament injector of FIG. 5, with the safety mechanism deployed in a primed position for use of the medicament injector;

FIG. 12 is a bottom front perspective view of the medicament injector of FIG. 5, with the bottom housing subunit removed from the upper housing subunit of the injector housing to expose various internal components of the medicament injector;

FIG. 13 is a bottom front perspective view of the medicament injector of FIG. 5, with the bottom housing subunit removed from the upper housing subunit of the injector housing;

FIG. 14 is a front perspective view of the medicament injector of FIG. 5 as the injector is positioned to inject medicament into the face of a patient, further illustrating presentation of an image of the patient's face as part of a facial injection map on a personal electronic device supported by a smart stand, which smart stand and personal electronic device form a part of the treatment management subsystem of FIG. 1;

FIG. 15 is a rear perspective view of the system of FIG. 14 as the medicament injector is positioned to inject the medicament into the patient's face;

FIG. 16 is a front perspective view of another example of a custom medicament injection system, with the image of the patient's face appearing as a reflected facial injection map on a smart mirror which forms a part of the treatment management subsystem;

FIG. 17 is a front perspective view of the smart stand and personal electronic device of the treatment management subsystem illustrated in FIGS. 14 and 15, further illustrating an injector holder on the smart stand and the injector supported by the injector holder;

FIG. 18 is a front perspective view of the smart stand of FIG. 17 with the personal electronic device and the medicament injector (not illustrated) removed from the smart stand;

FIG. 19 is a rear view of the smart stand with the personal electronic device and the medicament injector supported by the smart stand;

FIG. 20 is a front view of an example image of the face of a user as the image appears as a virtual facial injection map on the display of the treatment management subsystem, more particularly illustrating virtual injection targets appearing on the facial image for guiding injection of the medicament into the patient's face at the respective virtual injection targets;

FIG. 21 is a side view of the medicament injector of the system of FIG. 1 as the injector may initially be inadvertently positioned at an improper injection angle with respect to the plane of the skin on the face of the user upon initial placement of the injector against the skin preparatory to injection of the medicament, particularly in self-injection of the medicament by the user, and emission of an angle sensor beam from an angle sensor on the injector in detection of the improper injection angle;

FIG. 22 is a side view of the medicament injector illustrated in FIG. 21, after the position of the injector is corrected to the proper perpendicular injection angle;

FIG. 23 is a front perspective view of another example of a medicament injector, which in this example is gas-actuated;

FIG. 24 is a side view of the medicament injector of FIG. 23;

FIG. 25 is a front view of the medicament injector of FIG. 23;

FIG. 26 is an exploded front perspective view of the medicament injector of FIG. 23, with the main housing portion separated from the handle portion of the injector housing and the medicament cartridge removed from the cartridge housing;

FIG. 27 is an exploded front perspective view of the medicament injector of FIG. 23, with the main housing portion separated from the handle portion of the injector housing and the medicament cartridge (not illustrated) inserted in the cartridge housing;

FIG. 28 is a longitudinal sectional view of the medicament injector of FIG. 23, with a safety mechanism of the medicament injector deployed in a safety position;

FIG. 29 is a longitudinal sectional view of the medicament injector of FIG. 23, with the safety mechanism deployed in a primed position for use of the medicament injector;

FIG. 30 is a front perspective view of another example of a gas-actuated medicament injector;

FIG. 31 is a side view of the medicament injector of FIG. 30;

FIG. 32 is a front view of the medicament injector of FIG. 30;

FIG. 33 is an exploded front perspective view of the medicament injector of FIG. 30, with the main housing portion separated from the handle portion of the injector housing and the medicament cartridge removed from the cartridge housing;

FIG. 34 is an exploded front perspective view of the medicament injector of FIG. 30, with the main housing portion separated from the handle portion of the injector housing and the medicament cartridge (not illustrated) inserted in the cartridge housing;

FIG. 35 is a longitudinal sectional view of the medicament injector of FIG. 30, with a safety mechanism of the medicament injector deployed in a safety position;

FIG. 36 is a longitudinal sectional view of the medicament injector of FIG. 30, with the safety mechanism deployed in a primed position for use of the medicament injector;

FIG. 37 is a front perspective view of a spring-actuated example of a medicament injector;

FIG. 38 is a side view of the medicament injector of FIG. 37;

FIG. 39 is a front view of the medicament injector of FIG. 37;

FIG. 40 is an exploded front perspective view of the medicament injector of FIG. 37, with the main housing portion separated from the handle portion of the injector housing and the medicament cartridge removed from the cartridge housing;

FIG. 41 is an exploded front perspective view of the medicament injector of FIG. 37, with the main housing portion separated from the handle portion of the injector housing and the medicament cartridge (not illustrated) inserted in the cartridge housing;

FIG. 42 is a longitudinal sectional view of the medicament injector of FIG. 37, with a safety mechanism of the medicament injector deployed in a safety position;

FIG. 43 is a longitudinal sectional view of the medicament injector of FIG. 37, with the safety mechanism deployed in a primed position for use of the medicament injector;

FIG. 44 is a front perspective view of another example of a medicament injector, illustrating a mixed cartridge version;

FIG. 45 is left side view of the medicament injector of FIG. 44;

FIG. 46 is a front view of the medicament injector of FIG. 44;

FIG. 47 is a bottom view of the medicament injector of FIG. 44;

FIG. 48 is a longitudinal sectional view of the medicament injector of FIG. 44, with a safety mechanism of the medicament injector deployed in a safety position;

FIG. 49 is a longitudinal sectional view of the medicament injector of FIG. 44, with the safety mechanism deployed in a primed position for use of the medicament injector;

FIG. 50 is a bottom rear perspective view of the medicament injector of FIG. 44, with a cartridge compartment cover in an open position to expose a cartridge compartment in the injector housing and a saline cartridge and a medicament stock cartridge partially inserted in the cartridge compartment;

FIG. 51 is a bottom front perspective view of the medicament injector of FIG. 48, with the bottom housing subunit removed from the upper housing subunit of the injector housing;

FIG. 52 is a bottom front perspective view of the medicament injector of FIG. 49, with the bottom housing subunit removed from the upper housing subunit of the injector housing;

FIG. 53 is a front perspective view of an example of a disposable medicament injector;

FIG. 54 is an exploded front perspective view of the disposable medicament injector of FIG. 53, with the cartridge leveling disk detached from the main cartridge portion of the medicament cartridge;

FIG. 55 is a front view of the medicament injector of FIG. 53;

FIG. 56 is a left side view of the medicament injector of FIG. 53;

FIG. 57 is a longitudinal sectional view of the medicament injector of FIG. 53, with a safety mechanism of the medicament injector deployed in a safety position;

FIG. 58 is a longitudinal sectional view of the medicament injector of FIG. 53, with the safety mechanism deployed in a primed position for use of the medicament injector;

FIG. 59 is a bottom front perspective view of the medicament injector of FIG. 53, with the bottom housing subunit removed from the upper housing subunit of the injector housing;

FIG. 60 is a bottom front perspective view of the medicament injector of FIG. 53, with the bottom housing subunit removed from the upper housing subunit of the injector housing;

FIG. 61 is a perspective view of an example of a medicament dosing and loading device for loading medicament into a medicament cartridge of a medicament injector, with the device cover deployed in an open position on the device base of the device;

FIG. 62 is a side view of the medicament dosing and loading device of FIG. 61;

FIG. 63 is a perspective view of the medicament dosing and loading device of FIG. 61, with the device cover deployed in a closed position on the device base of the device;

FIG. 64 is a perspective view of the medicament dosing and loading device of FIG. 61, with the device cover open, and more particularly illustrating an example of insertion of a medicament injector fitted with a medicament cartridge into the device preparatory to loading and dosing of medicament into the medicament cartridge of the medicament injector;

FIG. 65 is a perspective view of the medicament dosing and loading device and the medicament injector inserted therein, as illustrated in FIG. 64, with the medicament injector inserted into the device;

FIG. 66 is a perspective view of the medicament dosing and loading device and the medicament injector inserted therein, as illustrated in FIG. 65, with the device cover closed on the device base;

FIG. 67 is a top view of the medicament dosing and loading device and the medicament injector inserted therein, as the medicament is loaded into the medicament cartridge of the medicament injector by operation of the device;

FIG. 68 is a top view of the medicament dosing and loading device and the medicament injector inserted therein, as illustrated in FIG. 67, upon completion of loading the medicament into the medicament cartridge;

FIG. 69 is a flow diagram of an example medicament injection method;

FIG. 70 is a flow diagram of another example medicament injection method; and

FIG. 71 is a flow diagram of another example medicament injection method.

FIG. 72 is another example of a custom medicament injection system;

FIG. 73 is an example of pre-treatment data about a treatment area associated with a person's skin;

FIG. 74 is an example of a relationship between an indica from the pre-treatment data of FIG. 73 and an animated muscle associated with the indicia;

FIG. 75 is a schematic example of a database relating different types of indicia to different target medicament injection site patterns;

FIG. 76 is a schematic example of a portion of a database relating different target medicament injection site patterns to a particular indicia;

FIG. 77 is an example of outputted targeting data in the form of an image displayed on a display of a computing device;

FIG. 78 is another example of pre-treatment data about a treatment area associated with a person's skin;

FIG. 79 is an example of a relationship between an indica from the pre-treatment data of FIG. 78 and an animated muscle associated with the indicia;

FIG. 80 is an example of outputted targeting data based on the pre-treatment data of FIG. 78;

FIG. 81 is another example of pre-treatment data about a treatment area associated with a person's skin;

FIG. 82 is an example of a relationship between an indica from the pre-treatment data of FIG. 81 and an animated muscle associated with the indicia;

FIG. 83 is an example of outputted targeting data based on the pre-treatment data of FIG. 81;

FIG. 84 is an example method of administering a botulinum toxin treatment; and

FIG. 85 is another example of a medicament injection device that uses a removably installed medicament cartridge and a removably installed liquid supply cartridge.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply examples of the inventive concepts defined in the appended claims.

All methods set forth in the present disclosure may be performed in any suitable order of steps unless otherwise indicated herein or contradicted by the rules of logic. The use of any and all examples or exemplary language provided herein is intended to clearly describe the subject matter of the disclosure and is not intended to be limiting on the scope of the subject matter set forth in the claims. No element, step, ingredient, or limitation mentioned or described in the specification shall not be construed as regarding any unclaimed component, step, or limitation to be essential in practicing the claimed subject matter.

Unless expressly or implicitly indicated otherwise, throughout the description and the appended claims, the terms “comprise”, “comprising”, “comprised of” and the like are to be construed in an inclusive sense as opposed to an exclusive or exhaustive sense, and are equivalent to the phrase, “including but not limited to”. Each embodiment disclosed herein can comprise, consist essentially of, or consist of its particular stated element, step, ingredient, or limitation. As used herein, the transition term “comprise”, “comprises”, “includes”, “including”, “has”, or “having” means “includes, but is not limited to, and allows for the inclusion of unspecified elements, steps, ingredients, or limitations, even in major amounts”. The transitional phrase “consisting of” excludes any element, step, ingredient, or limitation not specified. The transition phrase “consisting essentially of” shall limit the scope of the embodiment to the specified elements, steps, ingredients, or limitations and to those that do not materially affect the embodiment.

Unless otherwise noted using precise or limiting terminology, all numbers which express quantities of ingredients throughout the specification and claims are to be understood as being approximations of the numerical value cited to express the quantities of those ingredients. As used throughout the specification and claims, the term “about” has the meaning reasonably ascribed to it by a person skilled in the art when used in conjunction with a stated numerical value or range, i.e., denoting from the exact stated value or range to somewhat more or somewhat less than the stated value or range, from a deviation of from 0% with respect to the stated value or range to up to and including 20% of the stated value or range in either direction.

Various illustrative embodiments of the disclosure are described herein. Variations on the described illustrative embodiments may become apparent to those of ordinary skill in the art in reading the specification, drawings, and claims of the disclosure. Accordingly, the disclosure encompassed by the specification, claims and drawings includes all modifications Additionally, any combination of the elements in all possible variations thereof is encompassed by the subject matter of the disclosure unless otherwise indicated herein.

Illustrative embodiments of the disclosure relate to custom medicament injection systems, methods, and injectors which may be suitable for user self-injection of medicaments which may include but are not limited to botulinum toxin into or beneath the skin at one or more treatment areas on the person's skin. As used herein, “treatment area” may include but is not limited to the face, neck, arms, hands, thorax, abdomen, back, buttocks, legs, and feet. Various descriptions of the systems, methods, and injectors set forth herein refer to and describe the face of the patient as a treatment area for botulinum toxin injections. Hence, some examples in this description refer to a “facial scanner”, a “facial image” and a “facial injection map”, it will be recognized and understood that the following description and scope of the appended claims herein contemplates systems, methods, and injectors which are equally adaptable to other treatment areas on the patient. Hence, use of the terms, “face”, “facial”, face/area, facial injection/area, and the like shall be construed as including but not being limited to the face of the patient as a possible treatment area which may or may not be applicable, and additionally contemplates any other treatment area or areas, unless otherwise indicated.

As used herein, the terms “treat”, “treated”, “treatment”, and like terms may refer to therapeutic, cosmetic, and/or prophylactic treatment. In some applications of the medicament injection systems, methods, and injectors, subjects who are recipients of treatment may include human or animal subjects who have or have been diagnosed with a pathological and/or cosmetic disease or condition. In such cases, treatment may refer to administering to a subject in need of treatment a therapeutically effective amount of medicament such that the subject undergoes an improvement in at least one sign and/or symptom of the pathological and/or cosmetic disease or condition. The improvement may include any observable and/or measurable improvement. In some cases, treatment of the subject may merely improve one or more signs and/or symptoms of the disease or condition or may completely cure the subject of the disease or condition.

In some applications of the medicament injection systems, methods, and injectors, a “therapeutically effective amount” or an “effective amount” of medicament may be administered to a subject. As used herein, “therapeutically effective amount” or “effective amount” is an amount of medicament which is sufficient to decrease, suppress, or ameliorate at least one sign and/or symptom of the disease or condition. For example and without limitation, in various embodiments and applications, a quantity, volume, and/or number of injection dosage units of a medicament may range from less than about 0.1 mg to about 0.1 mg, from about 0.1 mg to about 100 mg, from about 0.5 mg to about 50 mg, from about 1.0 mg to about 25 mg, or from about 1.0 mg to about 10 mg, or from about 10 mg to more mg. In some embodiments, the number of injection dosage units of the medicament which is administered to the subject in each injection procedure may range from a fraction of a unit to about 1 unit, from about 1 unit to about 20 units, from about 20 units to about 40 units, from about 40 units to about 100 units, from about 100 units to about 200 units, from about 200 units to about 400 units, or from about 400 units to more units.

As used herein, the term “medicament” may include one or more medical, therapeutic, and/or bioactive compounds and/or formulations which may be used to treat the disease or condition. For example and without limitation, in some embodiments, the one or more medical, therapeutic, and/or bioactive compounds and/or formulations may include but are not limited to hyaluronic acid, botulinum toxin, platelet-rich plasma, polylactic acid, cannabinoids, vitamins, minerals, bimatoprost, minoxidil and/or other drugs or solutions. In some embodiments, the botulinum toxin may be derived from the Clostridium botulinum species.

In some embodiments, the medicament may include one or more medical, therapeutic, and/or bioactive compounds and/or formulations administered separately, together, or in combination with one or more therapies to treat the disease or condition.

In some embodiments, the medicament may include one or more neurotoxins selected from FDA approved neurotoxin products including but not limited to abobotulinumtoxinA, incobotulinumtoxinA, onabotulinumtoxinA, rimabotulinumtoxinB, prabotulinumtoxinA, daxibotulinumtoxinA, letibotulinumtoxinA, derivatives thereof, and mixtures thereof. The neurotoxins in the medicament may be natural, synthetic, or mixtures of natural and synthetic neurotoxins.

In some applications of the medicament injection systems, methods, and injectors, the medicament may be injected into muscle tissue at any of various anatomical sites on the body of the patient. These muscles may include but are not limited to the corrugator muscles, procerus muscles, lateral orbicularis muscles, frontalis muscles, detrusor, temporalis, occipitalis, cervical paraspinal, trapezius, biceps brachii, flexor carpi ulnaris, flexor carpi radialis, flexor digitorum sublimis (flexor digitorum superficialis), adductor pollices, flexor digitorum profundus, flexor pollicis longus, medical head of gastrocnemius, lateral head of gastrocnemius, soleus, tibialis posterior, flexor digitorum longus and flexor hallucis longus, brachialis, brachioradialis, flexor carpi ulnaris, medial and lateral pretarsal orbicularis of upper lid, lateral pretarsal orbicularis oculi of lower lid, and intradermally to axilla.

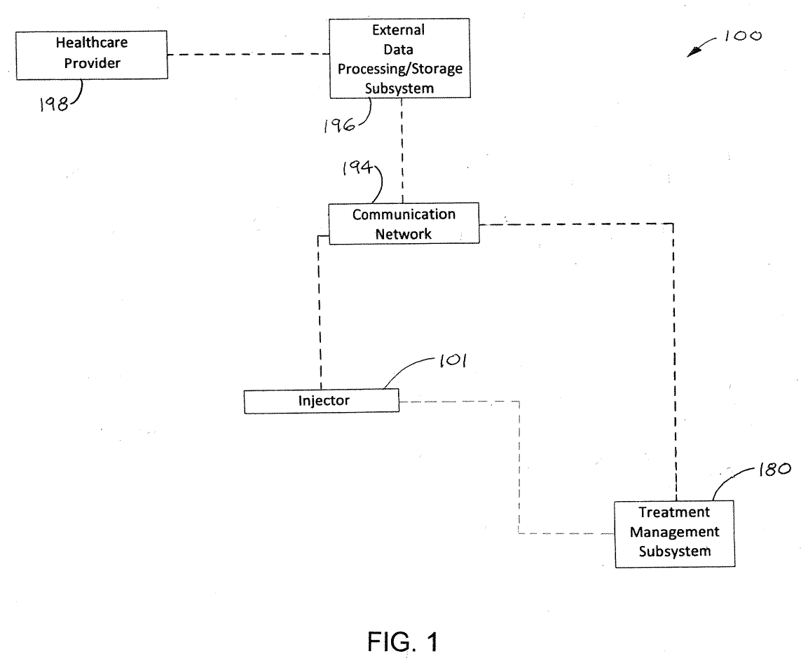

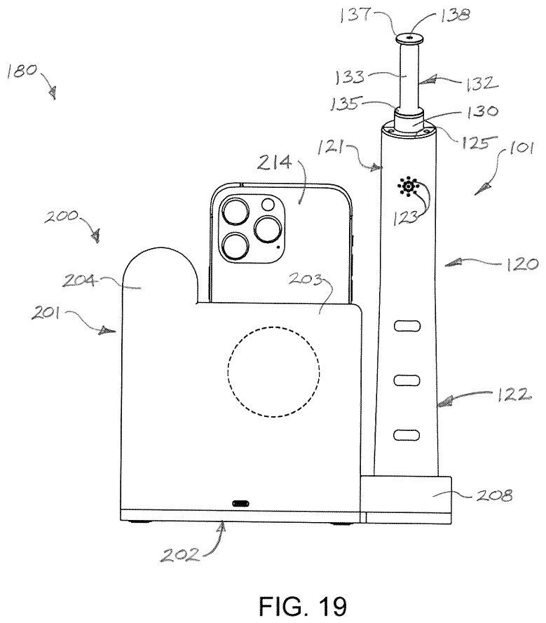

### FIGS. 1-22—Custom Medicament Injection System

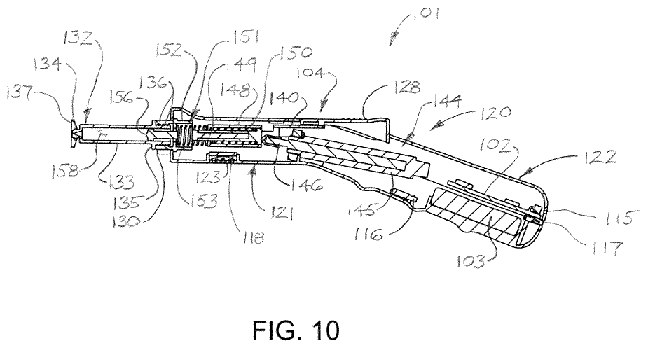

Referring initially to FIGS. 1-22 of the drawings, an illustrative embodiment of the medicament injection systems, hereinafter system 100, is illustrated in FIG. 1. The system 100 may include a medicament injector 101. The medicament injector 101 may be configured to facilitate epidermal, dermal, subdermal, subcutaneous, or intramuscular injection of a medicament 158 (FIG. 10) into or beneath the skin 220 on the face 219 on the body of a patient 218. For example and without limitation, in various embodiments, the medicament injector 101 may be configured to facilitate self-injection of the medicament 158 by the patient 218 or injection of the medicament 158 by an attending healthcare provider or untrained assisting person.

A custom treatment management subsystem 180 may communicably interface with the medicament injector 101 through a suitable communication network 194. Additionally or alternatively, the treatment management subsystem 180 may communicable interface directly with the medicament injector 101. In still other alternatives, the treatment management subsystem 180 need to communicate with the medicament injector 101, directly or indirectly. The treatment management subsystem 180 may be configured to guide and manage the injection procedures in each treatment session, particularly in cases of patient self-injection or injection by an untrained assisting person.

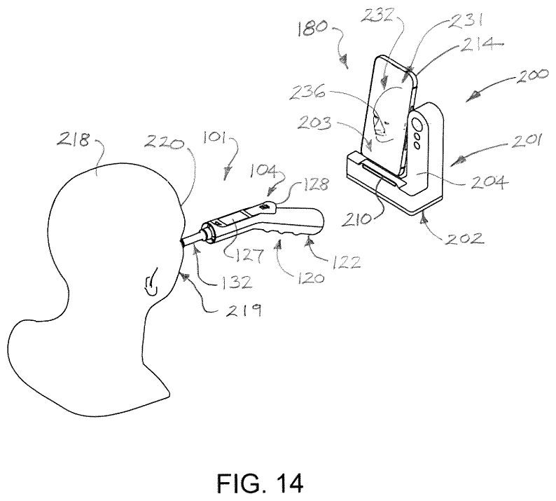

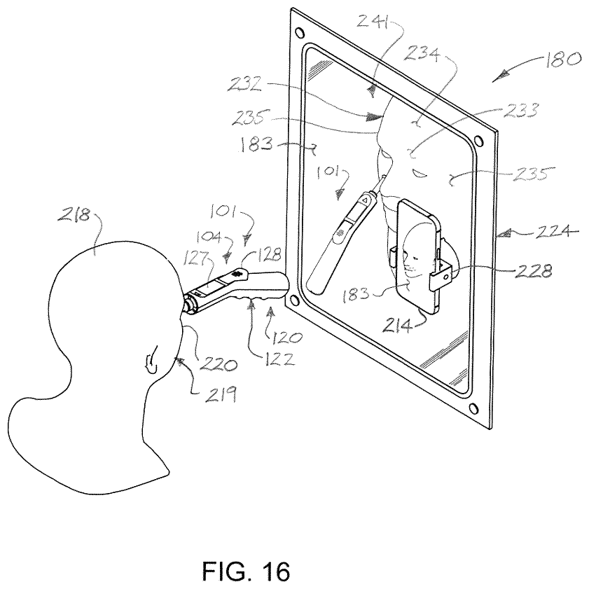

As illustrated in FIGS. 14, 15 and 17, in some embodiments, the treatment management subsystem 180 may include at least one personal electronic device 214. The personal electronic device 214 may include at least one smartphone or tablet computer, for example and without limitation. In some embodiments, the treatment management subsystem 180 may include at least one smart stand 200, which will be hereinafter described. The smart stand 200 may be configured to be used alone or in conjunction with the personal electronic device 214. As illustrated in FIG. 16, in some embodiments, the treatment management subsystem 180 may include at least one smart mirror 224. In various other embodiments, the treatment management subsystem 180 may include at least one desktop computer, laptop computer, and/or any other device, subsystem, web-based system or platform, or combination of devices or subsystems which, with supporting software, is configured and programmed to carry out the functions of the treatment management subsystem 180 described herein. In embodiments in which the treatment management subsystem 180 includes the personal electronic device 214, the software program for the various functions of the treatment management subsystem 180 may include an app which can be downloaded onto the device and installed. The app or other software may be configured to maintain statistics on injection procedures, treatment sessions, and other actions provided by the application after analyzing the obtained data. In some embodiments, the app may be configured to provide the capability for online purchase and shipping of medicament cartridges 132.

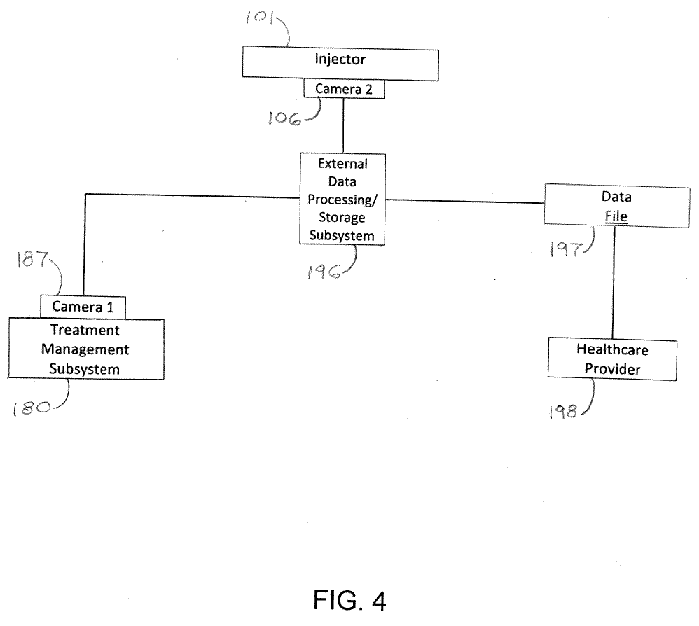

As illustrated in FIG. 1, at least one external data processing/storage subsystem 196 may communicably interface with the injector 101 and with the treatment management subsystem 180 via the communication network 194. The external data processing/storage subsystem 196 may be configured to store data files 197 (FIG. 4) which pertain to the injection procedures in each treatment session. The data files may include video files, audio files, audiovisual files, and/or text files, for example and without limitation. The external data processing/storage system 196 may include at least one server, cloud server, computer, laptop computer, personal electronic device, and/or any other device, subsystem, web-based system or platform, or combination of devices or subsystems which, with supporting software, is configured and programmed to carry out the functions of the external data processing/storage subsystem 196 described herein.

At least one remote healthcare provider 198 may communicably interface with the external data processing/storage system 196. The remote healthcare provider 198 may include at least one physician, clinic, physician's assistant, nurse practitioner, clinician, and/or other medical or healthcare personnel having knowledge or expertise in the injection procedures administered using the medicament injector 101. The external data processing/storage system 196 may provide the capability to render the data files 197 stored therein and/or transmitted therethrough by the patient 218 accessible for retrieval and viewing by the remote healthcare provider 198. The external data processing/storage system 196 may be configured to store and/or transfer data files 197 placed therein by the remote healthcare provider 198 and render the data files 197 accessible for retrieval and viewing by the patient 218 for instruction, guidance, advice, or feedback in carrying out current and/or subsequent injection procedures.

In some embodiments, the external data processing/storage subsystem 196 may form a component part of the injector 101 and/or the treatment management subsystem 180. Accordingly, the external data processing/storage subsystem 196 may be configured to communicate with the remote healthcare provider 198 either directly or via the communication network 194.

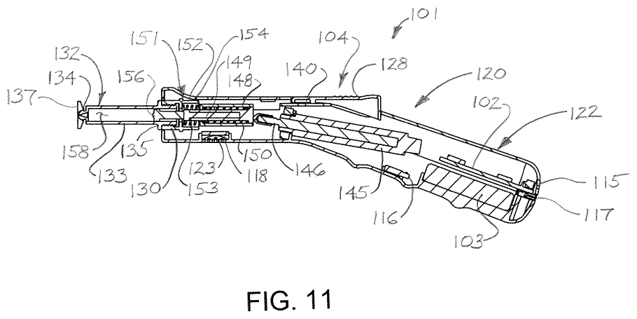

As illustrated in FIGS. 10 and 11 and will be hereinafter further described, the injector 101 may be configured to contain a removable medicament cartridge 132 which contains a supply of the medicament 158. In some embodiments, the medicament 158 may include botulinum toxin which may be injected into or beneath the skin 220 on the face 219 on the body of a patient 218 (FIG. 14) for treatment of wrinkles. In various embodiments, the medicament 158 may include any type of medical, therapeutic, or bioactive substance which is injectable into or beneath the skin 220 of the patient 218 for cosmetic, prophylactic, and/or therapeutic purposes. For example and without limitation, in some embodiments, the medicament 158 may include a therapeutic substance which may be used to treat undesirable muscle animation, hyperhidrosis, headaches, subcision, neocollagenesis, dermal thickening, depressed scarring, or hypertrophic scarring, for example and without limitation. Other therapeutic uses of the medicament 158 may include regeneration/remodeling of the dermis for cellulite and low dose “total facial carpeting” for reduction of pore size and improvement in the overall tone of the skin 220. In some embodiments, the medicament 158 may include an anesthetic. The quantity of medicament 158 which the medicament cartridge 132 contains may correspond to a single dose or multiple doses which is/are to be injected into or beneath the skin 220 of the patient 218. The injector 101 may be a needleless injector which is configured to generate and utilize a high-pressure jet of the medicament 158 to facilitate epidermal, dermal, subdermal, subcutaneous, or intramuscular injection of the medicament 158 into or beneath the patient's skin 220, as will be hereinafter described.

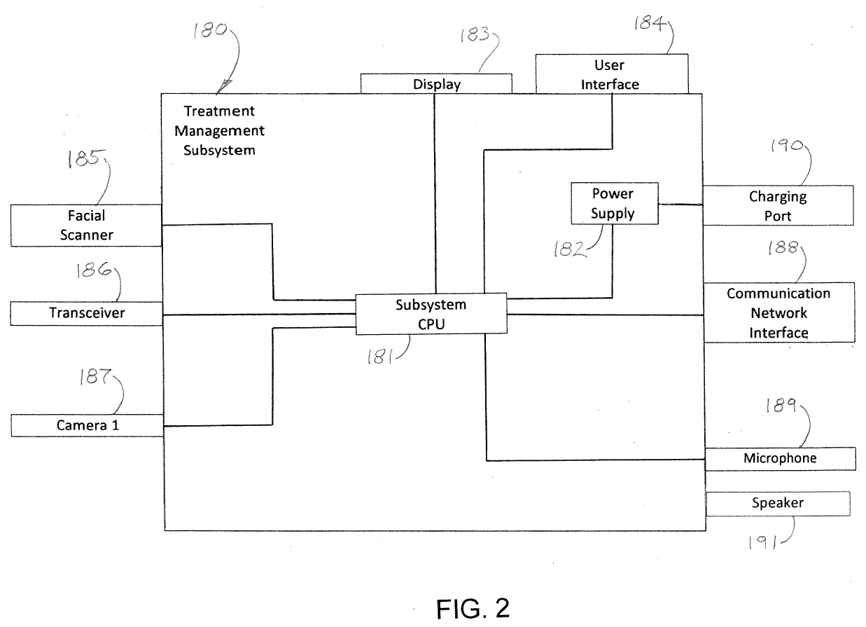

Various functional components which the treatment management subsystem 180 may include are illustrated in FIG. 2. Each of the functional components of the treatment management subsystem 180 described herein may include one or more hardware components, one or more software components, or combinations of one or more hardware components and one or more software components. The treatment management subsystem 180 may include a subsystem CPU 181. The subsystem CPU 181 may be configured to receive and process inputs and commands as well as implement the various commands and functions of the treatment management system 180. The subsystem CPU 181 may include data storage capabilities for the storage, retrieval, and transmittal of data files 197 (FIG. 4) which relate to the injection procedures in each treatment session. The subsystem CPU 181 may include at least one processor, controller, or combination of processors or controllers having processing and data storage capabilities and supporting software configured and programmed to carry out the functions of the treatment management subsystem 180 described herein.

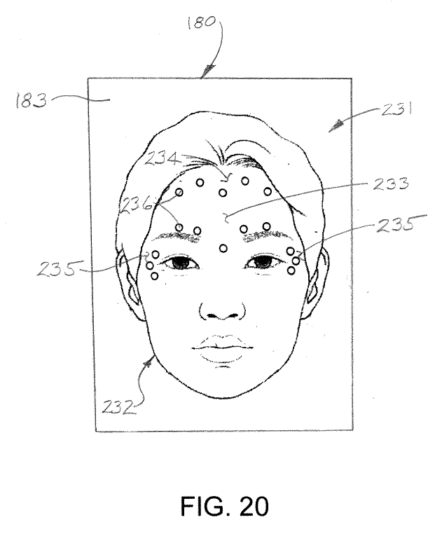

As illustrated in FIG. 2, at least one display 183 may interface with the subsystem CPU 181 of the treatment management subsystem 180. The display 180 may be configured to display the various functions and the status of each function of the treatment management subsystem 180. A facial scanner 185 may interface with the subsystem CPU 181. As illustrated in FIGS. 14 and 20 and will be hereinafter further described, in some embodiments, the facial scanner 185 may be configured to capture a 3-dimensional facial image 232 of the face 219 of the patient 218. In some embodiments, the facial scanner 185 may include a LIDAR (Light Detection And Ranging) scanner, for example and without limitation. In some embodiments, the facial scanner 185 may be a camera for capturing 2-dimensional images. In some embodiments, the facial scanner 185 may be a stand-alone device which may interface with the subsystem CPU 181 through a wireless or wired connection.

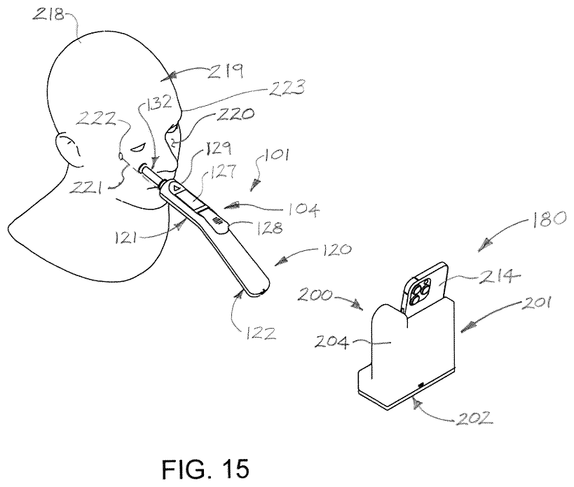

The subsystem CPU 181 may be configured to use the 3-dimensional or 2-dimensional image 232 captured by the facial scanner 185 to generate and present a virtual facial injection map 231 of the patient 218 on the display 183, as illustrated in FIG. 20. The facial injection map 231 may include the facial image 232 which represents the patient's face 219 and one or more virtual injection targets 236 superimposed on the facial image 232. The virtual injection targets 236 on the facial image 232 may serve as a guide for injection of the medicament 158 into or beneath the skin 220 of the patient 218 at the respective locations, positions, or physical injection sites or targets 222 (FIG. 15) which are on the skin 220 on the face 219 on the body of the patient 218 and correspond in position to the respective virtual injection targets 236. For example and without limitation, in some embodiments, the virtual injection targets 236 may serve as a guide for injection of botulinum toxin into or beneath the skin 220 of the patient 218 at injection sites marked by the respective physical injection targets 222 on the skin 220 corresponding to the positions of the respective virtual injection targets 217. As illustrated in FIG. 16, in some embodiments, the treatment management subsystem 180 may be configured to generate a reflected facial injection map 241 having a facial image 232 reflected from the patient 218 and the virtual injection targets 236 superimposed on the reflected facial image 232.

As further illustrated in FIG. 2, at least one power supply 182 may electrically interface with the subsystem CPU 181. In some embodiments, the power supply 182 may include at least one battery which may be disposable or rechargeable. In the latter case, a charging port 190 may interface with the power supply 182. The charging port 190 may be configured for connection to an external power source (not illustrated) such as a computer, device, or standard 120-volt household electrical outlet, for example and without limitation.

At least user interface 184 may interface with the subsystem CPU 181. The user interface 184 may enable the patient 218, the remote healthcare provider 198, or untrained assisting person to input various commands to control or program the subsystem CPU 181 in execution of the various functions of the treatment management subsystem 180. The user interface 184 may include at least one touchscreen display, buttons, knobs, switches, and/or other control features which may be suitable for the purpose.

In some embodiments, the user interface 184 of the treatment management subsystem 180 may be configured to normally maintain the functions of the treatment management subsystem 180 in a locked configuration and to unlock the functions of the treatment management subsystem 180 responsive to authorized user input. The authorized user input may be made via a fingerprint scanner (not illustrated), facial recognition via the facial scanner 185, and/or responsive to input of a password or code into the user interface 184, for example and without limitation.

At least one wireless transceiver 186 may operationally interface with the subsystem CPU 181. The transceiver 186 may be configured to wirelessly communicate with the medicament injector 101 for sharing of still and/or video images between the medicament injector 101 and the treatment management subsystem 180. In some embodiments, the transceiver 186 may be Bluetooth enabled.

As further illustrated in FIG. 2, at least one communication network interface 188 may interface with the subsystem CPU 181. The communication network interface 188 may be configured to interface with the communication network 194 (FIG. 1) for sharing of data files 197 between the treatment management system 180 and the external data processing/storage subsystem 196 or the remote healthcare provider 198, and/or between the treatment management system 180 and the medicament injector 101.

In some embodiments, at least one subsystem camera 187 may interface with the subsystem CPU 181 of the treatment management subsystem 180. The subsystem camera 187 may be configured to capture general still and/or video images of the face 219 of the patient 218 before, during and/or after each treatment session. The subsystem CPU 181 may be configured to store the captured images as data files 197 and may be configured to transmit the data files 197 to the remote healthcare provider 198, on command and/or in real time, via the communication network 188, the communication network 194 and the external data processing/storage system 196.

As further illustrated in FIG. 2, in some embodiments, a microphone 189 may interface with the subsystem CPU 181. The microphone 189 may be configured to capture verbal commands, comments, questions, and the like from the patient 218. The subsystem CPU 181 may be configured to store the verbal data as data files 197. The subsystem CPU 181 may be configured to transmit the verbal data files 197 to the remote healthcare provider 198 via the communication network interface 188, the communication network 194 and the external data processing/storage subsystem 196. In some embodiments, the subsystem CPU 181 may be configured to facilitate real-time communication of the patient 218 with the remote healthcare provider 198 via the microphone 189.

A speaker 191 may operationally interface with the subsystem CPU 181. The subsystem CPU 181 may be configured to access the verbal data files 197 received from the remote healthcare provider 198 and broadcast the voice of the remote healthcare provider 198 as the remote healthcare provider 198 provides instruction, guidance, feedback, or advice to the patient 218 on the injection procedures before, during, and/or after the treatment session. The subsystem CPU 181 may be configured to simultaneously present the video image of the remote healthcare provider 198 on the display 183.

In some embodiments, the subsystem CPU 181 may include a timer function. The subsystem CPU 181 may be configured to automatically provide periodic reminders of injection procedures or treatment sessions for the patient 218. The reminders may be visual, audible, or both. For example and without limitation, in some embodiments, the subsystem CPU 181 may be configured to visually present the reminder on the display 183. The reminders may be made after a predetermined time period (such as 3 months, for example and without limitation). The user interface 184 may provide the capability of the patient 218 to select, input, set, and adjust the time period between reminders.

As illustrated in FIGS. 14, 17 and 18, in some embodiments, the treatment management subsystem 180 may include the smart stand 200. The smart stand 200 may include a smart stand housing 201 which may contain some or all of the functional components of the smart stand 200. The smart stand housing 201 may include a stand base 202. The stand base 202 may have a device holding portion 203. The device holding portion 203 may be configured to support a smartphone or other personal electronic device 214 for viewing by the patient 218 during the treatment session. In some embodiments, the device holding portion 203 may have the capability to recharge the personal electronic device 214 when the device 214 is docked therein, according to the knowledge of those skilled in the art. A control portion 204 may extend from the device holding portion 203. The control portion 204 may include various features such as the facial scanner 185, the subsystem camera 187 and/or the speaker 191, for example and without limitation. In some embodiments, an injector holder 208 may extend from the stand base 202. The injector holder 208 may be suitably sized and configured to hold or support the medicament injector 101 when not in use.

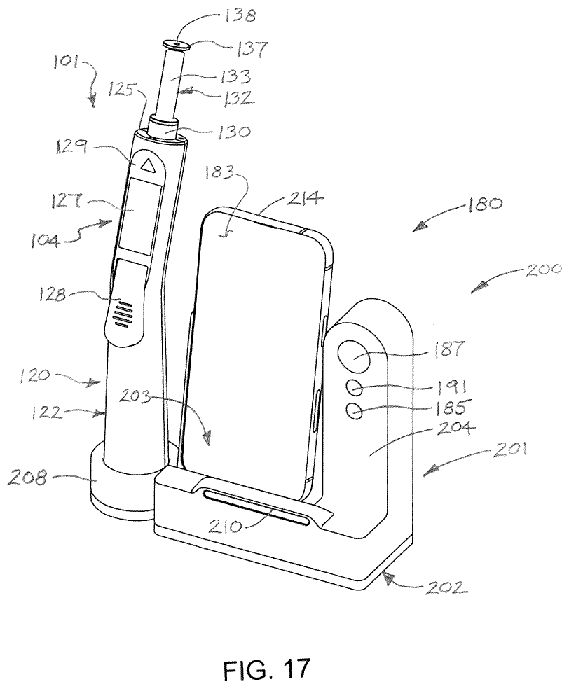

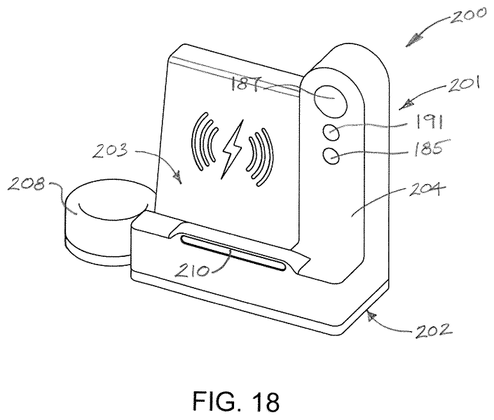

As illustrated in FIGS. 17 and 18, in some embodiments, at least one targeting LED 210 may be provided on the stand base 202 of the smart stand housing 201. The targeting LED 210 may have the same function or functions as the targeting LED 110 of the medicament injector 101 described further below.

As illustrated in FIG. 16, in some embodiments, the treatment management subsystem 180 may include the smart mirror 224. A device holding bracket 228 may be provided on the smart mirror 224. The device holding bracket 228 may be configured to support the personal electronic device 214. Accordingly, in some embodiments, the camera, facial scanning, communication, and other functions of the treatment management subsystem 180 may reside in the hardware and software configurations of the personal electronic device 214. The smart mirror 224 may be functionally linked to the personal electronic device 214 such that the personal electronic device 214 is configured to superimpose the virtual injection targets 236 (FIG. 20) on the facial image 232 of the reflected facial injection map 241 which is displayed on the smart mirror 224. In other embodiments, the camera, facial scanning, communication, and other functions of the treatment management subsystem 180 may be provided in or as component parts of the smart mirror 224.

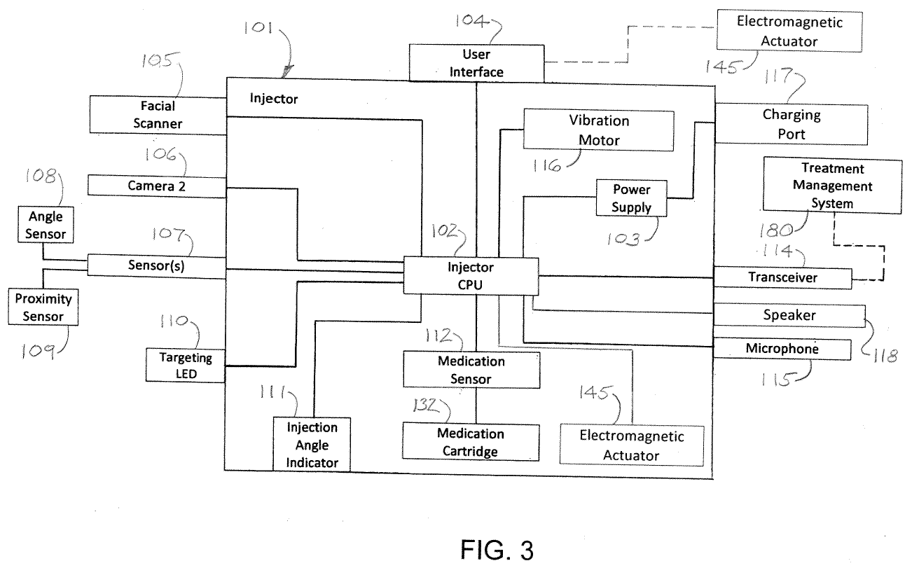

Various functional components which the medicament injector 101 may include are illustrated in FIG. 3. Each of the functional components of the medicament injector 101 described herein may include one or more hardware components, one or more software components, or combinations of one or more hardware components and software components. As illustrated in FIG. 3, the medicament injector 101 may include an injector CPU 102. The injector CPU 102 may include a printed circuit board (PCB) having the necessary processors, controllers, or combination of processors or controllers with the processing and data storage capabilities and supporting software configured to carry out the functions of the communication and other functions of the medicament injector 101 described herein.

At least one power supply 103 may electrically interface with the injector CPU 102. In some embodiments, the power supply 103 may include at least one battery which may be disposable or rechargeable. In the latter case, a charging port 117 may interface with the power supply 103. The charging port 117 may be configured for connection to an external power source (not illustrated) such as a computer, device, or standard 120-volt household electrical outlet, for example and without limitation.

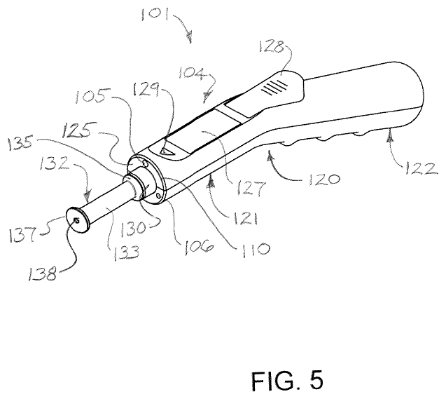

At least one user interface 104 may operationally interface with the injector CPU 102. The user interface 104 may enable the patient 218, the attending healthcare provider, or untrained assisting person to input commands in controlling or programming the injector CPU 102 to execute the various communication and other functions of the medicament injector 101. As illustrated in FIG. 5, in some embodiments, the user interface 104 may include at least one touchscreen display 127. The touchscreen display 127 may be configured to display such information as the power level for the power supply 103 and the quantity, volume, and/or number of injection dosage units of the medicament 158 which is in the medicament cartridge 132 on the medicament injector 101. The user interface 104 may include a trigger 128 which facilitates injection of the medicament 158 from the medicament cartridge 132 into or beneath the skin 220 of the patient 218, as will be hereinafter described. In various embodiments, the user interface 104 may additionally or alternatively include buttons, knobs, switches, and/or other control features suitable for the purpose. A power LED 129 (FIG. 5) may interface with the injector CPU 102 to indicate the on/off status of the medicament injector 101. The power LED 129 may be part of or adjacent to the user interface 104.

As illustrated in FIGS. 10-13, in some embodiments, the user interface 104 of the medicament injector 101 may include a fingerprint scanner 140. The injector CPU 102 may be configured to normally maintain the functions of the medicament injector 101 in a locked configuration and to unlock the functions of the medicament injector 101 responsive to authorized user input via the fingerprint scanner 140. Additionally or alternatively, the injector CPU 102 may be configured to unlock the functions of the medicament injector 101 responsive to input of facial recognition input via a facial scanner 105 (discussed below) or entry of a password or code into the user interface 104 via the touchscreen 127, for example and without limitation.

As further illustrated in FIG. 3, a facial scanner 105 may operationally interface with the injector CPU 102. In some embodiments, the facial scanner 105 may be of the same type and have the same functions as those which were heretofore described with respect to the facial scanner 185 (FIG. 2) of the treatment management system 180.

At least one injector camera 106 may interface with the injector CPU 102 of the medicament injector 101. The injector camera 106 may be configured to capture close-up still and/or video images of the face 219 on the patient 218 before, during, and/or after each treatment session. The injector CPU 102 may be configured to store the captured images as data files 197.

As illustrated in FIG. 4, in some embodiments, the camera feed from the subsystem camera 187 of the treatment management system 180 and the camera feed from the injector camera 106 of the medicament injector 101 may be combined to form a single video data file 197 which includes the close-up view of the patient's face 219 from the medicament injector 101 as well as the general view of the patient's face 219 or other area from the treatment management subsystem 180. The video data file 197 may be recorded to the external data processing/storage subsystem 196 and/or displayed in real-time to the remote healthcare provider 198. The subsystem camera 187 may provide a wide-angle overall view, whereas the injector camera 106 on the medicament injector 101 may provide the close-up view, of the patient's face 219.

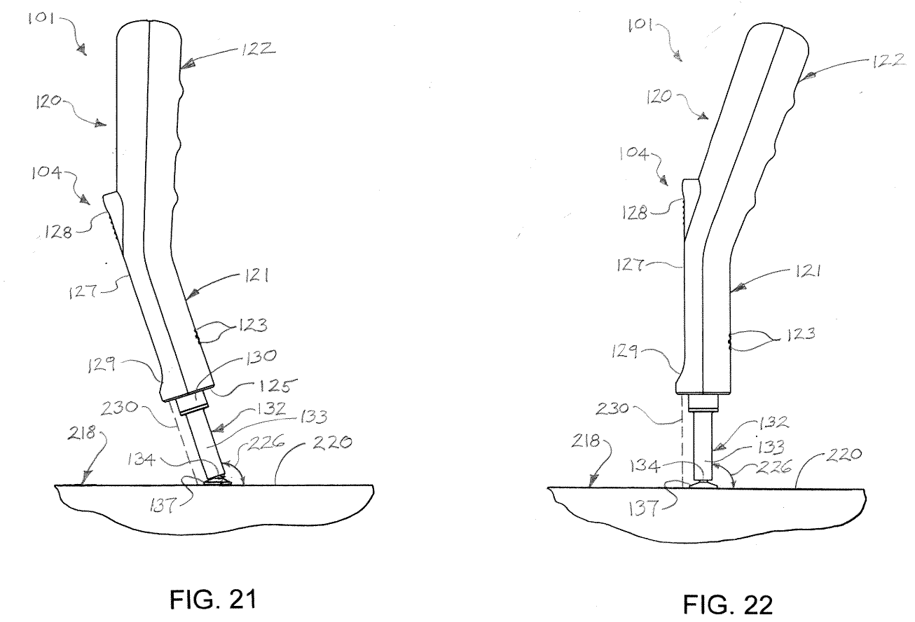

At least one sensor 107 may operationally interface with the injector CPU 102. In some embodiments, the sensor 107 may include at least one angle sensor 108. As illustrated in FIGS. 21 and 22 and will be hereinafter described, the angle sensor 108 may be configured to sense an injection angle 226 of the medicament injector 101 with respect to the skin 220 of the patient 218 preparatory to and/or during each injection procedure. The injector CPU 102 may be configured to verify whether the measured injection angle 226 corresponds to a predetermined correct or optimum injection angle 226 (e.g. perpendicular, or 90 degrees). As illustrated in FIG. 3, an injection angle indicator 111 may operationally interface with the injector CPU 102. The injection angle indicator 111 may include at least one visible indicator, at least one audible indicator, or both. Responsive to input from the injector CPU 102, the injection angle indicator 111 may be configured to indicate whether the angle sensor 108 senses the correct injection angle 226 prior to injection of the medicament 158. For example and without limitation, in some embodiments, the injection angle indicator 111 may include at least one light, such as a circular angle indicator light (not illustrated) which encircles an injector housing 120 of the medicament injector 101. The angle indicator light may illuminate a first color (such as red) when the injection angle 226 is not optimal and a second color (such as green) when the injection angle 226 is the optimal value. In some instances, the injector may be configured to prevent injection of the medicament unless the injector is at the proper angle or within a proper angle range relative to the person's skin.

In some embodiments, the sensor 107 may include at least one proximity sensor 109. The proximity sensor 109 may be configured to determine the distance between the medicament injector 101 and the skin 220 of the patient 218 preparatory to each injection procedure and to transmit the proximity data to the injector CPU 102. The injector CPU 102 may be configured to prevent injection of the medicament 158 into or beneath the skin 220 of the patient 218 if the proximity sensor 109 indicates that the medicament injector 101 is not in contact with the skin 220 for implementation of the injection procedure and to permit injection of the medicament 158 if the medicament injector 101 is in contact with the skin 220.

In some embodiments, at least one targeting LED 110 may operationally interface with the injector CPU 102. As illustrated in FIG. 15, the targeting LED 110 may be configured to emit a targeting LED beam 221 which illuminates at least one physical injection target 222 and/or the area on the skin 220 of the patient 218 which surrounds the physical injection target 222 as the LED beam 221 impinges on the skin 220. For example and without limitation, as illustrated in FIGS. 5-9, in some embodiments, the targeting LED 110 may be provided in the form of a ring which circumscribes the front housing surface 125 on the main housing portion 121 of the injector housing 120. In some embodiments, the physical injection target 222 on the skin 220 of the patient 218 may correspond to one of the virtual injection targets 236 (FIG. 20) which appears on the facial injection map 231 on the display 183 of the treatment management subsystem 180. The point of impingement of the light beam emitted by the targeting LED 110 onto the skin 220 may correspond to the injection site into which the medicament 158 is to be injected. Accordingly, alone or in conjunction with the virtual injection targets 236, the physical injection target 222 may serve as a guide for injection of the medicament 158 into or beneath the skin 220 of the patient 218 at the location or position on the face 219 on the body of the patient 218 which corresponds to the virtual injection target 236.

In at least some implementations of embodiments using targeting LED 110 or other functionality for providing visual or specific real-time feedback of target injection sites and the relative position and orientation of the injector relative to those target injection sites, the injector or other components of the system may include functionality for determining in real-time the relative position and orientation of the injector to those target injection sites. For example, in one implementation, the injector may include an inertial measurement unit (including components such as an accelerometer, gyroscope, and/or magnetometer) for determining and tracking changes in orientation which may be used in conjunction with a camera incorporated into the injector or another part of the system for determining and tracking changes in position and initially calibrating relative position and orientation of the injector to the target injection site(s). Other options for tracking position and orientation include Lighthouse tracking and similar techniques using lighthouse modules emitting signals that can be sensed and analyzed to determine relative position and orientation; Simultaneous localization and mapping (SLAM) and other technologies using cameras and machine learning algorithms; and Visual-inertial odometry (VIO) which uses sequential camera images to estimate positional and orientational changes. Other options may utilize fiducials mounted on the user and the injector for tracking their relative positions and orientation in six degrees of freedom using one or more cameras or other sensors (similar to tracking systems used in some types of Computer Assisted Surgery (CAS)). Many other technologies for tracking relative position and orientation of the injector and the target injection site(s) in six degrees of freedom are also known and may be readily incorporated into the injector and other parts of the custom medicament injection system.

With the target injection site(s) known (which may be determined in a variety of ways, as discussed in further detail in later parts of this document, and with the relative position and orientation of the injector to those target injection sites known, the custom medicament injection system may provide feedback to the user in a variety of ways to ensure that the injector is properly positioned and oriented relative to each injection site prior to injection. In just one example, this may take the form of the targeting LED beam 221 discussed above.

In still other implementations, it is not necessary to track the position and orientation of the injector relative to the target injection site(s) in order to provide feedback to the user for properly locating the injector relative to the injection sites. In some implementations, the image captured by the facial scanner, along with the determined target injection sties, may provide sufficient information for the system to be able to project a targeting LED beam onto the target injection site(s).

As further illustrated in FIG. 3, in some embodiments, at least one medication sensor 112 may operationally interface with the injector CPU 102. The medication sensor 112 may be configured to sense the quantity, volume, and/or number of injection dosage units of medicament 158 (FIG. 10) which is present in the medicament cartridge 132 after one or more injection procedures in each treatment session such as by using light reflection technology, for example and without limitation. The injector CPU 102 may be configured to indicate the quantity and/or volume of medicament 158, and/or determine the number of injection dosage units of the medicament 158, which are present in the medicament cartridge 132 on a readout, gauge, and/or other indicator which may be provided on the touchscreen display of the user interface 104, for example and without limitation.

In some embodiments, based on input from the medication sensor 112 and/or from another sensor 107 which is suitable for the purpose, the injector CPU 102 may have the capacity to determine whether each injection of the medicament 158 was a successful injection after the injection is completed. In some embodiments, a successful injection may constitute an injection in which a predetermined percentage of the intended quantity, volume, and/or number of injection dosage units of the medicament 158 was injected into or beneath the skin 220 of the patient 218 for the preceding injection. One factor which may affect the success of an injection is “spillback”, or the quantity of medicament 158 which does not inject into or beneath the skin 220 and is wasted during an injection. The injector CPU 102 may be configured to provide at least one notification indicating whether the injection was or was not successful audibly, visually, or both. For example and without limitation, in some embodiments, the injector CPU 102 may be configured to provide the notification on the touchscreen 127 of the user interface 104 and/or on the display 183 of the treatment management subsystem 180.

In some embodiments, at least one vibration motor 116 may interface with the injector CPU 102. The vibration motor 116 may be configured to impart vibration to the medicament injector 101 during the injection procedure to ensure smooth delivery of the medicament 158 from the medicament cartridge 132 into the patient 218.

A wireless transceiver 114 may operationally interface with the injector CPU 112. The wireless transceiver 114 may facilitate direct wireless communication with the treatment management system 180, as illustrated, and/or with the communication network 194 (FIG. 1). In some embodiments, the wireless transceiver 114 may be Bluetooth enabled.

As further illustrated in FIG. 3, in some embodiments, a microphone 115 may interface with the injector CPU 102. The microphone 115 may be configured to capture verbal commands, comments, questions, and the like from the patient 218. The injector CPU 102 may be configured to store the verbal data as data files 197. The injector CPU 102 may be configured to transmit the verbal data files 197 to the remote healthcare provider 198 either indirectly through the treatment management subsystem 180 or indirectly via the communication network 194 and the external data processing/storage subsystem 196, as illustrated in FIG. 1. In some embodiments, the injector CPU 102 may be configured to facilitate real-time communication of the patient 218 with the remote healthcare provider 198 via the microphone 115.

A speaker 118 may operationally interface with the injector CPU 102. The injector CPU 102 may be configured to access the verbal data files 197 received from the remote healthcare provider 198 and broadcast the voice of the remote healthcare provider 198 on command or in real time as the remote healthcare provider 198 provides instruction, guidance, feedback, or advice to the patient 218 on the injection procedures in the treatment session.

In some embodiments, the speaker 189 and the microphone 191 (FIG. 2) of the treatment management subsystem 180 may provide duplication of function with the speaker 118 and the microphone 115 (FIG. 3) of the medicament injector 101. Accordingly, any and all functions which are described herein with respect to the speaker 189 and the microphone 191 of the treatment management subsystem 180 may also apply to the speaker 118 and the microphone 115 of the medicament injector 101. Likewise, the facial scanner 185 of the treatment management subsystem 180 may provide duplication of function with the facial scanner 105 of the medicament injector 101. Accordingly, any and all functions which are described herein with respect to the facial scanner 185 of the treatment management subsystem 180 may also apply to the facial scanner 105 of the medicament injector 101. The injector CPU 102 of the medicament injector 101 may include the timer function which issues automatic, preset visual and/or audible reminders of treatment sessions, as was heretofore described with respect to the subsystem CPU 181 of the treatment management subsystem 180.

As illustrated in FIGS. 10 and 11 and will be hereinafter described, the medicament injector 101 may include a medicament injection assembly 144. The medicament injection assembly 144 may facilitate discharge and injection of the medicament 158 from the medicament cartridge 132 into or beneath the skin 220 of the patient 218. In some embodiments, the medicament injection assembly 144 may include an electromagnetic actuator 145. As illustrated in FIG. 3, in some embodiments, the electromagnetic actuator 145 of the medicament injection assembly 144 may interface with the user interface 104 through the injector CPU 102. Alternatively, in other embodiments, the electromagnetic actuator 145 may directly interface with the user interface 104 and may have its own dedicated control feature. For example and without limitation, as illustrated in FIGS. 10 and 11, in some embodiments, the user interface 104 may include the trigger 128 which facilitates operation of the electromagnetic actuator 145.

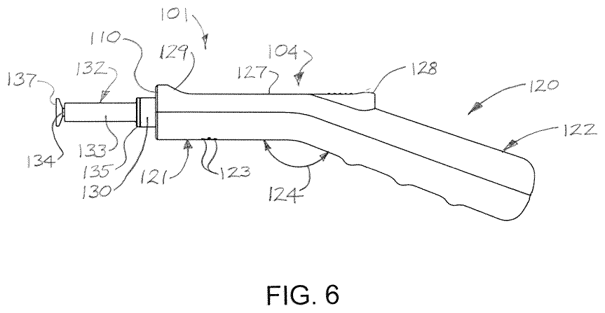

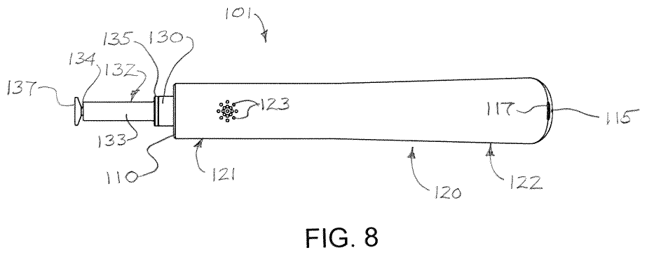

As illustrated in FIGS. 5-13, the medicament injector 101 may include an elongated injector housing 120. The injector housing 120 may have a main housing portion 121. A handle portion 122 may extend from the main housing portion 121. As illustrated in FIG. 6, in some embodiments, the longitudinal axis of the handle portion 122 may be disposed at an obtuse housing angle 124 with respect to the longitudinal axis of the main housing portion 121. The angled or curved orientation of the handle portion 122 with respect to the main housing portion 121 may enable the injector housing 120 to fit comfortably and ergonomically in the hand of the patient 218 while enabling the patient 218 to attain and maintain the correct or optimum injection angle 226 (FIG. 22) during each injection procedure. In some embodiments, the injector housing 120 may be fabricated in separable housing subunit (not illustrated) to facilitate access to the interior of the injector housing 120. The housing subunits may be detachably attachable or snap-fitted to each other via multiple tabs and interfacing tab openings (not illustrated) on the respective subunits. As illustrated in FIG. 8, at least one speaker opening 123 may be provided in the lower portion of the main housing portion 121 of the main housing 120. The speaker opening 123 may interface with the speaker 118 (FIG. 3).

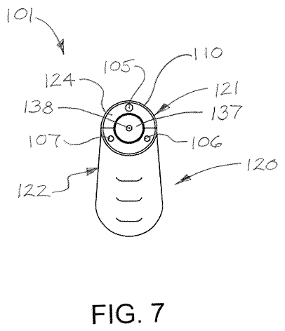

The main housing portion 121 of the main housing 120 may have a front housing surface 125. The front housing surface 125 may be disposed within a plane which is perpendicular to the longitudinal axis of the main housing portion 121. As illustrated in FIG. 7, in some embodiments, the facial scanner 105, the injector camera 106, and/or the sensor or sensors 107, which may include the angle sensor 108 and/or the proximity sensor 109, heretofore described with respect to FIG. 3, may be provided on the front housing surface 125. Additionally or alternatively, the injection angle indicator 111 (FIG. 3) may be provided on the front housing surface 125.

As illustrated in FIG. 5, the user interface 104 of the medicament injector 101 may be provided in any suitable accessible location or position on the exterior of the injector housing 120 which renders the user interface 104 easily manipulatable by the patient 218 during each self-injection procedure or by an attending healthcare provider or untrained assisting person during the procedure. Accordingly, in some embodiments, the touchscreen display 127 of the user interface 104 may be provided on the upper area of the main housing portion 121. The trigger 128 for the electromagnetic actuator 145 (FIG. 10) of the medicament injection assembly 144 may be provided behind the touchscreen display 127. The power LED 129 may be provided in front of the touchscreen display 127. However, in other embodiments, the location or position and arrangements of these components of the user interface 104 may be in alternative positions or locations on the injector housing 120.

The microphone 115 and the charging port 117 may be located at any suitable location or position on the injector housing 120. For example and without limitation, as illustrated in FIG. 8, in some embodiments, either or both of these components may be provided at the rear end area of the handle portion 122 of the injector housing 120.

Exemplary locations and positions of various internal components of the medicament injector 101 are illustrated in FIGS. 10-13. Accordingly, the injector CPU 102 and the power supply 103 may be provided in the handle portion 122 of the injector housing 120. The vibration motor 116 may be provided in the handle portion 122, as illustrated, or alternatively, in the main housing portion 121 in contact with the wall of the injector housing 120. The speaker 118 may be provided in the main handle portion 121 adjacent to the speaker openings 123. The electromagnetic actuator 145 of the medicament injection assembly 144 may be provided in the main housing portion 121 and the handle portion 122, as illustrated. In various alternative embodiments, these components may be provided in alternative locations or positions within the injector housing 120.

The medicament cartridge 132 may include a main cartridge portion 133 which may be generally elongated and cylindrical. In some embodiments. The main cartridge portion 133 may be plastic and transparent or translucent. The main cartridge portion 133 may contain at least the selected quantity, volume, and/or number of injection dosage units of the medicament 158 (FIGS. 10 and 11) which is/are to be injected during each injection procedure. In some embodiments, volume, quantity and/or injection dosage unit markings (not illustrated) for the medicament 158 may be provided on the main cartridge portion 133.

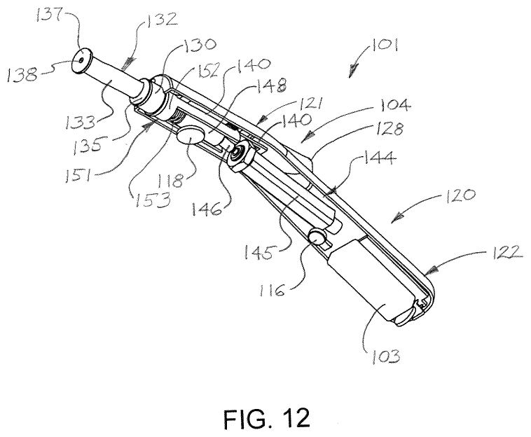

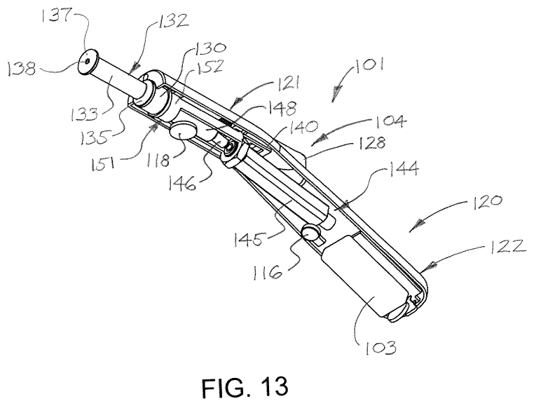

An injection nozzle 134 may be provided at the forward end of and in fluid communication with the main cartridge portion 133. In some embodiments, a cartridge leveling disk 137 may protrude outwardly from the injection nozzle 134. As illustrated in FIGS. 12 and 13, the cartridge leveling disk 137 may have a central injection opening 138 which fluidly communicates with the injection nozzle 134. In some embodiments, the injection nozzle 134 and the cartridge leveling disk 137 may be fabricated of rubber, plastic, and/or other resilient material.

As illustrated in FIGS. 21 and 22, in some embodiments, the cartridge leveling disk 137 may be pivotally or swivelly mounted on the main cartridge portion 133 to facilitate adjustments in the injection angle 226 after engagement of the cartridge leveling disk 137 against the skin 220 of the patient 218 preparatory to each injection procedure. In other embodiments, the cartridge leveling disk 137 may be mounted at a fixed angle or orientation with respect to the main cartridge portion 133 such that the plane of the cartridge leveling disk 137 is perpendicular to the longitudinal axis of the main cartridge portion 133. This expedient may ensure that the injection angle 226 of the medicament cartridge 132 remains optimal (e.g. 90 degrees or perpendicular) upon engagement of the cartridge leveling disk 137 with the patient's skin 220 and throughout the injection procedure.

As illustrated in FIGS. 10 and 11, a cartridge plunger 156 may be slidably disposed in the main cartridge portion 133 of the medicament cartridge 132. Accordingly, as it slides in the main cartridge portion 133 forwardly, or toward the injection nozzle 134, the cartridge plunger 156 may be configured to expel or discharge the medicament 158 from the main cartridge portion 133 through the injection nozzle 134 and the injection opening 138 in the cartridge leveling disk 137, respectively, in injection of the medicament 158 into or beneath the skin 220 of the patient 218 during each injection procedure in the treatment session.

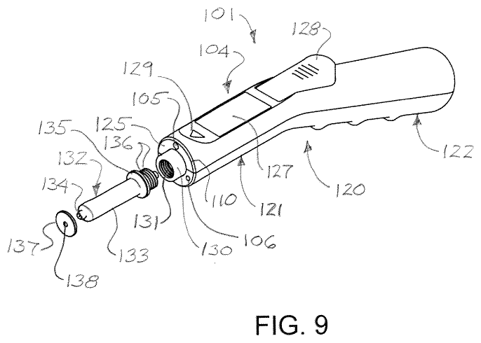

The main cartridge portion 133 of the medicament cartridge 132 may be configured for detachable attachment to the front end of the main housing portion 121 of the injector housing 120 using any technique which is suitable for the purpose. For example and without limitation, as illustrated in FIGS. 9-11, in some embodiments, an interiorly-threaded cartridge attachment nipple 130 may be provided on the main housing portion 121 of the injector housing 120. The cartridge attachment nipple 130 may protrude forwardly from the front housing surface 125 of the main housing portion 121. Exterior cartridge threads 136 may be provided at the rear end portion on the main cartridge portion 133. The exterior cartridge threads 136 may be configured to detachably engage the interiorly threaded cartridge attachment nipple 130 to facilitate removable attachment of the medicament cartridge 132 to the main housing portion 121 of the injector housing 120. A circumferential cartridge flange 135 may extend outwardly from the main cartridge portion 133 adjacent to the cartridge threads 136. The cartridge flange 135 may engage the protruding forward end of the cartridge attachment nipple 130 when the medicament cartridge 132 is fully threaded into the cartridge attachment nipple 130. In other embodiments, the main cartridge portion 133 may be detachably attachable to the main housing portion 121 using a friction fit, interference fit, clip, clamp, snap on, and/or other attachment technique known by those skilled in the art and suitable for the purpose.

As illustrated in FIGS. 10 and 11, the electromagnetic actuator 145 of the medicament injection assembly 144 may include an extendable and retractable actuator piston 146. The actuator piston 146 may be selectively deployable in a retracted actuator piston position (illustrated in FIGS. 10 and 11) and an extended actuator piston position (not illustrated) in the injector housing 120. In the retracted actuator piston position, the actuator piston 146 may be retracted rearwardly, or away from the medicament cartridge 132, as illustrated. In the extended actuator piston position, the actuator piston 146 may be extended forwardly, or toward the medicament cartridge 132 in the injector housing 120. The trigger 128 of the user interface 104 may operably interface with the electromagnetic actuator 145 in such a manner as to facilitate extension and retraction of the actuator piston 146. A single depression or actuation of the trigger 128 may facilitate deployment of the actuator piston 146 from the retracted actuator piston position to the extended actuator piston position. Subsequent release of the trigger 128 may facilitate retraction of the actuator piston 146 from the extended actuator piston position back to the retracted actuator piston position.

A plunger piston housing 148 may be provided in the main housing portion 121 of the injector housing 120, forwardly of the actuator piston 146. A plunger piston 149 may be slidably disposed between a retracted plunger piston position (illustrated in FIGS. 10-13) and an extended plunger piston position in the plunger piston housing 148. The electromagnetic actuator 145 may be suitably positioned in the interior of the injector housing 120 such that the actuator piston 146 engages the plunger piston 149, as illustrated. As it is deployed from the retracted actuator piston position toward the extended actuator piston position, the actuator piston 146 may apply forward pressure against the plunger piston 149 to displace the plunger piston 149 forwardly in the plunger piston housing 148 from the retracted plunger piston position to the extended plunger piston position, against the cartridge plunger 156 of the medicament cartridge 132. Continued extension of the actuator piston 146 to the extended actuator piston position may cause the plunger piston 149 to advance the cartridge plunger 156 forwardly in the main cartridge portion 133 of the medicament cartridge 132 such that the cartridge plunger 156 forcefully expels or discharges at least a predetermined quantity, volume, and/or number of injection dosage units of the medicament 158 from the main cartridge portion 133 through the injection nozzle 134 and the injection opening 138 in the cartridge leveling disk 137, respectively.

In some embodiments, the electromagnetic actuator 145 may be configured and calibrated such that the reach of the actuator piston 146 on full extension to the extended actuator piston position causes the plunger piston 149 to advance the cartridge plunger 156 to the distal or forward end of the main cartridge portion 133, thereby having discharged the entire quantity, volume, and/or number of injection dosage units of the medicament 158 from the main cartridge portion 133 through the injection nozzle 134 for each deployment of the actuator piston 146. Accordingly, the electromagnetic actuator 145 may be configured and calibrated such that the reach of the actuator piston 146 for each pull, depression, or actuation of the trigger 128 causes the plunger piston 149 to advance the cartridge plunger 156 along the entire length and volume of the main cartridge portion 133. In some embodiments, the electromagnetic actuator 145 may be configured and calibrated such that the reach of the actuator piston 146 for each pull, depression or actuation of the trigger 128 causes the plunger piston 149 to advance the cartridge plunger 156 along an increment or portion of the length and volume of the main cartridge portion 133 which corresponds to ejection or discharge of the predetermined quantity, volume, and/or number of injection dosage units of the medicament 158 for each injection procedure in the treatment session.

In some embodiments, a plunger piston return spring 150 may be provided in the plunger piston housing 148. The plunger piston return spring 150 may engage and normally bias the plunger piston 149 in the retracted plunger piston position in the plunger piston housing 148. Accordingly, as the actuator piston 146 of the electromagnetic actuator 145 deploys the plunger piston 149 from the retracted plunger piston position to the extended plunger piston position in the plunger piston housing 148, the plunger piston 149 may advance in the plunger piston housing 148 against the bias imparted by the plunger return spring 150. Upon subsequent return of the actuator piston 146 from the extended actuator piston position to the retracted actuator piston position, the plunger piston return spring 150 may return the plunger piston 149 from the extended plunger piston position back to the retracted plunger piston position in the plunger piston housing 148.

As further illustrated in FIGS. 10-13, in some embodiments, a safety mechanism 151 for the medicament cartridge 132 may be provided in the main housing portion 121 of the injector housing 120. Accordingly, the cartridge attachment nipple 130 may be slidably mounted within a nipple opening (not numbered) provided in the front housing surface 125 of the main housing portion 121. A safety spring housing 152 may extend from the cartridge attachment nipple 130 into the interior of the main housing portion 121. The cartridge attachment nipple 130 and the safety spring housing 152 may be selectively deployable in a safety position (illustrated in FIGS. 10 and 12) and a primed position (FIGS. 11 and 13) for use of the medicament injector 101. As illustrated in FIGS. 10 and 12, in the safety position, the cartridge attachment nipple 130 may protrude beyond the front housing surface 125 of the main housing portion 121, exterior to the main housing portion 121, with the safety spring housing 152 inside the main housing portion 121 such that the plunger piston 149 disengages the cartridge plunger 156 and a gap (not numbered) exists therebetween. In the primed position, illustrated in FIGS. 11 and 13, the cartridge attachment nipple 130 may be retracted into the main housing portion 121 such that the gap between the plunger piston 149 and the cartridge plunger 156 is closed and the plunger piston 149 engages the cartridge plunger 156.

As illustrated in FIGS. 10-12, the safety mechanism 151 may include a safety spring 153 provided in the safety spring housing 152 between and in engagement with the plunger piston housing 148 and the safety spring housing 152. The safety spring 153 may normally bias the cartridge attachment nipple 130 and the safety spring housing 152 in the safety position illustrated in FIG. 10, with the gap between the plunger piston 149 and the cartridge plunger 156. As the medicament cartridge 132 is deployed from the safety position to the primed position preparatory to the injection procedures, against the bias imparted by the safety spring 153, the gap between the plunger piston 149 and the cartridge plunger 156 may be closed until the plunger piston 149 engages the cartridge plunger 156, as illustrated in FIGS. 11 and 13. The safety spring housing 152 may engage the main housing portion 121 or may engage a catch 154 (FIG. 11) provided in the interior of the main housing portion 121 to maintain the medicament cartridge 132 in the primed position. In some embodiments, the medicament cartridge 132 may be deployed from the safety position to the primed position such as by application of manual pressure to the medicament cartridge 132 in the rearward direction, or towards the main housing portion 121. In other embodiments, a lever or other mechanism (not illustrated) may facilitate selective deployment of the medicament cartridge 132 from the safety position to the primed position.

In some embodiments, the plunger piston 149 may be omitted and the actuator piston 146 of the electromagnetic actuator 145 may be configured to directly engage the cartridge plunger 156 of the medicament cartridge 132. Accordingly, upon deployment from the retracted actuator piston position to the extended actuator piston position, the actuator piston 146 may deploy the cartridge plunger 156 in the main cartridge portion 133 of the medicament cartridge 132 for discharge of some or all of the medicament 158 from the medicament cartridge 132.

The electromagnetic actuator 145 of the medicament injection assembly 144 may include any type of actuator or combination of actuators which facilitate(s) deployment of the actuator piston 146 from the retracted actuator piston position to the extended actuator piston position using electromagnetic technology. For example and without limitation, in some embodiments, the electromagnetic actuator 145 may include a Lorentz-force actuator. Lorentz-force actuators are capable of providing needle-free drug delivery by jet injection by ejecting a liquid through a narrow orifice at high pressure, thereby creating a fine high-speed fluid jet that can readily penetrate skin and tissue. Lorentz-force actuators may facilitate continuous monitoring and modulation of the speed of a drug jet as well as precise regulation in the quantity, volume, and/or number of injection dosage units of injectant delivered during each injection procedure. Lorentz-force actuators have been shown to facilitate control of injection depths of up to 16 mm. Linear Lorentz-force actuators and other actuators in needle-free jet injection which may be suitable for adaptation as the electromagnetic actuator 145 are described in U.S. Pub. No. 2017/0143906, U.S. Pub. No. 2018/0147351, and U.S. Pat. No. 8,740,838.

In some applications, the medicament injector 101 may be used to inject botulinum toxin into one or more physical injection targets or sites 222 (FIG. 15) on the skin 220 on the face 219 on the body of a patient 218 for the cosmetic treatment of wrinkles. Accordingly, the medicament 158 in the medicament cartridge 132 may include botulinum toxin. In other applications, the medicament injector 101 may be used to inject other therapeutic and/or cosmetic substances as the medicament 158 into or beneath the skin 220 for other treatment regimens. For example and without limitation, the medicament injector 101 may be used to inject medicament 158 into or beneath the skin 220 to treat such conditions as tissue necrosis, depressed scars, facial atrophic acne scars, herpes zoster-induced depressed scars, hypertrophic scars, thyroidectomy scars, keloids, or wrinkles. Accordingly, the medicament 158 may include formulations of normal saline (for treatment of tissue necrosis, depressed scar, hypertrophic scar, thyroidectomy scar); hyaluronic acid diluted with hypertonic glucose (facial atrophic acne scar); or hyaluronic acid (facial atrophic acne scar, herpes zoster-induced scars, keloids, wrinkles). Various non-limiting examples of clinical applications suitable for the medicament injector 101 and treatment parameters and protocols therefor are illustrated in Table I below. The operational description of the medicament injection system 100 which is set forth herein with respect to the injection of botulinum toxin as the medicament 158 can be adapted to these and other clinical applications according to the knowledge of those skilled in the art.

TABLE I
Clinical application of medicament injectors in skin remodeling

| Skin condition | Parameters | Treatment protocol |
|---|---|---|
| Tissue necrosis following | Nozzle diameter: 100 μm | 1 treatment |
| filler injection | Velocity: 108 m/s | |
| | 0.15 ml of normal saline at 2 | |
| | mm distance | |
| Depressed scar in pediatric | Nozzle diameter: 100 μm | 8 treatments over 1-month |
| patient | Velocity: 108 m/s | interval |
| | 0.1 ml of normal saline at 2 | |
| | mm distance | |
| Facial atrophic acne scar | Nozzle diameter: 200 μm | 1 treatment |
| | 50% pressure power | |
| | 85 uL hyaluronic acid | |
| | diluted with 20% hypertonic | |
| | glucose | |
| Facial atrophic acne scar | Nozzle diameter: 150 μm | 3 treatments over 1-month |
| | 10*10 mm square-shaped | interval |
| | nozzle tip | |
| | 70% pressure power | |
| | 0.15 ml hyaluronic acid | |
| | 20-50 shots according to | |
| | patient scar condition | |
| Herpes zoster-induced | Nozzle diameter: 150 μm | 6 treatments over 1-month |
| depressed scars | 10*10 mm square-shaped | interval |
| | nozzle tip | |
| | 80% pressure power | |
| | 0.2 ml hyaluronic acid | |
| Hypertrophic scar on | Nozzle diameter: 100 μm | 2 treatments over 1-month |
| forehead | Velocity: 180 m/s | interval |
| | 0.1 ml normal saline at 2 | |
| | mm distance | |
| Thyroidectomy scar | Nozzle diameter: 100 μm | 4 treatments over 1-month |
| | Velocity: 108 m/s | interval |
| | 0.1 ml normal saline at 2- | |
| | mm distance | |
| Keloids | Nozzle diameter: 150 μm | 3 treatments over 3-week |
| | 10*10 mm square-shaped | interval |
| | nozzle tip | |
| | 70% pressure power | |
| | 0.15 ml hyaluronic acid | |
| Wrinkles on face, neck, | Nozzle diameter: 150 μm | 3-4 treatments over 3-4 |
| chest and dorsal hands | 10*10 mm square-shaped | week interval |
| | nozzle tip | |
| | 70% pressure power | |
| | Approximately 2 mg HA | |
| | deposited into each 5 cm² | |
| | treatment area | |

For botulinum injection in the treatment of wrinkles, the physical injection targets 222 into which the botulinum toxin medicament 158 is injected may be, without limitation, in the glabella, forehead, and/or temples on the patient's face 219. The botulinum toxin can be selected from the group consisting of botulinum toxin types A, B, C1, D, E, F and G, a pure or purified (i.e. about 150 kD) botulinum toxin, as well as a native or recombinant botulinum toxin, for example and without limitation. The material can comprise between about 1 unit to about 400 units of the botulinum toxin or a therapeutically effective amount, and the composition can comprise an amount of botulinum toxin sufficient to achieve a therapeutic effect lasting between 1 month and 5 years. The botulinum toxin can be reconstituted within the device as described elsewhere herein or before the medicament cartridge 132 is placed on the medicament injector 101. The botulinum toxin can be reconstituted with sterile 0.9% sodium chloride (saline).

The dilution ratio of the medicament 158 can be 1 to 100 units of botulinum toxin per 0.1 ml of saline, and more preferably, 1 to 50 units per 0.1 ml of saline, or 1 to 10 units per 0.1 ml of saline. In some embodiments, 4 units per 0.1 mL of saline can be used. The dilution ratio may be highly dependent on the type of botulinum toxin used or combination of botulinum toxins used.

In some embodiments, the medicament cartridge 132 may contain a single injection dosage unit of the botulinum toxin medicament 158. In other embodiments, the medicament cartridge 132 may contain multiple injection dosage units of the botulinum toxin medicament 158. Accordingly, the electromagnetic actuator 145 may be configured and calibrated to inject the quantity or volume of medicament 158 corresponding to one or more injection dosage units into each physical injection target 222 responsive to a single actuation or depression of the trigger 128 of the user interface 104. The quantity, volume or number of injection dosage units of the medicament 158 which is/are to be injected for each actuation of the trigger 128 may be programmable by user input via the user interface 104. Each injection dose corresponding to a particular volume, quantity or one or more injection dosage units of the medicament 158 may be injected into a corresponding injection site at a physical injection target 222 on an area (glabella, forehead, temple, etc.) on the face 219 of the patient 218.

For botulinum toxin injections, an example dosage quantity or volume in each injection dosage unit of the medicament 158 may fall within the range of from about 0.1 cc to about 0.3 cc. In other types of medicament injections, the quantity or volume of the medicament 158 in each injection dosage unit may vary. The quantity or volume and number of the injection dosage units for each injection dose, as well as the injection depth, may depend on the area of the face 219 at which the injection is made, as well as the number and extent of wrinkles in the area. The quantity, volume or number of injection dosage units of the medicament 158 may be tailored according to such factors as the depth of the wrinkles and the mass of the muscle which is targeted. In some applications, each area of the patient's face 219 may require multiple injection dosage units. Each medicament cartridge 132 may contain a sufficient number of injection dosage units of the medicament 158 to facilitate a single injection, the multiple injections required for a particular area on the face 219, or the injections required for all of the areas on the face 219. For example and without limitation, in some embodiments, the medicament cartridge 132 may contain one injection dosage unit, at least 5 injection dosage units or up to 100 injection units of the medicament 158 such as in 5-unit increments. The number of injection dosage units of the medicament 158 contained in each medicament cartridge 132 may be limited by the size and design constraints of the medicament cartridge 132 and the medicament injector 101.

The electromagnetic actuator 145 of the medicament injection assembly 144 may be configured to produce a high-velocity jet of the medicament 158 from the injection nozzle 134 of the medicament cartridge 132. For example and without limitation, in some embodiments, the electromagnetic actuator 145 may be configured and calibrated and the diameter of the injection nozzle 134 selected to eject each injection dose of the medicament 158 at a jet velocity of from about 100 m/s to about 200 m/s, and preferably, from about 110 m/s to about 190 m/s. For botulinum toxin injections, the jet velocity and force may depend on such factors as the area of the face 219 or other body area, the thickness and Young's modulus of the skin 220 and the muscle which is targeted. The nozzle diameter of the injection nozzle 134 of the medicament cartridge 132 may fall within the range of from about 31 μm to about 559 μm, and in some embodiments, from about 76 μm to about 360 μm. Jet injection outcomes may be described by changes in the skin surface, penetration depth, and dispersion shape created by the jet inside the skin 220. The dispersion pattern and penetration depth may greatly effect absorption of the medicament 158 and injection-associated pain. Several factors which may affect drug dispersion and penetration characteristics may include the jet profile, physical or mechanical properties of the medicament 158 and the skin 220 into or beneath which the medicament 158 is injected.

The jet profile of the medicament 158 as it is forcefully expelled or discharged from the injection nozzle 134 of the medicament cartridge 132 may depend on such factors as the nozzle diameter, jet velocity or driving force, and the volume of medicament 158 injected. For example, at a fixed jet velocity and volume, the penetration depth may increase as the nozzle diameter increases. The shape of dispersion may vary according to the nozzle size and jet velocity as well, forming a lower hemisphere, a sphere, an upper hemisphere, or a near-cylinder as the nozzle size and jet velocity increase. This is because the flow of the medicament 158 inside the skin 220 begins at the point source near the center of the sphere. If penetration of the medicament 158 ends inside the skin 220 and dispersion occurs completely within the skin 220, then a sphere or part of a sphere would form. On the other hand, if the fluid penetrates the entire skin thickness, no point source is formed and the dispersion shape would resemble a cylinder.

In some embodiments, the injector CPU 102 of the medicament injector 101 may be pre-programmed to inject the required quantity, volume, and/or number of injection dosage units of the medicament 158 at each physical injection target 222. The quantity, volume, and/or number of injection dosage units of the medicament 158 may be the same for all physical injection targets 222 or may differ depending on the area of the face 219 on which the physical injection target 222 appears. Additionally or alternatively, the injector CPU 102 may be configured to determine the quantity, volume, and/or number of injection dosage unit(s) of the medicament 158 for each physical injection target 222 via user input through the user interface 104. In some embodiments, the injector CPU 102 may be configured to utilize artificial intelligence (AI) to assess and formulate the required quantity, volume, and/or number of injection dosage units of the medicament 158 for each physical injection target 222 for subsequent injection procedures, which in some implementations may be responsive to input via the facial scanner 105 and/or the injector camera 106 of the medicament injector 101 or the facial scanner 185 and/or the subsystem camera 187 of the treatment management subsystem 180. In some embodiments, the injector CPU 102 may be configured to access data files 197 or receive real-time input from the remote healthcare provider 198 (FIG. 1), for instance via the external data processing/storage subsystem 196 and communication network 194, to determine the quantity, volume, and/or number of injection dosage units of the medicament 158 for each physical injection target 222.

In some applications of the medicament injection system 100, the medicament injector 101 and other aspects of the system may be configured to limit if not eliminate the risk of adverse events, facilitating use of the injector by both health-care professionals as well as non-professionals. Botulinum toxin triggers a neuromuscular blocking effect. It acts by binding presynaptically to high-affinity recognition sites on cholinergic nerve terminals blocking presynaptic release of the neurotransmitter (acetylcholine) at the neuromuscular junction. Adverse events can potentially occur when the injected botulinum toxin diffuses to nearby, un-injected muscles, which can result in muscle weakening away from the intended site. Accuracy in location of injection sites (distance of injection nozzle from target muscle, target nerve terminals), injection volume, injection dose, and injection method (including e.g. nozzle characteristics, injection speed, etc.) are all factors that can affect diffusion that can potentially lead to adverse events. Higher injection volumes and dosages can result in greater levels of diffusion. In some instances, higher injection volumes may cause greater diffusion levels that may result in a diminution in duration and magnitude of the desired effect. Higher injection speeds can also cause tissue damage, which may decrease local uptake of the medicament and increase diffusion. The medicament injection systems 100 described herein are designed to limit if not eliminate undesired diffusion to tissues for which no effect is desired.

As described elsewhere in this patent, the medicament injector and systems described herein are configured to allow inexpert operators to accurately and repeatedly inject medicament at precise locations, precise volumes, precise dosages, precise concentrations, and at precise speeds such that diffusion of the injected medicament can be accurately predicted and controlled. The medicament injector standardizes injection volume. The medicament injector has a known nozzle geometry and nozzle diameter associated with a mechanical or electro-mechanical actuator such that a precise and controlled volume of medicament can be delivered at a precise and controlled speed when injected. The injector's use of medicament cartridges (whether pre-filled or automatically reconstituted by the medicament injector) mean that dosage can be precisely known and controlled.

Moreover, as discussed elsewhere in this patent, the medicament injector produces a controlled medicament injection jet of precise geometry and characteristics. Jet injection provides for very uniform dispersion of medicament, and is in sharp contrast to manually actuated injector which does not provide uniform dispersion and is heavily operator dependent in the characteristics of the injection stream. Jet injection also provides for uniformity in injection depth. Standardized volume and dose injected at standardized depth results in even distribution into the muscle and hence even distribution at the pre-synaptic cholinergic nerve terminals. This results in improved efficacy with less diffusion issues related to adverse events. By removing operator error this also allows health professionals and non-health professionals to inject botulinum toxin with a lower learning curve and improved efficacy. Moreover, as also discussed in depth in this patent, implementation of medicament injectors and systems allow for precise location of injection sites, reducing if not eliminating human error in identifying the correct muscle groups to inject. This further reduces issues associated with medicament diffusing into undesired muscles and further facilitates easing use of the injectors and systems by health care professionals as well as non-health care professionals.

In some applications of the medicament injection system 100, the treatment management subsystem 180 may facilitate guided self-injection of botulinum toxin medicament 158 by a patient 218 into one or multiple physical injection targets 222 (FIG. 15) in the skin 220 on the face 219 of a patient 218 using the medicament injector 101. In other applications, the injection procedures may be implemented by an attending healthcare professional or an untrained assisting person. Accordingly, a medicament cartridge 132 having the predetermined quantity of the medicament 158 may be deployed in place on the injector housing 120 of the medicament injector 101. The medicament injector 101 may be turned on by manipulation of the appropriate control feature or features on the user interface 104. The injector CPU 102 may illuminate the power LED 129 on the injector housing 120 to indicate the “ON” status of the medicament injector 101. In some applications, the safety mechanism 151 for the medicament cartridge 132 may be deployed from the safety position illustrated in FIGS. 10 and 12 to the primed position illustrated in FIGS. 11 and 13.

In some implementations of self-injection procedures, the patient 218 may utilize the fingerprint scanner 140 (FIGS. 10-13), the facial recognition capability via the facial scanner 185 of the treatment management subsystem 180, the facial scanner 105 of the medicament injector 101, and/or input a password or passcode into the user interface 104 to unlock the medicament injection 101 for use. Alternatively, the patient 218 may input the password or passcode into the subsystem CPU 181 of the treatment management subsystem 180. In still other alternatives, the medicament injector is not locked and no identification or password is necessary.

The facial scanner 185 on the treatment management system 180 may be operated to obtain a 3-dimensional facial scan of the patient's face 219. The subsystem CPU 181 may responsively generate and present the facial injection map 231 having the facial image 232 of the patient 218 on the display 183 of the treatment management system 180, as illustrated in FIG. 20. Accordingly, based on the facial scan, the subsystem CPU 181 may determine which area or areas of the patient's face 219 require an injection and the quantity, volume, and/or number of injection dosage units of the medicament 158 for the injection. Based on that determination, the subsystem CPU 181 may superimpose on the facial image 232 the virtual injection target or targets 236 which correspond to the respective physical injection targets 222 (FIG. 15) on the patient's face 219 or other area requiring injection to form the facial injection map 231. For example and without limitation, as illustrated in FIG. 20, the virtual injection targets 236 may be located at various intended points of injection on the areas of the facial image 232 which correspond to the glabella 233, the forehead 234, and/or the temples 235 on the patient's face 219. In some applications, the injector CPU 102 of the medicament injector 105, alone or in combination with the subsystem CPU 181 of the treatment management subsystem 180, may perform these and other functions described herein responsive to input from the facial scanner 105.

The facial scanner 185 may be implemented in a variety of ways based on known technologies or in similar fashions to known technologies. As just one example, the facial scanner may be a 3D scanner including a pair of spaced apart cameras angled towards one another and a light source positioned between the cameras. Other examples use a laser and a camera to generate a point cloud that can be assembled into a three dimensional model. These and other implementations may utilize stereo vision and photogrammetry, structured light scanning, time of flight lasers, or other techniques for obtaining the 3-D facial scan of the treatment area, which may take the form of a single image or a video model.

In some applications, the subsystem CPU 181 may transmit the quantity, volume, and/or number of injection dosage units of the medicament 158 for each physical injection target 222 to the injector CPU 102 of the medicament injector 101. The quantity, volume, and/or number of injection dosage units of the medicament 158 to be injected at each physical injection target 222 may be indicated on the display 183 of the treatment management subsystem 180 and/or on the touchscreen 127 of the medicament injector 101. For example and without limitation, the subsystem CPU 181 may numerically indicate the quantity, volume, and/or number of injection dosage unit(s) of the medicament 158 to be injected at each physical injection target 222 at or adjacent to the corresponding virtual injection target 236 on the facial image 232 (FIG. 20) of the facial injection map 231 which appears on the display 183.

The patient 218, attending healthcare provider or untrained assisting person, hereinafter patient 218, may grasp the handle portion 122 of the injector housing 120 and hold the medicament injector 101 with one hand while using the virtual injection targets 236 which appear on the facial injection map 231 as a guide to the locations and positions of the respective physical injection targets 222 on the patient's face 219. As the patient 218 next aims the front housing surface 125 of the main housing portion 121 toward the skin 220, the targeting LED 110 (FIG. 3) on the medicament injector 101 may direct the targeting LED beam 221 (FIG. 15) toward and against one or more of the physical injection targets 222 on the patient's face 219. Additionally or alternatively, the targeting LED beam 221 emitted by the targeting LED 110 may impinge on the skin 220 to form a ring shape which surrounds or encircles the physical injection target or targets 222. As the cartridge leveling disk 137 of the medicament cartridge 132 contacts the skin 220 at the illuminated physical injection target 222, the angle sensor 108 on the medicament injector 101 may emit an angle sensor beam 230 which impinges on the skin 220, as illustrated in FIGS. 21 and 22. Based on the angle of impingement of the angle sensor beam 230, the angle sensor 108 may transmit a signal to the injector CPU 102 which enables the injector CPU 102 to determine whether the longitudinal axis of the medicament cartridge 132 is disposed at the optimal injection angle 226 with respect to the surface of the skin 220. If the injection angle 226 is not optimal, as illustrated in FIG. 21, then the injector CPU 102 may cause the injection angle indicator 111 (FIG. 3) to notify the incorrect injection angle 226 to the patient 218 audibly and/or visually. For example and without limitation, in some applications, the injection angle indicator 111 may include a light which encircles the main housing portion 121 of the injector housing 120 and illuminates red if the injection angle 226 is not optimum. The patient 218 may then correct the orientation of the injector housing 120 until the injection angle 226 is optimum (90 degrees or perpendicular), as illustrated in FIG. 22. The injector CPU 226 may responsively illuminate the injection angle indicator 111 in the second color (such as green) to indicate that the medicament cartridge 132 is oriented at the correct injection angle 226 for injection. In some embodiments, the plane of the cartridge leveling disk 137 may be fixed at the optimum injection angle (90 degrees) with respect to the longitudinal axis of the medicament cartridge 132 to ensure the optimum injection angle 226 upon uniform engagement of the cartridge leveling disk 137 with the patient's skin 220.

After the optimum injection angle 226 is attained, the patient 218 may utilize the user interface 104 of the medicament injector 101 to facilitate forceful injection of the selected quantity, volume, and/or number of injection dosage units of the medicament 158 from the main cartridge portion 133 through the injection nozzle 134 and the injection opening 138 in the cartridge leveling disk 137, respectively, into or beneath the skin 220 on the patient's face 219 at the selected physical injection target 222. In some applications, this may be accomplished by a single depression or actuation of the trigger 128 of the user interface 104. Subsequent release of the trigger 128 may facilitate retraction of the actuator piston 146 (FIGS. 10 and 11) to the retracted actuator piston position and return of the plunger piston 149 to the retracted plunger piston position via the plunger piston return spring 150, preparatory to another injection.

The medicament injector 101 may then be moved away from the physical injection target 222 at which the injection was made to disengage the cartridge leveling disk 137 from the skin 220. As the patient 218 continues to use the virtual injection targets 236 on the facial injection map 231 as a guide, the patient 218 may move the medicament injector 101 to a different physical injection target 222 to facilitate injection of the medicament 158 into or beneath the skin 220 at that physical injection target 222 in like manner. The foregoing procedure may be repeated until each physical injection target 222 which corresponds to a virtual injection target 236 on the facial image 232 of the facial injection map 231 (FIG. 20) is treated. In some applications, the quantity, volume, and/or number of injection dosage units of the medicament 158 injected at each physical injection target 222 for each treatment session may be stored by the injector CPU 102 of the medicament injector 101 and/or by the subsystem CPU 181 of the treatment management subsystem 180. This information may be utilized to formulate a treatment plan for the subsequent treatment session(s). In some applications, the subsystem CPU 181 and/or the injector CPU 102 may collect treatment data and utilize artificial intelligence (AI) to identify treatment trends or patterns which can be used to formulate the frequency of treatment sessions as well as to adjust the quantity, volume, and/or number of injection dosage units of the medicament 158 which may be necessary for injection at each physical injection target 222 in subsequent treatment sessions.

In some applications, the subsystem camera 187 of the treatment management subsystem 180 and/or the injector camera 106 of the medicament injector 101 may capture high-resolution still and/or video images of the patient's face 219 before, during and/or after the treatment session. For example and without limitation, in some applications, the subsystem camera 187 may capture one or more general images, whereas the injector camera 106 may capture one or more close-up images of the patient's face 219. These images may be stored as video and/or still image data files 197 which can be accessed by the remote healthcare provider 198. The remote healthcare provider 198 may review the recorded images and/or view the images in real time to provide instruction, guidance, advice or feedback to the patient 218 in carrying out current or subsequent injection procedures. The information provided by the remote healthcare provider 198 may be used to determine such parameters as the frequency of treatment sessions; the size, volume, and/or quantity of medicament 158 in each injection dosage unit; and the number of injection dosage units of the medicament 158 which may be necessary for injection at each injection site. The microphone 189 and the speaker 191 of the treatment management subsystem 180 and/or the microphone 115 and the speaker 118 of the medicament injector 101 may facilitate real-time communication between the remote healthcare provider 198 and the patient 218 or untrained assisting person before, during and/or after the treatment session.

Throughout each treatment session, the medication sensor(s) 112 (FIG. 3) may monitor the quantity, volume, or number of injection dosage units of the medicament 158 which remain(s) in the medicament cartridge 132. The injector CPU 102 may display the measured remaining amount of the medicament 158 and display the amount on the touchscreen 127 of the user interface 104 on the medicament injector 101 and/or on the display 183 of the treatment management subsystem 180. As needed, the patient 218 may remove the empty medicament cartridge 132 from the injector housing 120 of the medicament injector 101 and deploy a replacement medicament cartridge 132 in its place for continuance of the treatment session.

In some applications, the injector CPU 102 may determine whether each injection of the medicament 158 was a successful injection after the injection is completed. This may be accomplished by determining whether and to what extent “splashback” of the medicament 158 may have occurred during the injection. The injector CPU 102 may indicate whether the injection was a success on the touchscreen 127 of the user interface 104 on the medicament injector 101, the display 183 of the treatment management subsystem 180 and/or in any other suitable manner.

In some applications, the patient 218 may program reminders for upcoming treatment sessions via the user input 184. The subsystem CPU 181 may provide periodic reminders of the injection procedures or treatment sessions for the patient 218 between sessions audibly and/or visually on the display 183 of the treatment management subsystem 180 and/or the touchscreen 127 of the user interface 104 on the medicament injector 101.

The communication network 194 may include any network or combination of networks which is/are configured to facilitate the data communication tasks described herein. For example and without limitation, in some embodiments, the communication network 194 may include the Internet, Ethernet and/or one or more separate public, semi-public, and/or private communication networks. The subsystem CPU 181 may be provided with the appropriate platform(s) to create and manage applications, run analytics, and store and secure the data as the data files 197. The subsystem CPU 181 of the treatment management subsystem 180 and/or the injector CPU 102 of the medicament injector 101 may be configured to utilize cybersecurity and encryption technology to encrypt the communications via the communication network 194 and/or the direct communications between the medicament injector 101 and the treatment management subsystem 180.

In some embodiments, the system 100 may utilize the Internet of Things (IoT) or Internet of Medical Things (IoMT) technology to facilitate implementation of the various functions of the medicament injector 101. IoT and IoMT technology describes physical objects (or groups of such objects) with sensors, processing ability, software and other technologies that connect and exchange data with other devices and systems over the Internet or other communications network. The subsystem CPU 181 may be configured to facilitate the injection procedures in a treatment session as well as data collection and analysis for research and monitoring utilizing IoT and/or IoMT technology. The sensors 107 (FIG. 3) which interface with the injector CPU 102 of the medicament injector 101 may include smart or intelligent sensors having the capability to collect, process, transfer, and analyze information related to the quantity, volume, and/or number of injection dosage units of the medicament 158 which the medicament injector 101 injects into the patient 218 in each injection procedure. The intelligent sensors may additionally or alternatively have the capability to collect, process, transfer, and analyze information related to the appropriate number, scheduling and/or frequency of treatment sessions for the patient 218 and/or the treatment areas on the patient 218 which may require an injection procedure for each treatment session and store the information as data files 197 (FIG. 4), for the purpose of formulating or planning subsequent treatment sessions. These data files 197 may be made available to the attending healthcare provider or untrained assisting person and/or to the remote healthcare provider 198 for instruction, guidance, advice, or feedback in carrying out current and/or subsequent injection procedures, as was heretofore described. In some embodiments, the system 100 may utilize IoT and/or IoMT technology married with AI technology to improve the predictive-maintenance capability of the system 100.

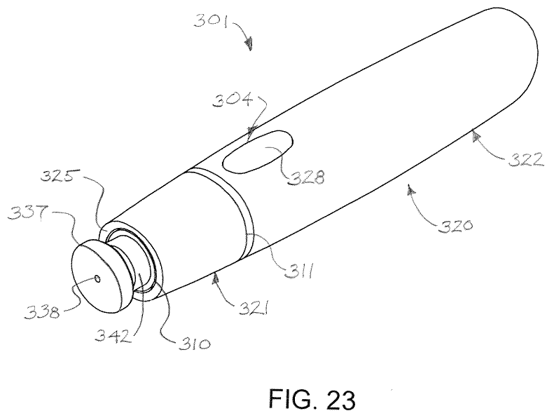

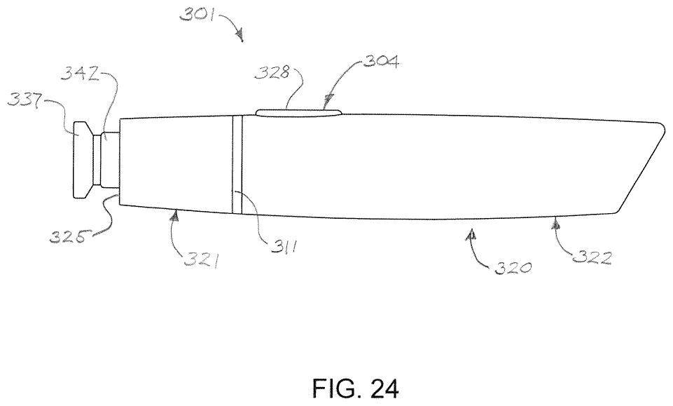

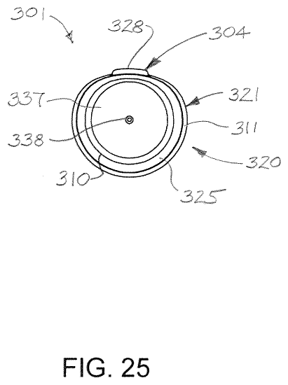

### Alternative Medicament Injector—FIGS. 23-29

Referring next to FIGS. 23-29 of the drawings, an alternative illustrative gas-actuated embodiment of the medicament injector which is suitable for implementation of the medicament injection systems is generally indicated by reference numeral 301. The medicament injector 301 may be suitable for use with the medicament injection system 100 which was heretofore described with respect to FIGS. 1-22. Unless otherwise indicated, elements of the medicament injector 301 which are structurally and/or functionally analogous to the respective elements of the medicament injector 101 that was heretofore described with respect to FIGS. 1-22 are designated by the same respective reference numerals in the 301-399 series in FIGS. 23-29. Accordingly, to the extent which is applicable, the same description which was heretofore described with respect to the medicament injector 101 is incorporated by reference herein in its entirety herein with respect to the medicament injector 301.

The medicament injector 301 may have an elongated injector housing 320. In some embodiments, the injector housing 320 may be fabricated in separable housing subunits (not illustrated) to facilitate access to the interior of the injector housing 320. The housing subunits may be detachably attachable to each other via multiple tabs and interfacing tab openings on the respective subunits, for example and without limitation. The injector housing 320 of the medicament injector 301 may be straight, with the longitudinal axis of the main housing portion 321 being coaxial with the longitudinal axis of the handle portion 322, as illustrated. In other embodiments, the longitudinal axis of the handle portion 322 may be disposed at an obtuse housing angle 124 (FIG. 6) with respect to the longitudinal axis of the main housing portion 321.

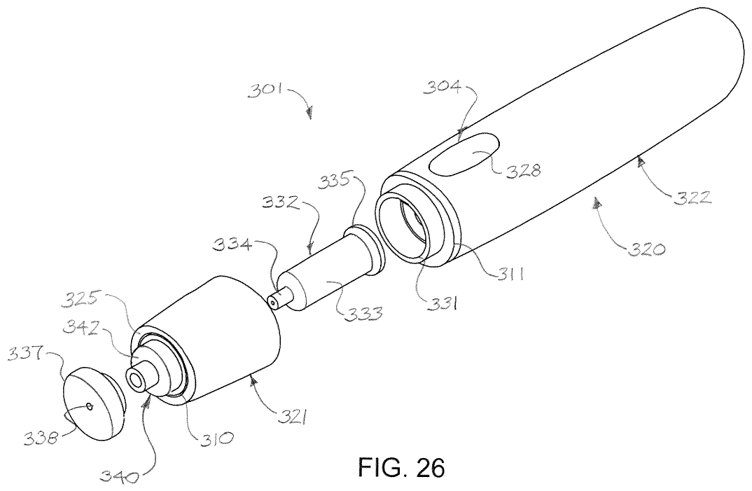

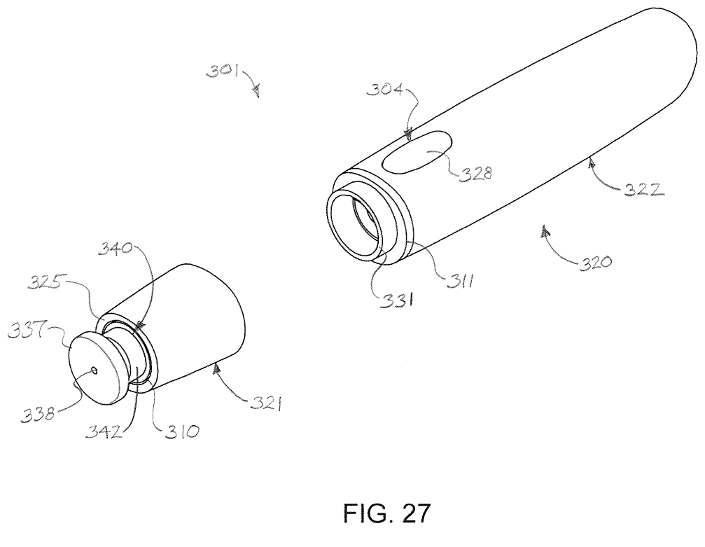

In some embodiments, the main housing portion 321 may detachably engage the handle housing portion 322 of the injector housing 320 according to the knowledge of those skilled in the art. Accordingly, as illustrated in FIGS. 26 and 27, in some embodiments, a housing nipple 331 may protrude from the front or forward end of the handle portion 322 of the injector housing 320. The main housing portion 321 may detachably engage the housing nipple 331 via a threaded connection, interference fit or the like.

The injection angle indicator 311 may include at least one circular angle indicator light which encircles the injector housing 320 of the medicament injector 301, as illustrated. The angle indicator light 311 may illuminate a first color (such as red) when the injection angle 226 is not optimal (FIG. 21) and a second color (such as green) when the injection angle 226 is the optimal value (FIG. 22). The angle indicator light 311 may be provided at any point, position, or location along the length of the injector housing 320 which enables the patient 218 to visualize the angle indicator light 311 as the cartridge leveling disk 337 is deployed against the skin 220 such as on the face 219 of the patient 218 preparatory to the injection. For example and without limitation, in some embodiments, the injection angle indicator light 311 may be provided at the interface between the main housing portion 321 and the handle portion 322 of the injector housing 320, as illustrated. The main housing portion 321 may progressively taper or decrease from the injection angle indicator 311 to the front housing surface 325 to render the injection indicator light 311 visible to the patient 218 before and throughout the injection procedure.

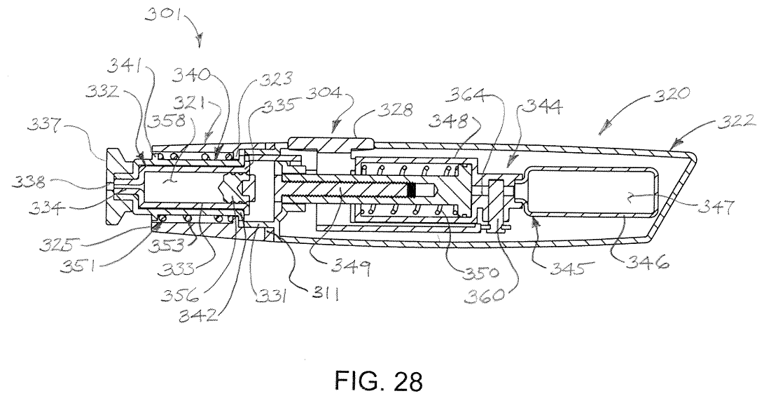

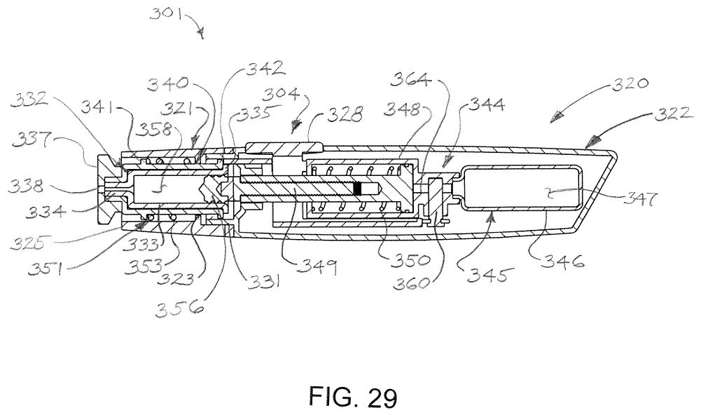

As illustrated in FIGS. 28 and 29, in some embodiments, the medicament injection assembly 344 of the medicament injector 301 may include a gas actuator 345. The gas actuator 345 may include at least one gas cartridge 346. The gas cartridge 346 may be configured to contain a supply of pressurized gas 347. The pressurized gas 347 may include compressed air, carbon dioxide, and/or other compressed gas or combination of compressed gases suitable for the purpose of the gas actuator 345 as described herein. A plunger piston housing 348 may be provided in the injector housing 320, forwardly of the gas cartridge 346. The gas cartridge 346 may be disposed or disposable in fluid communication with the plunger piston housing 348. In some embodiments, a gas passage 364 may establish fluid communication between the gas cartridge 346 and the plunger piston housing 348. The gas cartridge 346 may be configured to sealingly and detachably engage the plunger piston housing 348.

A plunger piston 349 may be slidably disposed between a retracted plunger piston position (illustrated in FIGS. 28 and 29) and an extended plunger piston position in the plunger piston housing 348. The gas cartridge 346 of the gas actuator 345 may be disposed in fluid communication with the plunger piston housing 348. As it flows from the gas cartridge 346 through the gas passage 364 into the plunger piston housing 348, the pressurized gas 347 may apply forward pressure against the plunger piston 349 such that the plunger piston 349 is deployed from the retracted plunger piston position toward the extended plunger piston position, against the cartridge plunger 356 of the medicament cartridge 332. Continued extension of the plunger piston 349 to the extended plunger piston position may cause the plunger piston 349 to advance the cartridge plunger 356 forwardly in the main cartridge portion 333 of the medicament cartridge 332 such that the cartridge plunger 356 forcefully expels at least a predetermined quantity, volume, and/or number of injection dosage units of the medicament 358 from the main cartridge portion 333 through the injection nozzle 334 and the injection opening 338 in the cartridge leveling disk 337, respectively.

A gas release valve 360 may be disposed at the gas passage 364 between the plunger piston housing 348 and the gas cartridge 346 of the gas actuator 345. The gas release valve 360 may be selectively positional in a closed valve position in which the gas release valve 360 closes the gas passage 364, as illustrated in FIGS. 28 and 29, and an open valve position in which the gas release valve 360 opens the gas passage 364. The trigger 328 and/or other control feature or features of the user interface 304 may operably interface with the gas release valve 360 for selective deployment of the gas release valve 360 in the closed and open valve positions. Opening of the gas release valve 360 may facilitate flow of the compressed gas 347 from the gas cartridge 346 through the gas passage 364 into the plunger piston housing 348 for forceful deployment of the plunger piston 349 from the retracted plunger position to the extended plunger piston position.

In some embodiments, a plunger piston return spring 350 may be provided in the plunger piston housing 348. The plunger piston return spring 350 may engage and normally bias the plunger piston 349 in the retracted plunger piston position in the plunger piston housing 348. Accordingly, as the flowing pressurized gas 347 in the gas cartridge 346 of the gas actuator 345 deploys the plunger piston 349 from the retracted plunger piston position to the extended plunger piston position in the plunger piston housing 348, the plunger piston 349 may advance in the plunger piston housing 348 against the bias imparted by the plunger return spring 350. Upon subsequent release of the pressurized gas 347 from the plunger piston housing 348, via detachment and removal of the empty gas cartridge 346 from the plunger piston housing 348, the plunger piston return spring 350 may return the plunger piston 349 from the extended plunger piston position back to the retracted plunger piston position in the plunger piston housing 348. The gas release valve 360 may subsequently be returned to the closed valve position and a replacement gas cartridge 346 deployed in place on the plunger piston housing 348 preparatory to another deployment of the plunger piston 349 from the retracted plunger piston position to the extended plunger piston position, via actuation of the trigger 328.

As further illustrated in FIGS. 26-29, in some embodiments, the safety mechanism 351 for the medicament cartridge 332 of the medicament injector 301 may include a cartridge housing 340. The cartridge housing 340 may have the same shape as the medicament cartridge 332 and may be suitably sized and configured to contain the medicament cartridge 332 in the injector housing 320. As illustrated in FIGS. 28 and 29, the cartridge housing 340 may include an exterior front cartridge housing flange 341 and an exterior rear cartridge housing flange 342, for purposes which will be hereinafter described. The cartridge housing 340, with the medicament cartridge 332 contained therein, may be slidably disposed in the main housing portion 321 of the injector housing 320. The cartridge housing 340 may be selectively deployable in a safety position illustrated in FIG. 28 and a primed position illustrated in FIG. 29 for use of the medicament injector 101. As illustrated in FIG. 28, in the safety position, the cartridge housing 340 and the medicament cartridge 332 contained therein may protrude beyond the front housing surface 325 of the main housing portion 321, exterior to the main housing portion 321, such that the plunger piston 349 disengages the cartridge plunger 356 and a gap (not numbered) exists therebetween. In the primed position, illustrated in FIG. 29, the cartridge housing 340 may be retracted into the main housing portion 321 such that the gap between the plunger piston 349 and the cartridge plunger 356 is closed and the plunger piston 349 engages the cartridge plunger 356. A main cartridge portion flange 323 may extend from the interior surface into the interior of the main housing portion 321 of the injector housing 320. The rear cartridge housing flange 342 of the cartridge housing 340 may engage the main cartridge portion flange 323 in the safety position (FIG. 28) and may disengage the main cartridge portion flange 323 in the primed position (FIG. 29) of the cartridge housing 340 and the medicament cartridge 332.

As illustrated in FIGS. 26-29, in some embodiments, the cartridge flange 335 may be disposed at or adjacent to the rearmost end or edge of the main cartridge portion 333 of the medicament cartridge 332. When the medicament cartridge 332 is deployed in place in the injector housing 320, the cartridge flange 335 of the medicament cartridge 332 may be disposed between the housing nipple 331 of the handle portion 322 and the rear cartridge housing flange 342 of the cartridge housing 340.

As further illustrated in FIGS. 28 and 29, a safety spring 353 may be provided in the main housing portion 321 between and in engagement with the main housing portion flange 323 and the front cartridge housing flange 341 on the cartridge housing 340. The safety spring 353 may normally bias the cartridge housing 340 and the medicament cartridge 332 therein in the safety position illustrated in FIG. 28, with the gap between the plunger piston 349 and the cartridge plunger 356. As the cartridge housing 340 with the medicament cartridge 332 is deployed from the safety position to the primed position preparatory to the injection procedures, against the bias imparted by the safety spring 353, the gap between the plunger piston 349 and the cartridge plunger 356 may be closed until the plunger piston 349 engages the cartridge plunger 356, as illustrated in FIG. 29. The cartridge housing 340 may engage the main housing portion 321 or may engage a catch (not illustrated) provided in the interior of the main housing portion 321 to maintain the medicament cartridge 332 in the primed position. In some embodiments, the medicament cartridge 332 may be deployed from the safety position to the primed position such as by application of manual pressure to the cartridge housing 340 in the rearward direction, or towards the main housing portion 321. In other embodiments, a lever or other mechanism (not illustrated) may facilitate selective deployment of the cartridge housing 340 and medicament cartridge 332 from the safety position to the primed position.

Application of the medicament injector 301 may be as was heretofore described with respect to the medicament injector 101 in FIGS. 1-22. Preparatory to an injection procedure, the gas release valve 360 may be deployed in the closed valve position, by actuation of the trigger 328 of the user interface 304. The gas cartridge 346, having a full supply of the compressed gas 347, may be connected to the plunger piston housing 348. This may be accomplished by detaching the housing subunits of the injector housing 320 from each other to open and access the interior of the injector housing 320, attaching the gas cartridge 346, and reattaching the housing subunits to close the injector housing 320. As illustrated in FIGS. 26 and 27, the medicament cartridge 332 may be deployed in the injector housing 320 by removing the main housing portion 321 from the handle portion 322 and inserting the medicament cartridge 332 into the rear end of the cartridge housing 340 until the cartridge flange 335 engages the rear cartridge housing flange 342 of the cartridge housing 340. The main housing portion 321 may then be replaced on the handle portion 322 of the injector housing 320 by engagement of the main housing portion 321 with the housing nipple 331, as was heretofore described. The cartridge housing 340 and medicament cartridge 332 therein may be deployed from the safety position (FIG. 28) to the primed position (FIG. 29).

In deployment of the medicament injector 301 against the skin 220 on the face 219 of the patient 218 preparatory to injecting the medicament 358 into or beneath the skin 220 for each injection procedure in a treatment session, the cartridge leveling disk 337 may uniformly engage the skin 220 to ensure that the injection angle 226 (FIGS. 21 and 22) remains at the correct or optimum value for the injection. The injection angle indicator 311 may additionally provide a visual and/or audible indication to the patient 218 that the desired injection angle 226 has been achieved.

Actuation of the trigger 328 and/or other control feature(s) of the user interface 304 may facilitate flow of the pressurized gas 347 from the gas cartridge 346 of the gas actuator 345 through the gas passage 364 into the plunger piston housing 348 by opening of the gas release valve 360. Accordingly, the flowing pressurized gas 347 may forcefully deploy the plunger piston 349 from the retracted plunger piston position to the extended plunger piston position against the bias imparted by the plunger piston return spring 350. The advancing plunger piston 349 may advance the cartridge plunger 356 in the main cartridge portion 333 of the medicament cartridge 332 such that the cartridge plunger 356 discharges the predetermined quantity, volume, and/or injection dosage units of the medicament 358 for the injection procedure. As deemed necessary during the treatment session, a replacement medicament cartridge 332 and/or a replacement gas cartridge 346, having a full supply of pressurized gas 347, may be deployed in place in the injector housing 320 for subsequent injections, as was heretofore described.

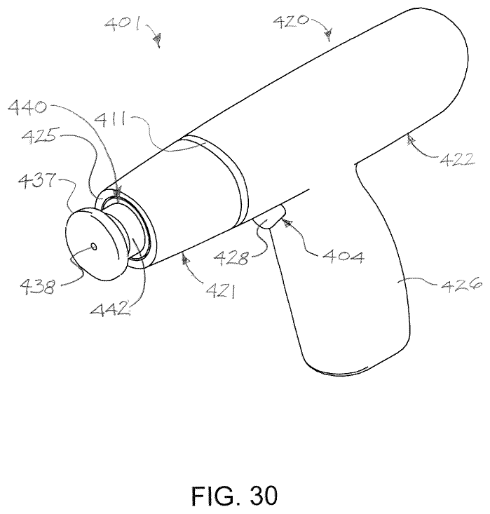

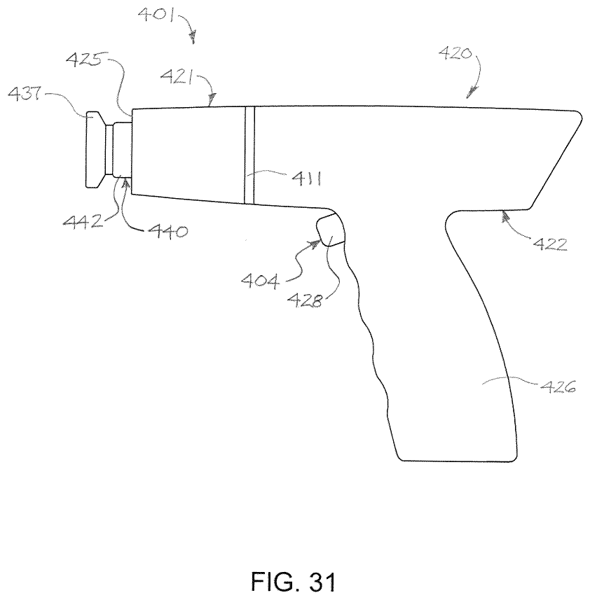

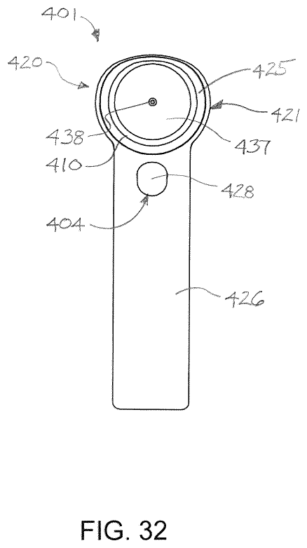

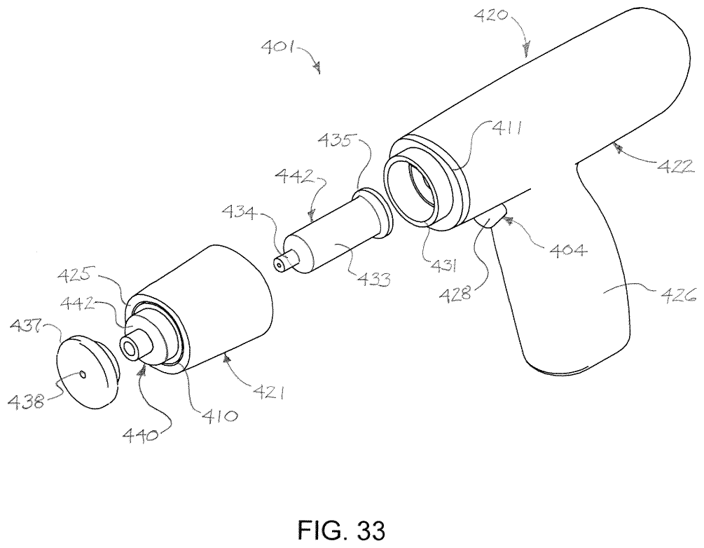

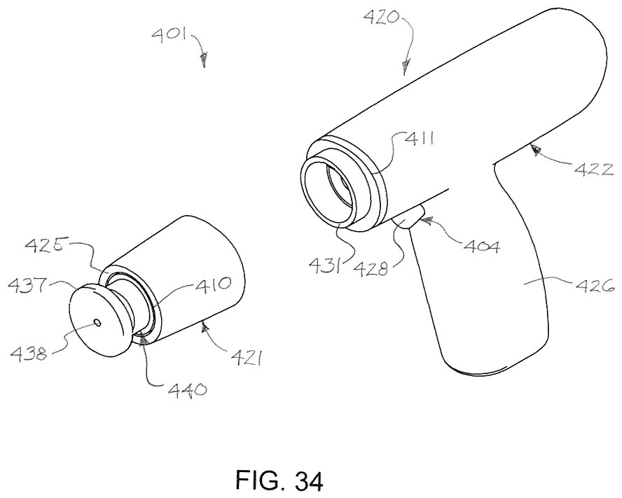

### Alternative Medicament Injector—FIGS. 30-36

Referring next to FIGS. 30-36 of the drawings, another alternative illustrative gas-actuated embodiment of the medicament injector which is suitable for implementation of the medicament injection systems 100 by an attending healthcare provider or untrained assisting person is generally indicated by reference numeral 401. The medicament injector 401 may be suitable for use with the medicament injection system 100 which was heretofore described with respect to FIGS. 1-22. Unless otherwise indicated, elements of the medicament injector 401 which are structurally and/or functionally analogous to the respective elements of the medicament injector 101 that was heretofore described with respect to FIGS. 1-22 and the elements of the medicament injector 301 that was heretofore describe with respect to FIGS. 23-29 are designated by the same respective reference numerals in the 301-399 series in FIGS. 30-36. Accordingly, to the extent which is applicable, the same descriptions which were heretofore described with respect to the medicament injector 101 and with respect to the medicament injector 301 are incorporated by reference herein in their entireties herein with respect to the medicament injector 401.

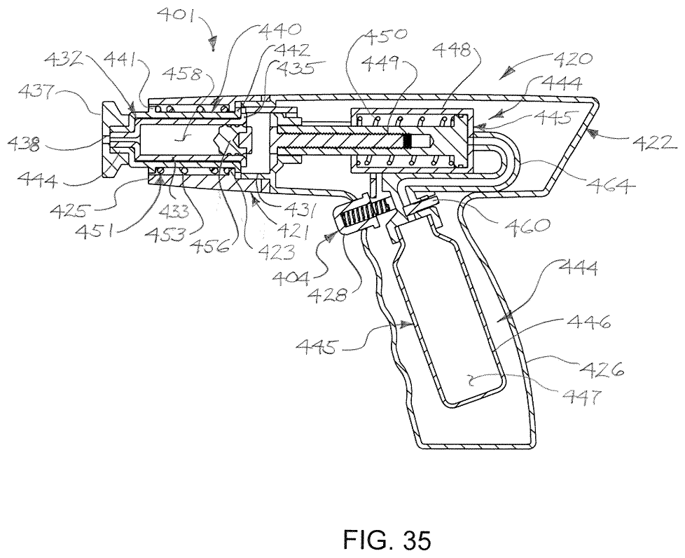

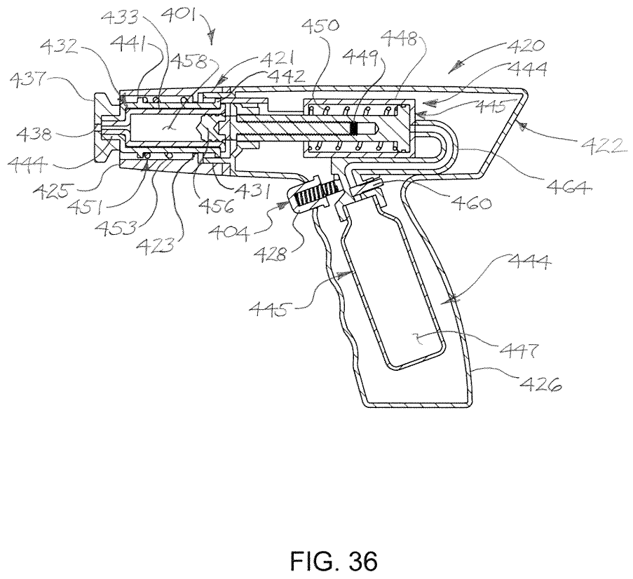

A handle grip 426 may extend from the handle portion 422 on the injector housing 420 of the medicament injector 401. The handle grip 426 may enable the attending healthcare provider or untrained assisting person to grip and hold the medicament injector 401 in a steady manner as the attending healthcare provider or untrained assisting person applies the medicament injector 401 to the skin 220 (FIG. 15) on the face 219 of the patient 218. As illustrated in FIGS. 35 and 36, in some embodiments, the gas cartridge 446 of the gas actuator 445 may be disposed in the handle grip 426. A gas passage 464 may connect the plunger piston housing 448 in fluid communication with the gas cartridge 446.

Application of the medicament injector 401 may be as was heretofore described with respect to the medicament injector 101 in FIGS. 1-22 and the medicament injector 301 in FIGS. 23-29. The attending healthcare provider or untrained assisting person may grasp the handle grip 426 of the injector housing 420 with one hand for accurate and steady deployment of the medicament injector 401 against the skin 220 on the face 219 of the patient 218 preparatory to and during injection of the medicament 458 into or beneath the skin 220 for each injection procedure in a treatment session. Actuation of the trigger 428 and/or other control feature(s) of the user interface 404 may facilitate flow of the pressurized gas 447 from the gas cartridge 446 of the gas actuator 445 through the gas passage 464 into the plunger piston housing 448 by opening of the gas release valve 460. The flowing pressurized gas 447 may deploy the plunger piston 449 from the retracted plunger piston position to the extended plunger piston position against the bias imparted by the plunger piston return spring 450. The advancing plunger piston 449 may advance the cartridge plunger 456 in the main cartridge portion 433 of the medicament cartridge 432 such that the cartridge plunger 456 discharges the predetermined quantity, volume, and/or injection dosage units of the medicament 458 from the medicament cartridge 432 for the injection procedure. As deemed necessary during the treatment session, a replacement medicament cartridge 432 and/or a replacement gas cartridge 446, such as having a full supply of the pressurized gas 447, may be deployed in place in the injector housing 420, as was heretofore described.

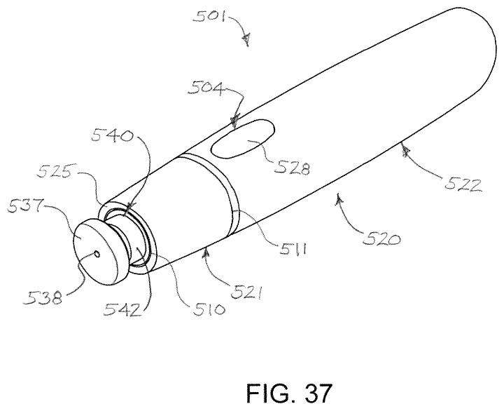

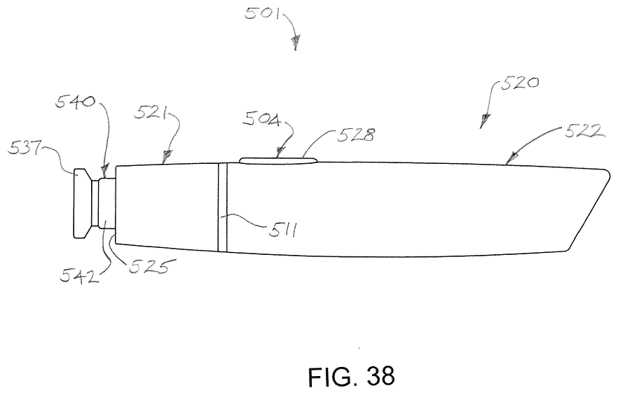

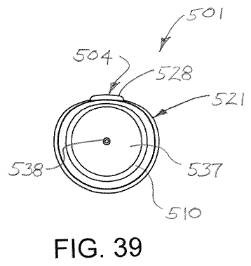

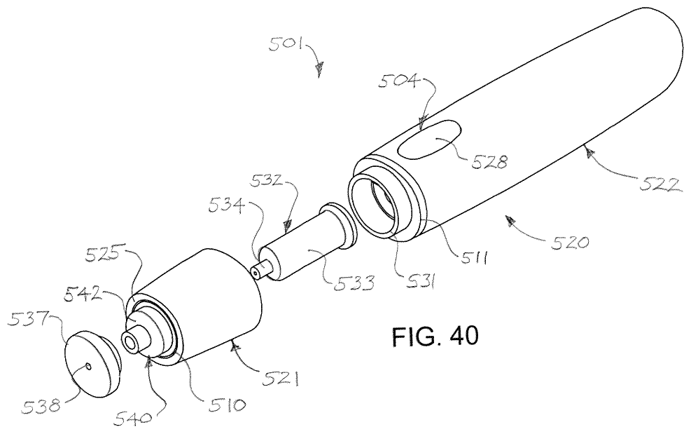

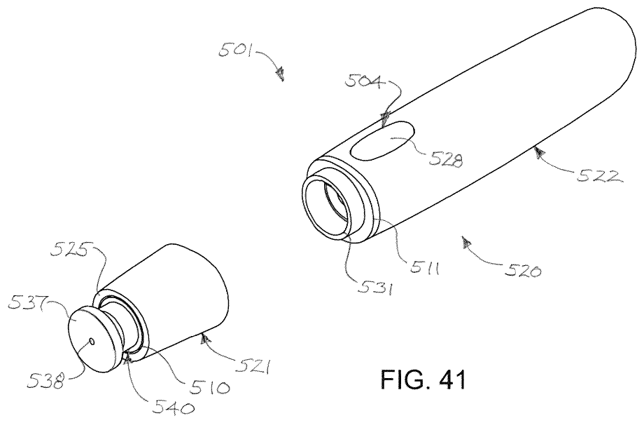

### Alternative Medicament Injector—FIGS. 37-43

Referring next to FIGS. 37-43 of the drawings, an alternative illustrative spring-actuated embodiment of the medicament injector which is suitable for implementation of the medicament injection systems 100 is generally indicated by reference numeral 501. The medicament injector 501 may be suitable for use with the medicament injection system 100 which was heretofore described with respect to FIGS. 1-22. Unless otherwise indicated, elements of the medicament injector 501 which are structurally and/or functionally analogous to the respective elements of the medicament injector 301 that was heretofore described with respect to FIGS. 23-29 are designated by the same respective reference numerals in the 501-599 series in FIGS. 37-43. Accordingly, to the extent which is applicable, the same description which was heretofore described with respect to the medicament injector 301 is incorporated by reference herein in its entirety herein with respect to the medicament injector 501.

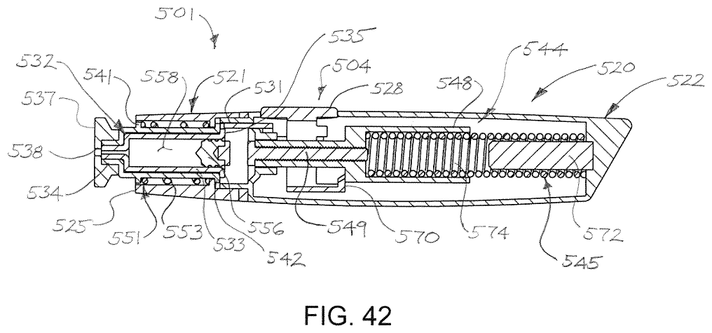

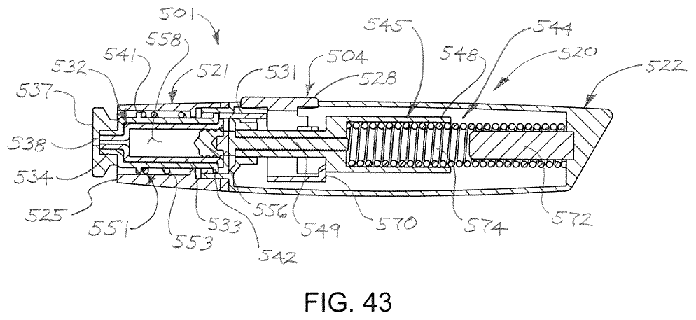

As illustrated in FIGS. 42 and 43, the medicament injection assembly 544 of the medicament injector 501 may include a spring actuator 545. The spring actuator 545 may include a plunger piston housing 548 which moves in concert with the plunger piston 549 as the plunger piston 549 deploys between the retracted plunger piston position and the extended plunger piston position. In some embodiments, the plunger piston housing 548 may be fabricated in one piece with the plunger piston 549.

A plunger piston deployment spring 574 may be disposed in the plunger piston housing 548. The compressed plunger piston deployment spring 574 may operably engage the plunger piston 549 to normally bias the plunger piston 549 toward the extended plunger piston position in the injector housing 520. A spring mount member 572 may be disposed in the injector housing 520. The spring mount member 572 may receive and stabilize the plunger piston deployment spring 574 in the interior of the injector housing 520 as the plunger piston deployment spring 574 extends over the spring mount member 572 and engages the interior surface of the injector housing 520.

A trigger flange 570 may extend from the trigger 528 of the user interface 504. The trigger 528 may be selectively deployable on the injector housing 520 in a piston engaging position and a piston disengaging position. In the piston engaging position illustrated in FIGS. 42 and 43, the trigger flange 570 may be configured to retain the plunger piston housing 548 in the retracted plunger piston position against the spring-extending bias imparted by the plunger deployment spring 574. In the piston disengaging position (not illustrated) of the trigger 528, the trigger flange 570 may be configured to disengage the plunger piston housing 548 to facilitate deployment of the plunger piston housing 548 and the plunger piston 549 from the retracted plunger piston position to the extended plunger piston position responsive to extension of the plunger piston deployment spring 574.

Application of the medicament injector 501 may be as was heretofore described with respect to the medicament injector 101 in FIGS. 1-22 and the medicament injector 301 in FIGS. 23-29. Preparatory to an injection procedure, the trigger 528 may be deployed in the piston engaging position on the injector housing 520 to retain the plunger piston 549 in the retracted plunger piston position illustrated in FIGS. 42 and 43. After deployment of the medicament injector 501 against the skin 220 on the face 219 of the patient 218 preparatory to injecting the medicament 558 into or beneath the skin 220 for each injection procedure in a treatment session, the trigger 528 and/or other control feature(s) of the user interface 304 may be actuated to facilitate disengagement of the trigger flange 570 from the plunger piston housing 548. Accordingly, the initially compressed plunger piston deployment spring 574 may extend to deploy the plunger piston housing 548 and the plunger piston 549 attached thereto from the retracted plunger piston position to the extended plunger piston position. The advancing plunger piston 549 may advance the cartridge plunger 556 in the main cartridge portion 533 of the medicament cartridge 532 such that the cartridge plunger 556 discharges the predetermined quantity, volume, and/or injection dosage units of the medicament 558 from the medicament cartridge 532 for the injection procedure. As deemed necessary during the treatment session, a replacement medicament cartridge 532 may be deployed in place in the injector housing 520.

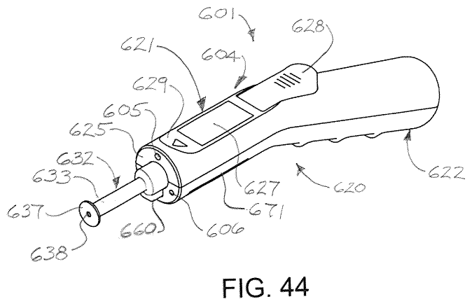

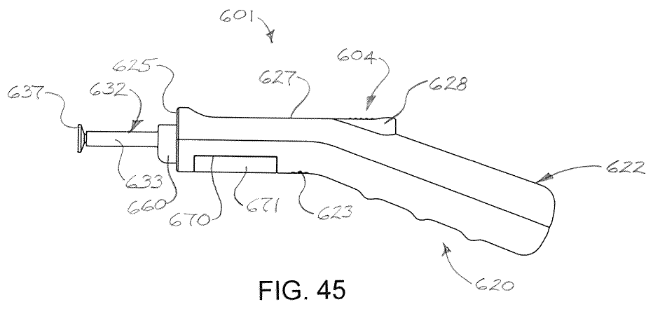

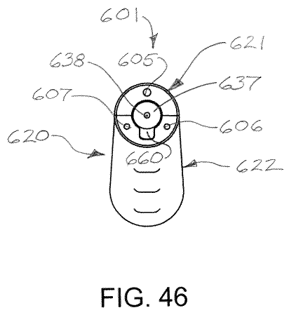

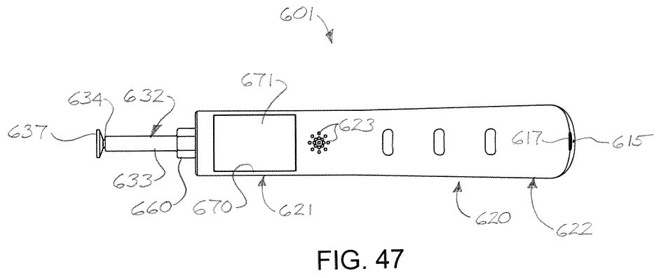

### Alternative Medicament Injector—FIGS. 44-52

Referring next to FIGS. 44-52 of the drawings, an illustrative mixed cartridge embodiment of the medicament injector of the medicament injection systems is generally indicated by reference numeral 601. The mixed cartridge medicament injector 601 may be suitable for use with the medicament injection system 100 which was heretofore described with respect to FIGS. 1-22. Unless otherwise indicated, elements of the medicament injector 601 which are structurally and/or functionally analogous to the respective elements of the medicament injector 101 that was heretofore described with respect to FIGS. 1-22 are designated by the same respective reference numerals in the 601-699 series in FIGS. 44-52. Accordingly, to the extent which is applicable, the same description which was heretofore described with respect to the medicament injector 101 is incorporated by reference herein in its entirety herein with respect to the medicament injector 601.

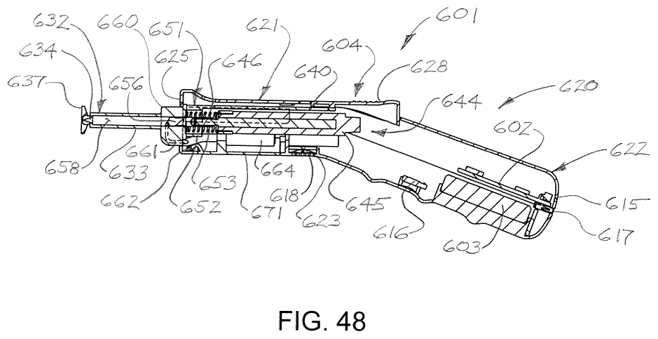

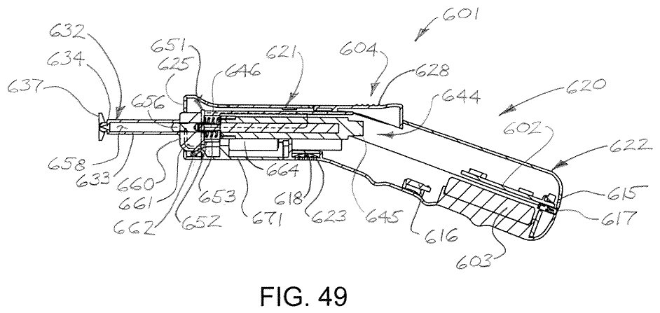

As illustrated in FIGS. 48-52, the injector housing 620 of the medicament injector 601 may include a fluid distribution manifold 660. The main cartridge portion 633 of the medicament cartridge 632 may be configured for detachable confluent attachment to the fluid distribution manifold 660 preparatory to an injection procedure or treatment session. In some embodiments, the fluid distribution manifold 660 may protrude forwardly from the front housing surface 625 of the main housing portion 621. The main cartridge portion 633 of the medicament cartridge 632 may be configured to insert into a cartridge opening (not illustrated) in the fluid distribution manifold 660, as was heretofore described with respect to attachment of the main cartridge portion 133 of the medicament cartridge 132 to the cartridge attachment nipple 130 of the medicament injector 101. As illustrated in FIGS. 48 and 49, the fluid distribution manifold 660 may have an interior distribution manifold passage 661. When the medicament cartridge 632 is attached to the fluid distribution manifold 660, the distribution manifold passage 661 may be disposed in fluid communication with the main cartridge portion 633 of the medicament cartridge 632.

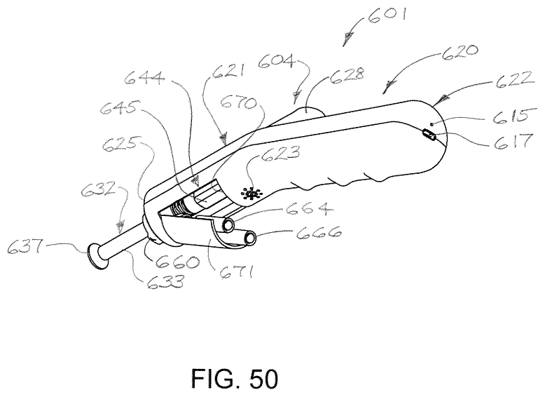

As illustrated in FIG. 50, a cartridge compartment 670 may be provided in the main housing portion 621 of the injector housing 620. A cartridge compartment cover 671 may be configured to detachably engage the main housing portion 621 to facilitate selective opening and closing of the cartridge compartment 670. For example and without limitation, in some embodiments, the cartridge compartment cover 671 may detachably engage the main housing portion 621 via multiple tabs and interfacing tab openings (not illustrated) on the main housing portion 621.

As further illustrated in FIG. 50, at least one saline cartridge 664 and at least one medicament stock cartridge 666 may be insertable into the cartridge compartment 670. The fluid distribution manifold 660 may be configured such that, upon placement into the cartridge compartment 670, the saline cartridge 664 and the medicament stock cartridge 666 may be disposed in fluid communication with the fluid distribution manifold passage 661 in the fluid distribution manifold 660, as illustrated in FIGS. 48 and 49.

The saline cartridge 664 may contain a supply of normal saline. The medicament stock cartridge 666 may contain a supply of medicament stock solution. For example and without limitation, in embodiments in which the medicament injector 601 is to be used in botulinum toxin injections, the medicament stock solution may include stock botulinum toxin solution. The quantity or volume and concentration of saline in the saline cartridge 664 and of the medicament stock solution in the medicament stock cartridge 666 may vary depending on the desired quantity or volume and concentration of the medicament 658 which is to be injected from the medicament cartridge 632 into the patient 218 in each injection procedure or treatment session. In some embodiments, the quantities or volumes and concentrations of the saline and of the medicament stock solution may be selected to achieve the same quantities, volumes, injection dosage unit(s) and concentrations for the medicament 658 as those which were heretofore described with respect to the medicament 158 in the medicament cartridge 132 of the medicament injector 101. In some embodiments, the quantities or volumes and concentrations of the saline and of the medicament stock solution may be selected to produce medicament 158 having reconstituted botulinum toxin for injection into the patient 218.

In some embodiments, the fluid distribution manifold 660 may include at least one fluid distribution manifold pump 662 (FIGS. 48 and 49). The fluid distribution manifold pump 662 may be disposed in fluid communication with the fluid distribution manifold passage 661. The fluid distribution manifold pump 662 may be configured to pump the selected quantity or volume of the saline from the saline cartridge 664 and of the medicament stock solution from the medicament stock cartridge 666 through the fluid distribution manifold passage 661 into the main cartridge portion 633 of the medicament cartridge 632 for subsequent mixing of the saline solution and the medicament stock solution to form the medicament 658, as the cartridge plunger 656 deploys in the main cartridge portion 633. The user interface 604 of the medicament injector 601 may operably interface with the fluid distribution manifold pump 662 to facilitate programmed and/or real-time operation of the fluid distribution manifold pump 662. The user interface 604 may include the capability to facilitate user selection of the desired quantities or volumes of the saline solution and/or the medicament stock solution which the fluid distribution manifold pump 662 is to pump into the medicament cartridge 632 for subsequent mixing in formation of the medicament 658 during each injection.

As further illustrated in FIGS. 48 and 49, in some embodiments, the actuator piston 646 of the medicament injection assembly 644 may be configured to directly engage the cartridge plunger 656 in the main cartridge portion 633 of the medicament cartridge 632. Upon deployment from the retracted actuator piston position to the extended actuator piston position, the actuator piston 646 may deploy the cartridge plunger 656 in the main cartridge portion 633 of the medicament cartridge 632. The advancing cartridge plunger 656 may facilitate mixing of the saline solution and the stock medicament solution in the main cartridge portion 633 to form the medicament 658 before or as the medicament 658 is discharged from the main cartridge portion 633 through the injection nozzle 634 and the injection opening 638, respectively, in the cartridge leveling disk 637. In alternative embodiments, the medicament injection assembly 644 may include a plunger piston (not illustrated) such as the plunger piston 149 of the medicament injector 101 which was heretofore described with respect to FIGS. 5-13. Accordingly, the actuator piston 646 of the medicament injection assembly 644 may directly engage the plunger piston 149. The plunger piston 149 may disengage the cartridge plunger 656 of the medicament cartridge 632 in the safety position and engage the cartridge plunger 656 in the primed position, as was heretofore described with respect to the medicament injector 101.

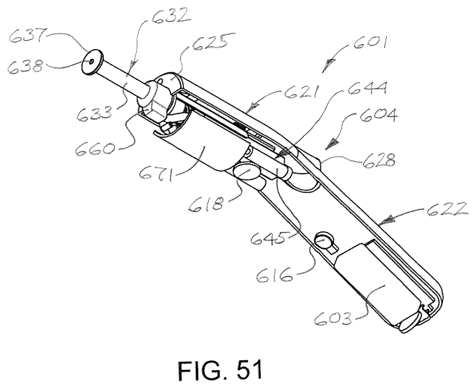

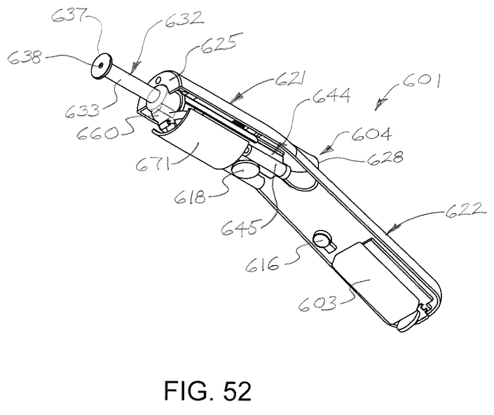

In some embodiments, the medicament injector 601 may include the safety mechanism 651. The fluid distribution manifold 660 and the safety spring housing 652 may be selectively deployable in the safety position illustrated in FIGS. 48 and 51 and in the primed position illustrated in FIGS. 49 and 52 for use of the medicament injector 601. As illustrated in FIGS. 48 and 51, in the safety position, the fluid distribution manifold 660 may protrude beyond the front housing surface 625 of the main housing portion 621, exterior to the main housing portion 621, with the safety spring housing 652 inside the main housing portion 621 such that the actuator piston 646 disengages the cartridge plunger 656 and a gap (not numbered) exists therebetween. In the primed position, illustrated in FIGS. 49 and 52, the fluid distribution manifold 660 may be retracted into the main housing portion 621 such that the gap between the actuator piston 646 and the cartridge plunger 656 is closed and the actuator piston 646 engages the cartridge plunger 656.

In some embodiments, the medicament injection assembly 644 may include an electromagnetic actuator 645. The electromagnetic actuator 645 may be as was heretofore described with respect to the electromagnetic actuator 145 of the medicament injector 101. In other embodiments, the medicament injection assembly 644 may include a gas actuator such as the gas actuator 345 of the medicament injector 301 which was heretofore described with respect to FIGS. 23-29 or the gas actuator 445 of the medicament injector 401 which was heretofore described with respect to FIGS. 30-36. In some embodiments, the medicament injection assembly 644 may include a spring actuator such as the spring actuator 545 of the medicament injection assembly 501 which was heretofore described with respect to FIGS. 37-43.

Application of the medicament injector 601 may be as was heretofore described with respect to the medicament injector 101 in FIGS. 1-22. Preparatory to an injection procedure, the saline cartridge 664 and the medicament stock cartridge 666 may be deployed in place in the cartridge compartment 670 in the main housing portion 621 of the system housing 620. The cartridge compartment cover 671 may be closed on the cartridge compartment 670. The medicament cartridge 632 may be deployed from the safety position illustrated in FIGS. 48 and 51 to the primed position illustrated in FIGS. 49 and 52.

After placement of the medicament injector 601 against the skin 220 on the face 219 of the patient 218 preparatory to injecting the medicament 658 into or beneath the skin 220 for each injection procedure in a treatment session, the trigger 628 and/or other control feature(s) of the user interface 604 may be actuated to facilitate actuation of the medicament injection assembly 644. The fluid distribution pump 662 may first be actuated to pump the saline solution from the saline cartridge 664 and the stock medicament solution from the medicament stock cartridge 666 through the fluid distribution passage 661 into the main cartridge portion 663 of the medicament cartridge 662. The electromagnetic actuator 645 may then deploy the actuator piston 646 from the retracted actuator piston position illustrated in FIGS. 49 and 51 to the extended plunger piston position (not illustrated). The advancing actuator piston 646 may advance the cartridge plunger 656 in the main cartridge portion 633 of the medicament cartridge 632 such that the cartridge plunger 656 mixes the saline solution and the stock medicament solution to form the medicament 658 in the main cartridge portion 633 of the medicament cartridge 632 and then discharges the medicament 658 from the main cartridge portion 633 through the injection nozzle 634 and the injection opening 638, respectively, in the cartridge leveling disk 637 into or beneath the skin 220 of the patient 218 in the selected quantity, volume and/or number of injection dosage units for the injection procedure. In some embodiments and applications, mixture of the saline solution and the stock medicament solution in the medicament cartridge 632 during deployment of the cartridge plunger 656 may facilitate reconstitution of botulinum toxin for injection as the medicament 658. As deemed necessary during the treatment session, a replacement saline cartridge 664 and/or medicament stock cartridge 666 may be deployed in place in the cartridge compartment 670 of the injector housing 620.

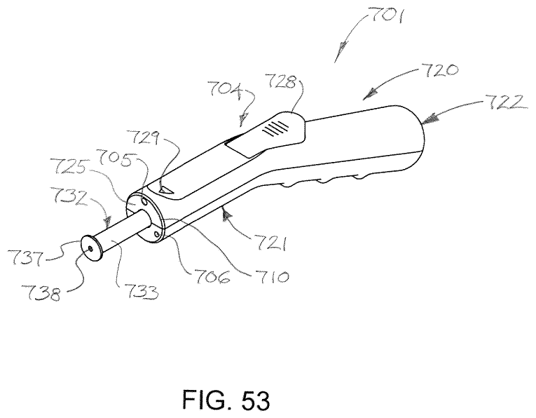

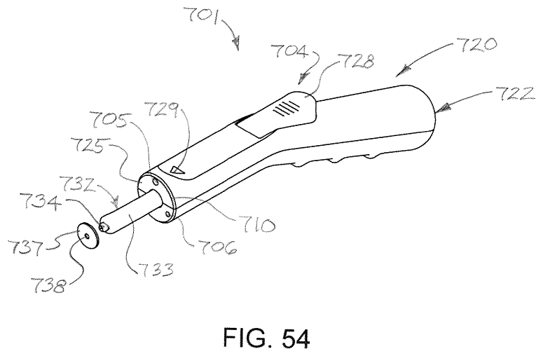

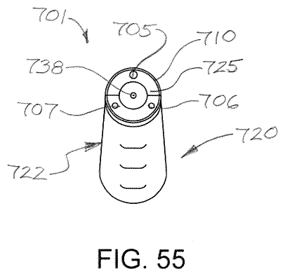

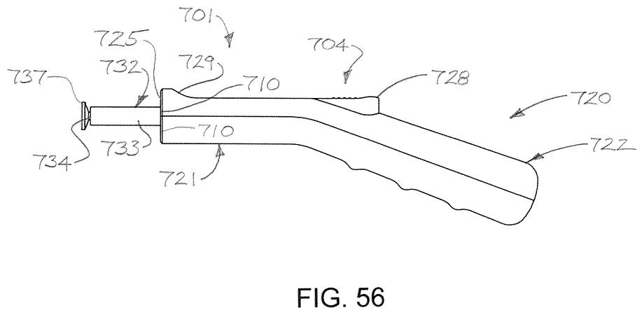

### Alternative Medicament Injector—FIGS. 53-60

Referring next to FIGS. 53-60 of the drawings, an illustrative disposable embodiment of the medicament injector of the medicament injection systems is generally indicated by reference numeral 701. The medicament injector 701 may be suitable for use with the medicament injection system 100 which was heretofore described with respect to FIGS. 1-22. Unless otherwise indicated, elements of the medicament injector 701 which are structurally and/or functionally analogous to the respective elements of the medicament injector 101 that was heretofore described with respect to FIGS. 1-22 are designated by the same respective reference numerals in the 701-799 series in FIGS. 53-60. Accordingly, to the extent which is applicable, the same description which was heretofore described with respect to the medicament injector 101 is incorporated by reference herein in its entirety herein with respect to the medicament injector 701.

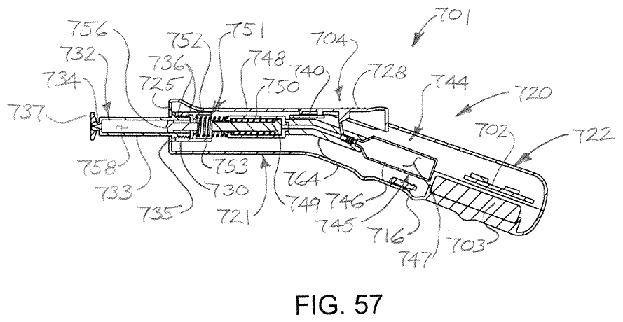

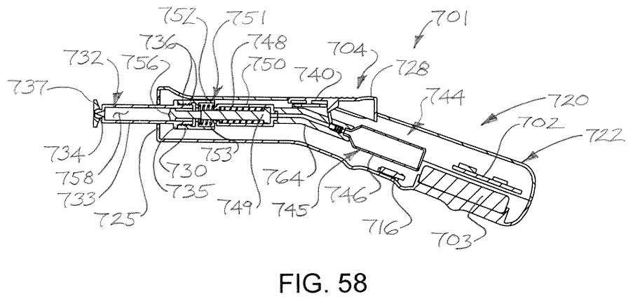

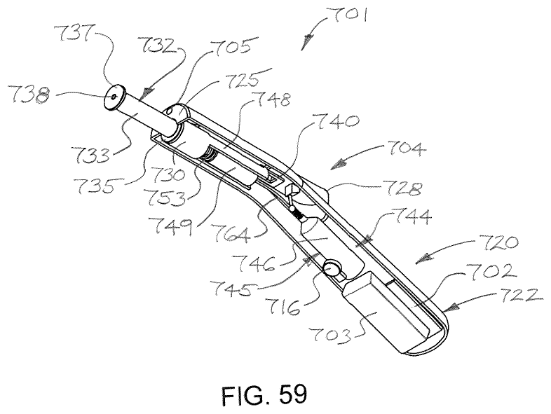

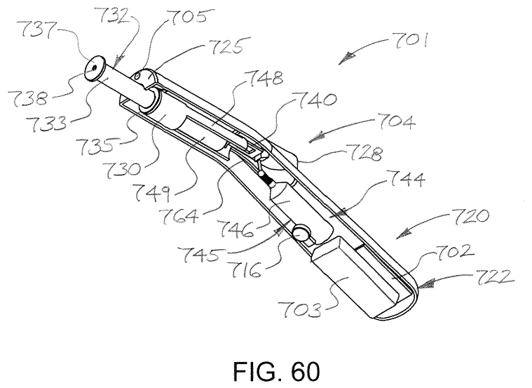

The medicament injector 701 may be used disposably and may be configured for single-use applications. Accordingly, the various components of the medicament injector 701 may be fabricated of biodegradable or recyclable plastics and/or other materials. As illustrated in FIGS. 57 and 58, in some embodiments, the medicament cartridge 732 may be un-removably or un-detachably mounted with respect to the main housing portion 721 of the injector housing 720. The main cartridge portion 733 of the medicament cartridge 732 may be slidably mounted within a cartridge opening (not illustrated) in the front housing surface 725. The medicament cartridge 732 may thus be selectively deployable from the safety position illustrated in FIGS. 57 and 59 to the primed position illustrated in FIGS. 58 and 60, against the bias imparted by the safety spring 753 of the safety mechanism 751, preparatory to use of the medicament injector 701.

As illustrated in FIGS. 57-60, in some embodiments, the medicament injection assembly 744 of the medicament injector 701 may include a gas actuator 745. The gas actuator 745 may be the same as or similar to the gas actuator 345 of the medicament injector 301 which was heretofore described with respect to FIGS. 23-29 or the gas actuator 445 of the medicament injector 401 which was heretofore described with respect to FIGS. 30-36. The gas actuator 745 may include at least one gas cartridge 746. The gas cartridge 746 may be configured to contain a supply of pressurized gas 747. The pressurized gas 747 may include compressed air, carbon dioxide and/or other compressed gas or combination of compressed gases suitable for the purpose of the gas actuator 745 as described herein. The trigger 728 and/or other control feature(s) of the user interface 704 may operably interface with the gas cartridge 746, to facilitate selective release of the pressurized gas 747 from the gas cartridge 746.

A plunger piston housing 748 may be provided in the injector housing 720, forwardly of the gas cartridge 746. A plunger piston 749 may be slidably disposed between a retracted plunger piston position (illustrated in FIGS. 57 and 58) and an extended plunger piston position (not illustrated) in the plunger piston housing 748. The gas cartridge 746 of the gas actuator 745 may be disposed in fluid communication with the plunger piston housing 748 such as via a gas passage 764. As it flows from the gas cartridge 746 into the plunger piston housing 748, the pressurized gas 747 may apply forward pressure against the plunger piston 749 such that the plunger piston 749 is deployed from the retracted plunger piston position toward the extended plunger piston position, against the cartridge plunger 756 of the medicament cartridge 732. Continued extension of the plunger piston 749 to the extended plunger piston position may cause the plunger piston 749 to advance the cartridge plunger 756 forwardly in the main cartridge portion 733 of the medicament cartridge 732 such that the cartridge plunger 756 expels at least a predetermined quantity, volume, or number of injection dosage units of the medicament 758 from the main cartridge portion 733 through the injection nozzle 734 and the injection opening 738 in the cartridge leveling disk 737, respectively.

In some embodiments, the medicament injection assembly 744 may include the gas actuator 745, as was heretofore described. In other embodiments, the medicament injection assembly 744 may include an electromagnetic actuator such as the electromagnetic actuator 145 of the medicament injector 101 which was heretofore described with respect to FIGS. 5-13. In some embodiments, the medicament injection assembly 744 may include a spring actuator such as the spring actuator 545 of the medicament injection assembly 501 which was heretofore described with respect to FIGS. 37-43.

Application of the medicament injector 701 may be as was heretofore described with respect to application of the medicament injector 101. After use, the medicament injector 101 may be discarded. A replacement medicament injector 701 may subsequently be used for additional injection procedures and/or treatment sessions.

### Example Medicament Dosing and Loading Device

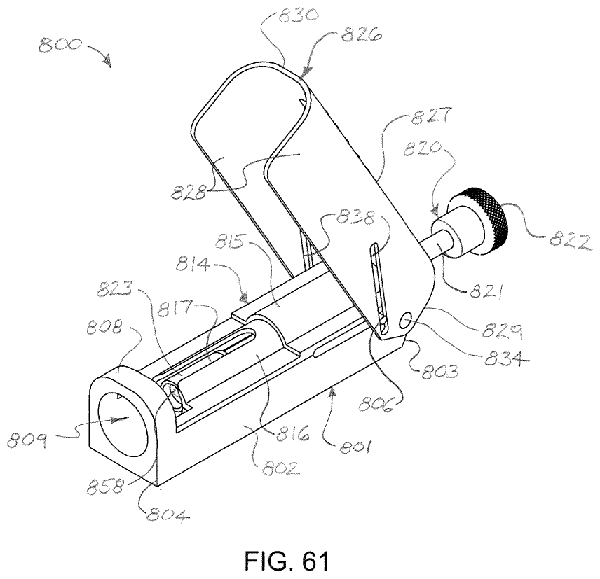

Referring next to FIGS. 61-68 of the drawings, an illustrative embodiment of a medicament dosing and loading device which is suitable for loading medicament 858 (FIG. 61) into a medicament cartridge 832 of a medicament injector 851 is generally indicated by reference numeral 800. In various applications, the medicament injector 851 the medicament cartridge 832 of which is to be dosed and loaded with medicament 858 using the medicament dosing and loading device 800 may include but is not limited to the medicament injector 101 (FIGS. 5-13); the medicament injector 301 (FIGS. 23-29); the medicament injector 401 (FIGS. 30-36); the medicament injector 501 (FIGS. 37-43); the medicament injector 601 (FIGS. 44-52); or the medicament injector 701 (FIGS. 53-60). The medicament dosing and loading device 800 may be used to dose and load the medicament 858 into the medicament cartridge 832 for a one-time use of the medicament cartridge 832 or for reloading of the medicament cartridge 832 for multiple uses.

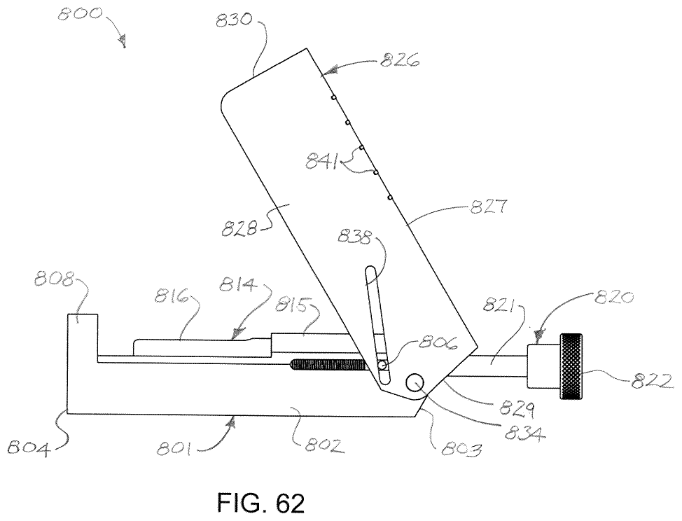

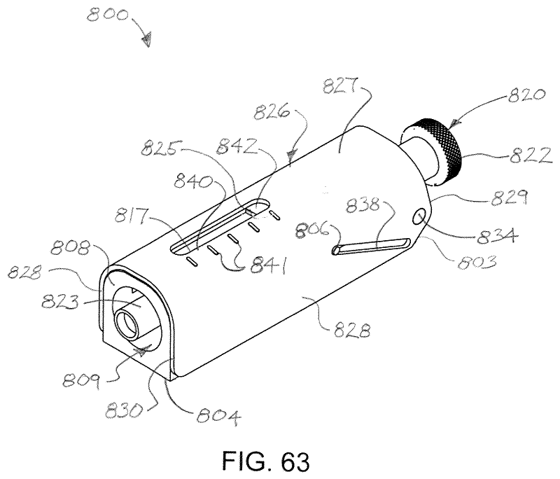

The medicament dosing and loading device 800 may include a device base 801. A device cover 826 may be pivotally attached to the device base 801. The device cover 826 may be selectively deployable in an open cover position, illustrated in FIGS. 61 and 62, and a closed cover position, illustrated in FIG. 63, for purposes which will be hereinafter described.

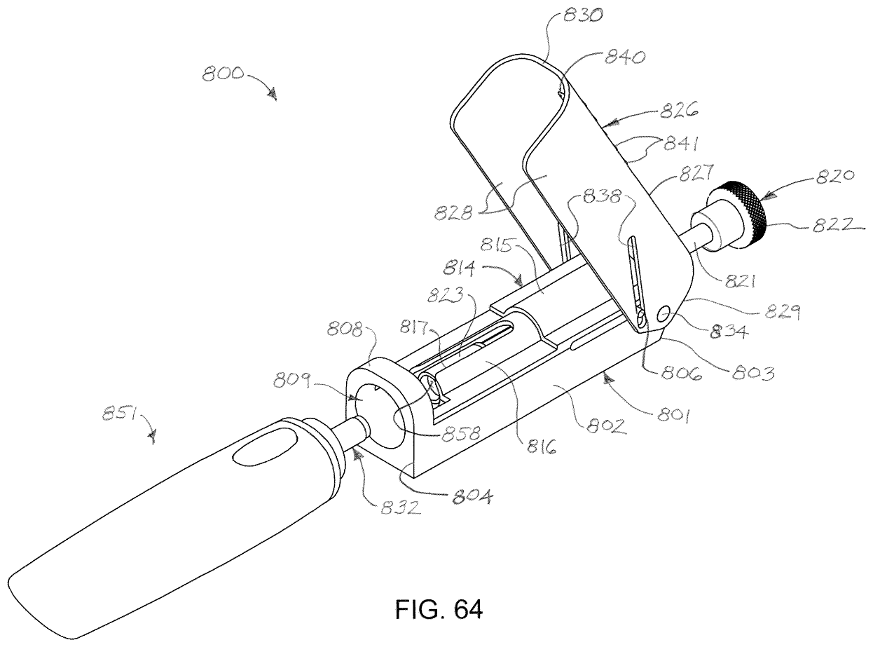

The device base 801 may include an elongated main base portion 802 having a first base end 803 and a second base end 804. As illustrated in FIGS. 61 and 64, the device base 801 may be configured to receive and accommodate a replaceable medicament loading container 823 which contains a supply of the medicament 858 to be loaded from the medicament loading container 823 into the medicament cartridge 832 of the medicament injector 851. In some embodiments, the medicament loading container 823 may be transparent or translucent. A loading plunger assembly 820 may be disposed in the device base 801. The loading plunger assembly 820 may protrude from the first base end 803 of the main base portion 802. The loading plunger assembly 820 may be configured to load the desired quantity, volume, and/or number of injection dosage units of the medicament 858 from the medicament loading container 823 into the medicament cartridge 832, as will be hereinafter described.

A pair of cover guide pins 806 may extend outwardly from the respective sides of the main base portion 802. A pair of pin slots 838 may be provided in the device cover 826. The cover guide pins 806 may slidably engage the respective pin slots 838 to guide the device cover 826 as the device cover 826 deploys between the open and closed cover positions.

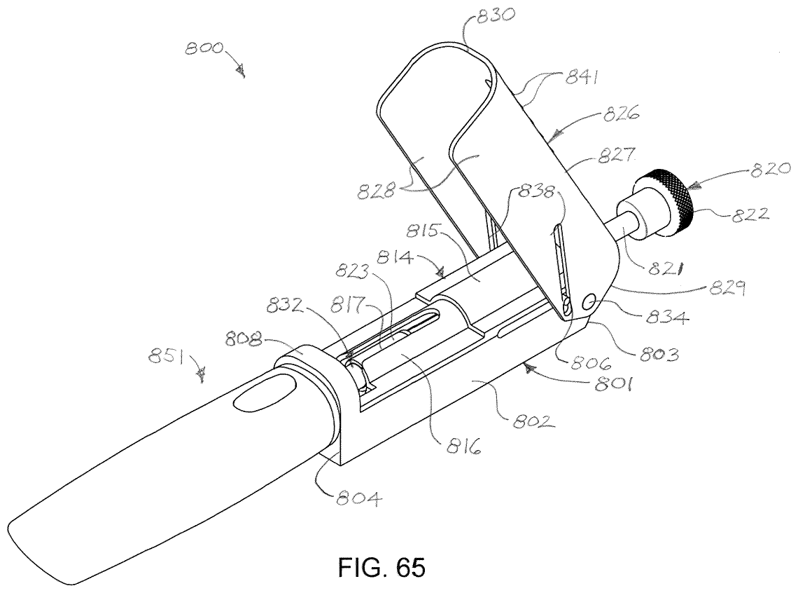

A base flange 808 may be provided at the second base end 804 of the main base portion 802 of the device base 801. An injector opening 809 may extend through the base flange 808. As illustrated in FIGS. 64 and 65, the injector opening 809 may be suitably sized and configured to receive and accommodate the front-end portion of the medicament injector 851 in dosing and loading of the medicament 858 into the medicament cartridge 832 on the medicament injector 851.

As illustrated in FIG. 61, the device base 801 may further include a base cover 814 on the main base portion 802. The base cover 814 may include a main cover portion 815. A loading chamber 816 may extend from the main cover portion 815 toward the base flange 808. The loading chamber 816 may be suitably sized and configured to receive and accommodate the medicament loading container 823. An elongated cover slot 817 may be provided in the loading chamber 816 of the base cover 814. The cover slot 817 may overlie the medicament loading container 823 to render visible the medicament 858 in the medicament loading container 823 therethrough.

The device cover 826 may have an elongated main cover portion 827. A pair of parallel, spaced-apart side cover portions 828 may extend from the main cover portion 827. The main cover portion 827 and the side cover portions 828 of the device cover 826 may have a first cover end 829 and a second cover end 830. The first cover end 829 of the device cover 826 may be pivotally attached to the first base end 803 of the main base portion 802 of the device base 801 via a cover pivot 834.

As illustrated in FIG. 63, an elongated medicament indicator slot 840 may be provided in the main cover portion 827 of the device cover 826. The medicament indicator slot 840 may coincide in position and length with the cover slot 817 in the loading chamber 816 of the base cover 814 when the device cover 826 is in the closed cover position on the device base 801. Graduated volume markings 841 may be provided on the main cover portion 827 along the length of the cover slot 817. The graduated volume markings 841 may correspond to the quantity, volume, and/or number of injection dosage units of the medicament 858 in the medicament loading container 823.

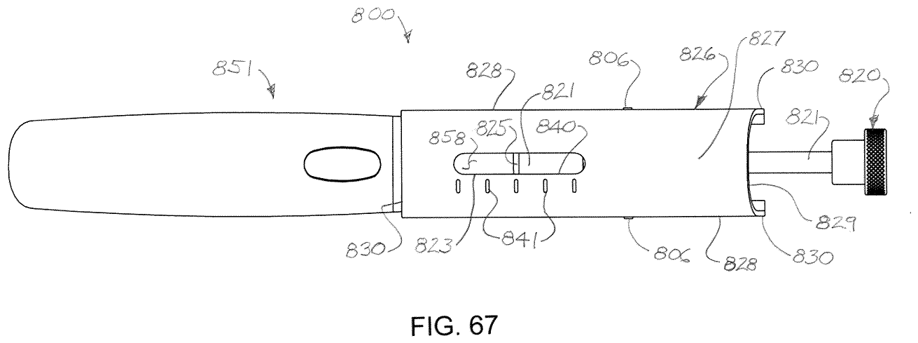

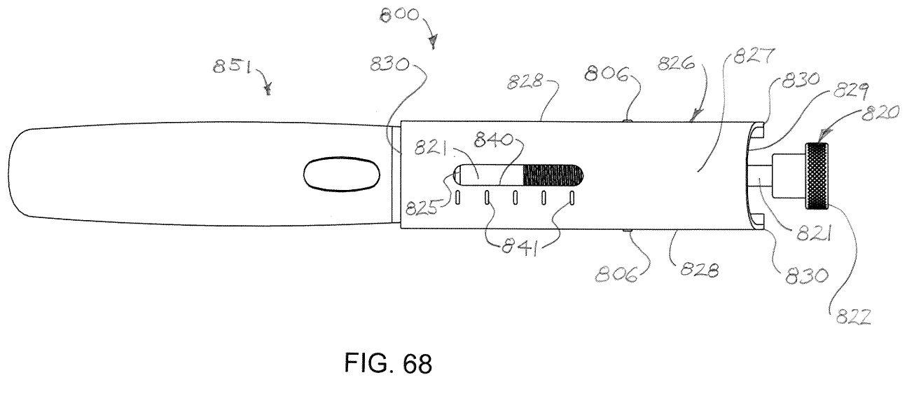

The loading plunger assembly 820 may include an elongated plunger shaft 821. The plunger shaft 821 of the loading plunger assembly 820 may be slidably mounted in a plunger shaft opening (not illustrated) in the device base 801. Accordingly, the plunger shaft 821 may be selectively deployable between an extended, pre-loading position, as illustrated in FIG. 67, and a retracted, loading position illustrated in FIG. 68. A plunger knob 822 may be provided on the end of the plunger shaft 821 which protrudes beyond the first base end 803 of the main base portion 802. The opposite end of the plunger shaft 821 may terminate in the loading chamber 816 of the base cover 814 at the upstream end, or opposite the discharge end, of the medicament loading container 823. A medicament volume indicator 825 may be slidably disposed in the medicament loading container 823. As it is deployed from the pre-loading position to the loading position for loading of the medicament 858 into the medicament cartridge 832, the plunger shaft 821 of the loading plunger assembly 820 may engage and push the medicament volume indicator 825 in and along the medicament loading container 823 to expel the medicament 858 from the medicament loading container 823 into the medicament cartridge 832. The medicament volume indicator 825 may simultaneously sequentially align or register with the respective volume markings 841 on the device cover 826 to indicate the quantity, volume, and/or number of injection dosage units of the medicament 858 which is loaded into the medicament cartridge 832.

In some example applications, the medicament dosing and loading device 800 may be used to fill or refill the medicament cartridge 832 in the medicament injector 851 with a selected quantity, volume, and/or number of injection dosage units of the medicament 858 for subsequent use of the medicament injector 851 with the system 100 in a treatment session. Accordingly, the plunger shaft 821 of the loading plunger assembly 820 may initially be deployed in the extended, pre-loading position in the device base 801, as illustrated in FIGS. 64 and 65. A medicament loading container 823 which contains a supply of the medicament 858 may be deployed in place in the loading chamber 816 of the base cover 814 on the device base 801. In some applications, this may be accomplished by inserting the medicament loading container 823 into the loading chamber 816 through the injection opening 809 in the base flange 808. Alternatively, the device cover 826 may be opened on the device base 801 to facilitate placement of the medicament loading container 823 in the loading chamber 816.

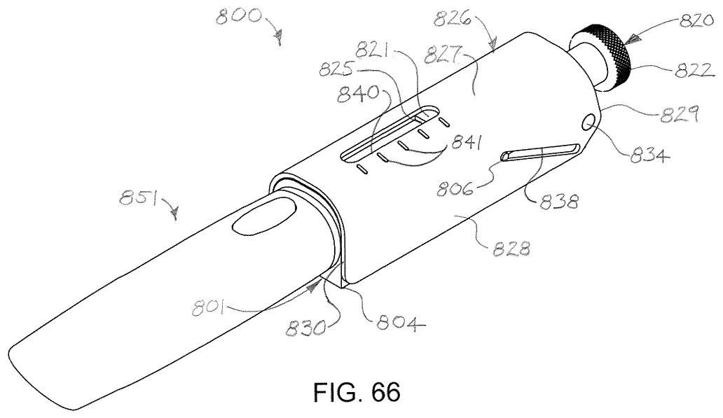

As illustrated in FIG. 64, the medicament injector 851, having the medicament cartridge 832 which is to be filled or refilled with the medicament 658, may be inserted through the injection opening 809 in the base flange 808 until the medicament cartridge 832 engages or inserts into the downstream or discharge end of the medicament loading container 823. As illustrated in FIG. 65, the device cover 826 may remain open to verify the correct positioning of the medicament cartridge 832 with respect to the medicament loading container 823. The device cover 826 may then be closed, as illustrated in FIG. 66.

As illustrated in FIG. 67, the plunger shaft 821 of the loading plunger assembly 820 may then be slowly deployed from the extended, pre-loading position to the retracted, loading position in the device base 801. Accordingly, the medicament volume indicator 825 on the extending or distal end of the plunger shaft 821 may engage and push the medicament volume indicator 825 in and along the medicament loading container 823 to expel the medicament 858 from the medicament loading container 823 into the medicament cartridge 832. As it deploys, the medicament volume indicator 825 may sequentially align or register with the respective volume markings 841 on the device cover 826 to indicate the quantity, volume, and/or number of injection dosage units of the medicament 858 which is loaded into the medicament cartridge 832.

After the selected quantity, volume, and/or number of injection dosage units of the medicament 658 has been discharged from the medicament loading container 823 into the medicament cartridge 832, the medicament injector 851 may be removed from the injection opening 809. The medicament injector 851 may then be used with the medicament injection system 100 as was heretofore described. Prior to subsequent filling or refilling of a medicament cartridge 832 with medicament 858, a replacement medicament loading container 823 may be placed in the loading chamber 816 of the device base 801, as was heretofore described.

### Example Medicament Injection Methods

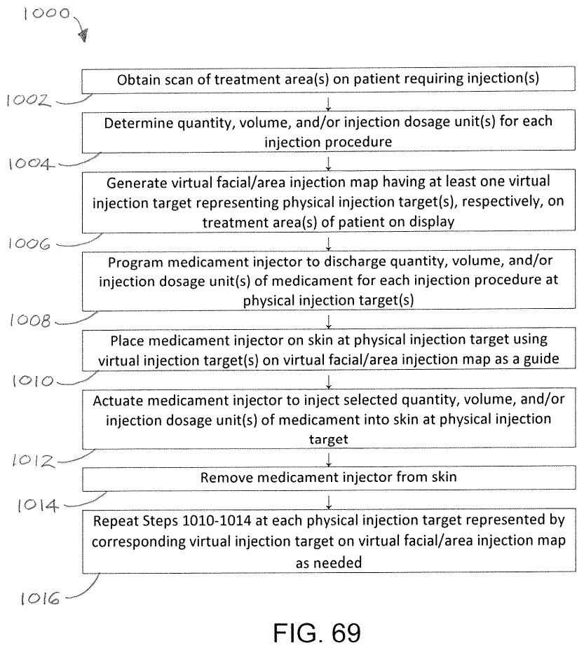

Referring next to FIG. 69 of the drawings, a flow diagram of an illustrative embodiment of the medicament injection methods is generally indicated by reference numeral 1000. At Step 1002 of the method 1000, a 3-dimensional scan of at least one treatment area which requires at least one injection of medicament on a patient may be obtained.

At Step 1004, the quantity, volume, and/or number of injection dosage units for each injection procedure at the scanned treatment area may be determined.

At Step 1006, a 3-dimensional virtual facial/area injection map may be generated on a display using the 3-dimensional scan. The virtual facial/area injection map may have at least one virtual injection target which represents the corresponding location or position of at least one physical injection target on the treatment area of the patient.

At Step 1008, a medicament injector may be programmed to discharge the quantity, volume, and/or number of injection dosage units of the medicament determined at Step 1004 for each injection procedure at each corresponding physical injection target.

At Step 1010, using the virtual injection target(s) on the virtual facial/area injection map as a guide, the medicament injector may be placed on or against the skin of the patient at a physical injection target which corresponds to one of the virtual injection target(s).

At Step 1012, the medicament injector may be actuated to inject the selected quantity, volume, and/or number of injection dosage units of the medicament into or beneath the skin at the physical injection target.

At Step 1014, the medicament injector may be removed from the skin.

At Step 1016, Steps 1010-1014 may be repeated at each physical injection target on the treatment area the location or position of which is represented by a corresponding virtual injection target on the virtual facial/area injection map, as needed.

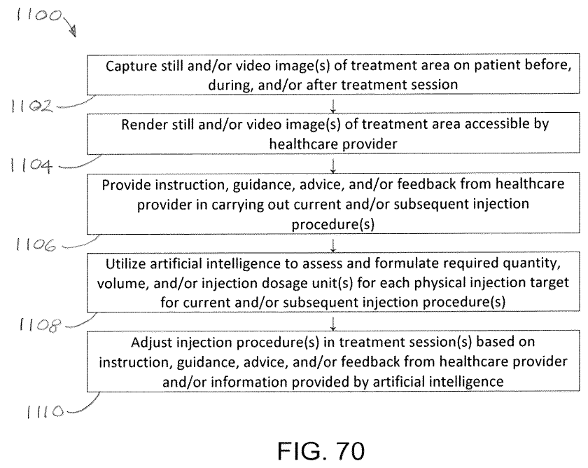

Referring next to FIG. 70 of the drawings, a flow diagram which illustrates an example of utilization of instruction, guidance, advice, and/or feedback from a remote healthcare provider and/or information from artificial intelligence in carrying out current and/or subsequent injection procedures according to some embodiments of the medicament injection methods is generally indicated by reference numeral 1100. At Step 1102 of the method 1100, at least one still and/or video image of the treatment area on the patient before, during, and/or after a treatment session may be obtained.

At Step 1104, the still and/or video image(s) of the treatment area obtained at Step 2002 may be rendered accessible by a remote healthcare provider.

At Step 1106, instruction, guidance, advice, and/or feedback from the remote healthcare provider may be provided in carrying out current and/or subsequent injection procedure(s) in a treatment session.

At Step 1108, artificial intelligence may be used to assess and formulate the required quantity, volume, and/or number of injection dosage units for each physical injection target for current and/or subsequent injection procedure(s).

At Step 1110, the injection procedure(s) in a treatment session may be adjusted based on the instruction, guidance, advice, and/or feedback from the remote healthcare provider obtained at Step 1106 and/or based on the information provided by the artificial intelligence at Step 1108. Adjustments in the injection procedures(s) may include adjustments in such parameters as the quantity, volume, and/or number of injection dosage units of the medicament which is to be injected into or beneath the skin at each physical injection target in the treatment area during a treatment session; the frequency and scheduling of treatment sessions; and the like.

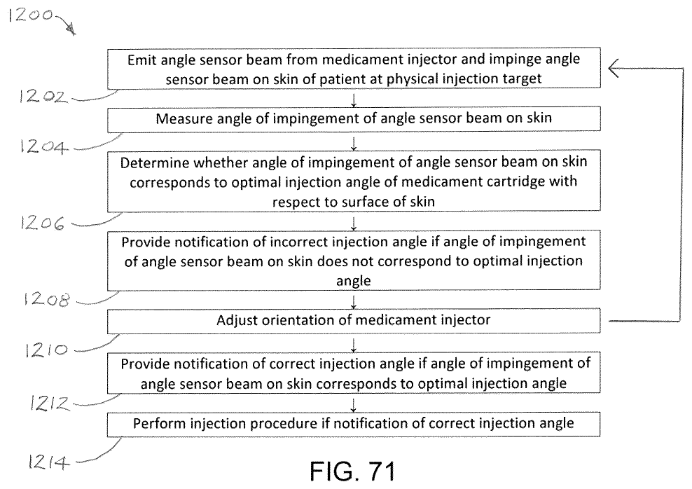

Referring next to FIG. 71 of the drawings, a flow diagram which illustrates an example of determination of whether an injection angle of the medicament injector with respect to the skin of the patient is optimal for implementation of an injection procedure at a physical injection target on the skin of a patient in a treatment area is generally indicated by reference numeral 1200. At Step 1202 of the method 1200, an angle sensor beam may be emitted from the medicament injector. The angle sensor beam may impinge on the skin of the patient at the physical injection target.

At Step 1204, an angle of impingement of the angle sensor beam on the skin of the patient may be measured.

At step 1206, a determination may be made as to whether the angle of impingement of the angle sensor beam on the patient's skin, obtained at Step 1204, corresponds to an optimal injection angle of a medicament cartridge on the medicament injector with respect to the surface of the skin.

At Step 1208, notification of an incorrect injection angle may be provided if the angle of impingement of the angle sensor beam on the patient's skin does not correspond to the optimal injection angle.

At Step 1210, if notification of an incorrect injection angle is provided at Step 1208, then the orientation of the medicament injector may be adjusted. Steps 1202-1208 may subsequently again be carried out.

At Step 1212, notification of a correct injection angle may be provided if the angle of impingement of the angle sensor beam on the patient's skin corresponds to the optimal injection angle.

At Step 1214, if the correct injection angle is indicated at Step 1212, then the injection procedure may be performed.

It will be recognized and understood by those skilled in the art that the method 1000 heretofore described with respect to FIG. 69, the method 1100 heretofore described with respect to FIG. 70, and the method 1200 heretofore described with respect to FIG. 71 may each be carried out using the system 100 which was heretofore described with respect to FIGS. 1-22 as well as any of the medicament injectors 101, 301, 401, 501, 601, and 701, respectively, and the medicament dosing and loading device 800. Accordingly, the preceding descriptions of the system 100; the medicament injectors 101, 301, 401, 501, 601, and 701, respectively; and the medicament dosing and loading device 800 are hereby incorporated by reference herein in their entireties with respect to the methods 1000, 1100, and 1200. The methods 1000, 1100 and 1200 may be carried out via self-administration by a patient or by and attending healthcare provider or untrained assisting person. Each of the methods 1000, 1100, and 1200 may be carried out using additional and/or alternative steps described herein, and the steps may be carried out in different orders in some embodiments. Methods in addition or alternative to the methods 1000, 1100, and 1200 are contemplated by the descriptions embodied in the present disclosure and the appended claims.

### Example Custom Medicament Injection System

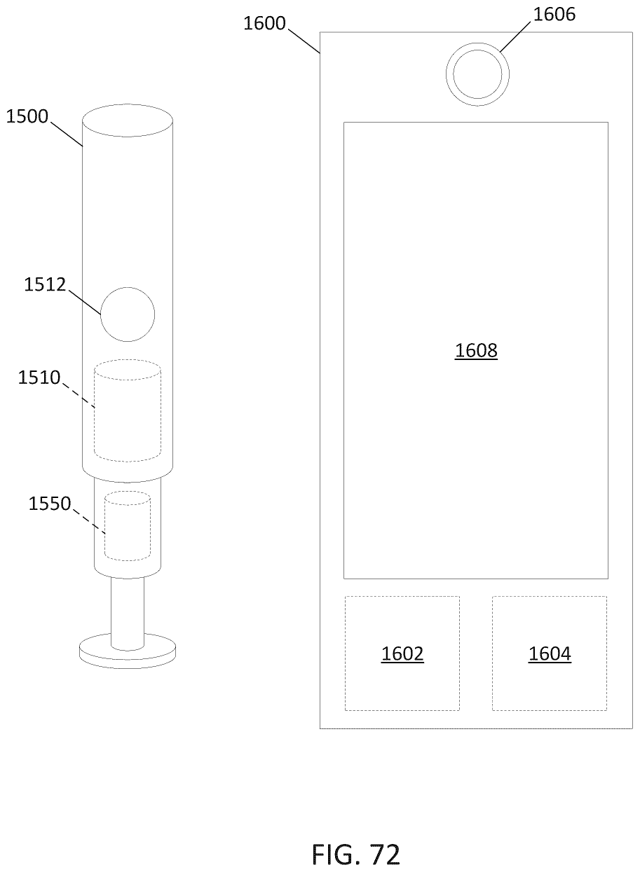

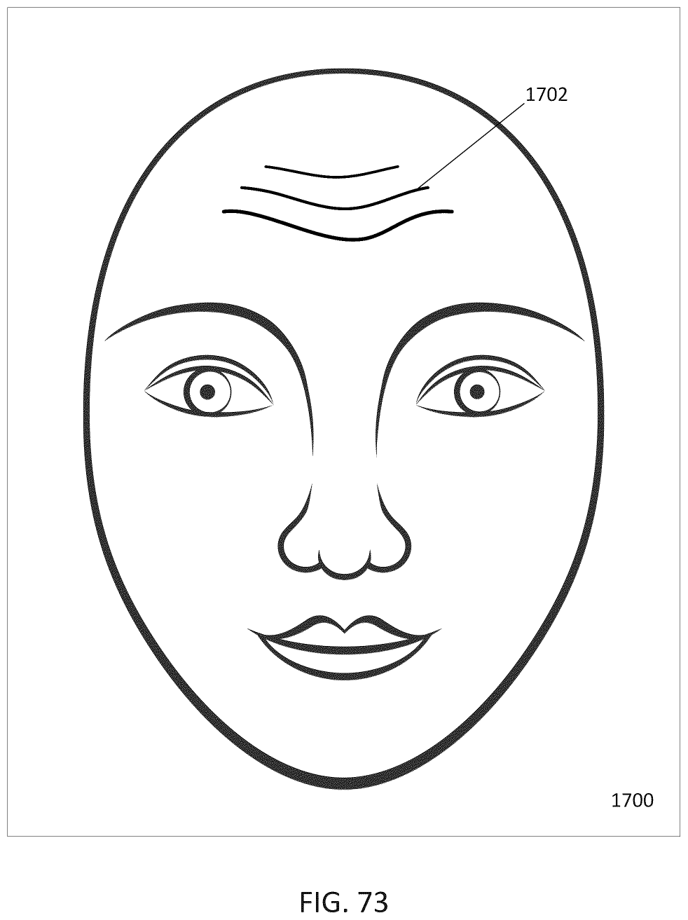

FIG. 72 shows an example of a custom medicament injection system including a medicament injection component 1500 and a computing device 1600.

The medicament injection component 1500 schematically illustrated in FIG. 72 may be the same as or similar to, or include the same or similar components or functionality as, the medicament injectors described above in other examples. In the example of FIG. 72 the medicament injection component 1500 includes an injector 1510 and a replaceable medicament cartridge 1550. The injector 1510 is configured such that when it is positioned against a person's skin and actuated (i.e. via actuator 1512) the injector 1510 injects a set medicament dosage from the replaceable medicament cartridge 1550 into or beneath the person's skin.

The computing device 1600 schematically illustrated in FIG. 72 may be the same as or similar to, or include the same or similar components or functionality as, the treatment management subsystems described above in other examples. In the example of FIG. 72 the computing device 1600 includes a processor device 1602 and a non-transitory computer-readable medium 1604. The computer-readable medium 1604 has instructions stored on it that are executable by the processor device 1602 to receive pre-treatment data about a treatment area associated with a person's skin, and then, based on the received pre-treatment data, identify one or more target medicament injection sites relative to the treatment area, and then output targeting data for the one or more target medicament injection sites relative to the treatment area.

In some implementations the captured pre-treatment data may be or include at least one captured image of the treatment area captured using a camera 1606 of the computing device 1600. The captured image may be a static image or a video image of the treatment area including at least one indicia associated with a muscle animation to be treated by the custom medicament injection system. The computer readable medium 1604 of the computing device 1600 has instructions executable by the processor device 1602 to cause the computing device 1600 to identify target medicament injection sites relative to the treatment area based on a spatial relationship of the indicia with the animated muscle.

The display 1608 of the computing device may be used to output targeting data. In some implementations, the display 1608 may output targeting data by showing target medicament injections sites overlaid on an image including the treatment area. The displayed image may be a live image captured and displayed by the computing device 1600 in real time or may be the previously captured image or another image.

The image displayed on display 1608 provides guidance to a user for aligning the medicament injection component 1500 with the target medicament sites. In some implementations the guidance is simply the image itself, showing the user where the target medicament sites are relative to the treatment area. In other implementations the medicament injection component 1500 and/or the computing device 1600 may include additional components and/or functionality allowing the system to track the relative spatial position and orientation of the medicament injection component 1500 relative to the treatment area and provide feedback (e.g. visual, audible, haptic, etc.) to the user to confirm proper alignment of the medicament injection component 1500 relative to the target medicament sites prior to injection.

As discussed above the computing device 1600 is configured to receive pre-treatment data about a treatment area associated with the person's skin and, based on that data, identify one or more target medicament injection sites relative to the treatment area. In one implementation the computing device 1600 utilizes machine vision or computer vision technologies to process the captured image and identify indicia in the image associated with a muscle animation to be treated by the system. The computing device 1600 may use a trained algorithmic model or neural network or other image processing technique to identify the type of indicia in the captured image. As a few examples, the computing device 1600 may identify one or more of the following indicia types in the captured image: (1) horizontal forehead lines (associated with animation of the frontalis), (2) frown lines (associated with animation of the glabellar complex), (3) crow's feet (associated with animation of the orbicularis oculi), (4) bunny lines (associated with animation of the nasalis), (5) nasolabial folds (associated with animation of the levator labii superioris alaequae nasi), (6) radial lip lines (associated with animation of the orbicularis oris), (7) marionette lines (associated with animation of the depressor anguli oris), (8) chin lines (associated with animation of the mentalis), and/or (9) other rhytids in the captured image. In addition to identifying the type of indicia, the computing device 1600 may also determine other aspects of the indicia, including its relative size, position, or other aspects associated with the indicia, or other aspects of the treatment area (e.g. relationship between the identified indicia and other features of the treatment area, such as the eyebrows, nose, eyes, and/or lips of the treatment area).

Based on the computing device's identification of indicia in the captured image (e.g. type and location of the indicia in the image) the computing device 1600 may generate, determine, or obtain target medicament injection sites relative to the target area. In one example, the computing device 1600 may access a database, table, or other relational data set that links a specific target injection site pattern to the identified indica type, with the specific target injection site pattern being based on a spatial relationship between the identified indicia and an animated muscle associated with the indicia. For example, the computing device 1600 may access a database (either stored on the non-transitory computer readable medium 1604 or accessed remotely over a network) that includes data for one or more specific target injection site patterns appropriate for particular indicia. In other words, the database may include data for one or more specific target injection sites patterns appropriate for horizontal forehead lines (associated with animation of the frontalis), may include data for other specific target injection site patterns appropriate for frown lines (associated with animation of the glabellar complex), and may include data for other specific target injection site patterns appropriate for other indica that the system is configured for. This data may be used for outputting targeting data for target medicament injection sites relative to the treatment area.

In some implementations the database may include several target injection site patterns for each indicia type. For example, for indicia in the form of horizontal forehead lines, the database may include several target injection site patterns that may be selected from depending on additional characteristics of the horizontal forehead lines. For one simple example, the database may include one target injection site pattern for indicia with 3 or less horizontal forehead lines and a different target injection site pattern for indicia with 4 or more horizontal forehead lines. In another simple example, the databased may include one target injection site pattern for indicia in which the horizontal forehead lines cover relatively less area of the forehead, and a different target injection site pattern for indicia in which the horizontal forehead lines cover relative more area of the forehead. The different patterns may take the form or more or less injection sites, different patterns of injection sites, and/or different injection dosages.

In still other implementations the system does not utilize a pre-defined database or other pre-defined relational data set, but instead uses a trained neural network or other artificial intelligence functionality to determine targeting data based on the captured image. For example, a neural network may be trained using a large data set that includes numerous images of persons' faces with indicia indicating muscle animations along with appropriate targeting data with injection sites for treating the muscle animations. Once trained, the neural network may be configured to accept as an input an image or other pre-treatment data about a treatment area associated with a person's skin, and to output targeting data with one or more target medicament injection sites relative to the treatment area.

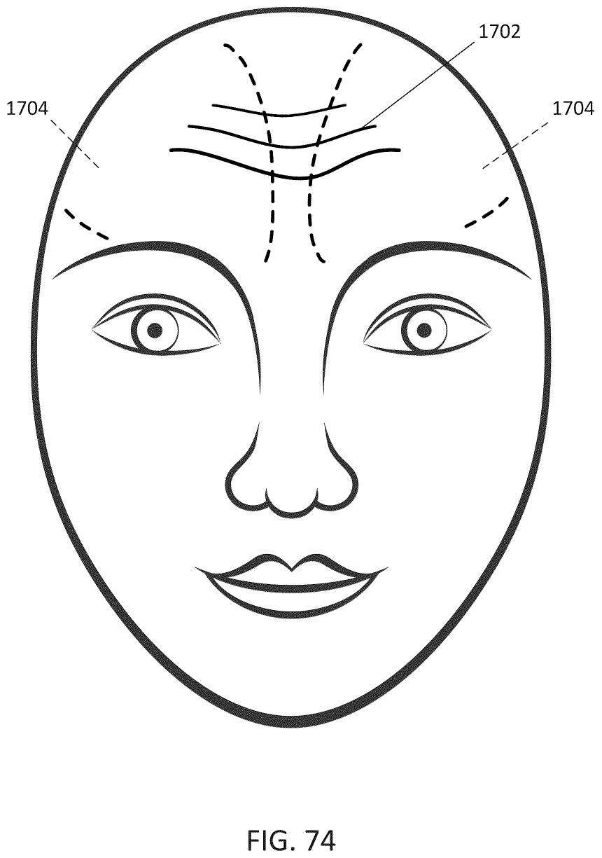

FIGS. 73-77 show one example of using a captured image including a treatment area to identify and output targeting data for target medicament injection sites relative to the treatment area. FIG. 73 shows an image 1700 captured using the camera 1606 of computing device 1600 of FIG. 72. In this example the image 1700 is an image of an individual's face, including a treatment area, which in this example is the individual's forehead. The captured image 1700 includes indicia 1702 associated with a muscle animation. In this particular example the indicia 1702 are rhytids in the form of a series horizontal forehead lines. As shown in FIG. 74 the horizontal forehead line indicia 1702 in this example is associated with animation of the frontalis muscles 1706.

The computing device 1600 utilizes machine vision or computer vision technologies to process the captured image 1700 and identify the indicia 1702. After identifying the indicia 1702, the computing device 1600 may then identify a pattern of target injection sites appropriate for the particular indicia 1702 identified.

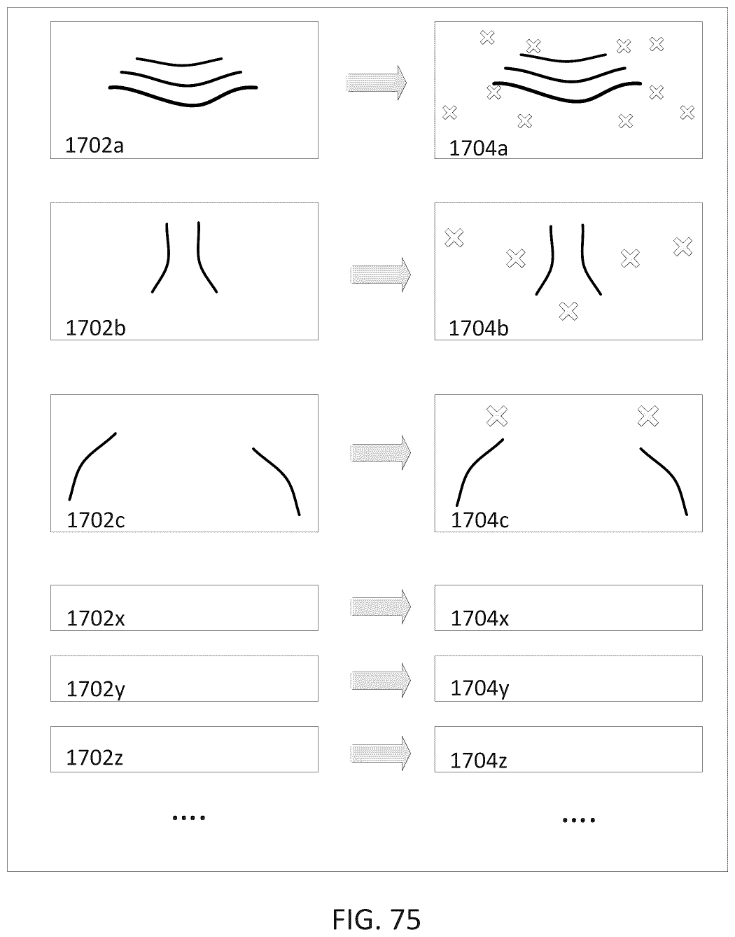

In some implementations the system may use a pre-defined relational data set to identify a pattern of target injection sites appropriate for the particular indicia identified. FIG. 75 schematically represents one example of a database relating different types of indicia 1702a-c to different target medicament injection site patterns 1704a-c. In the example of FIG. 75 the database includes: (1) a specific target medicament injection site pattern 1704a for horizontal forehead line indicia 1702a associated with animation of the frontalis muscles, (2) a specific target medicament injection site pattern 1704b for vertical forehead frown lines indica 1702b associated with animation of the glabellar complex, and (3) a specific target medicament injection site pattern 1704c for nasolabial folds indicia 1702c associated with animation of the levator labii superioris alaequae nasi. These three are just non-limiting examples, and as indicated by boxes for additional indicia 1702x-z and additional injection site patterns 1704x-z, the database may include a variety of injection site patterns 1704 associated with a variety of indicia 1702.

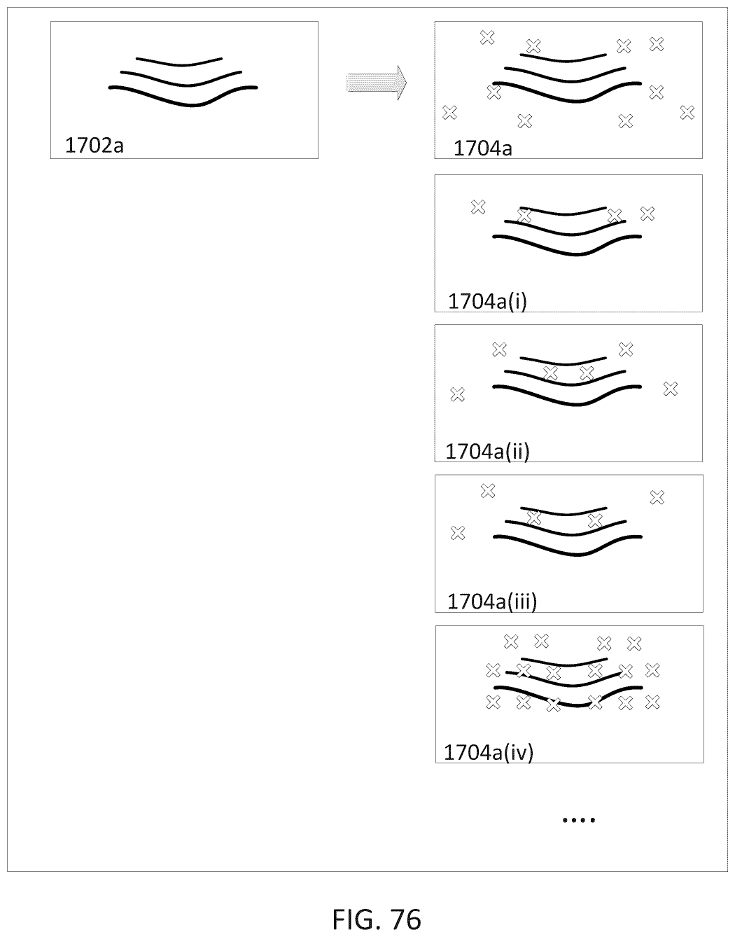

Additionally, while the example database in FIG. 75 shows one injection site pattern 1704 for each type of indicia 1702, in other implementations the database or other relational data set may include multiple injection site patterns for each type of indicia. FIG. 76 shows example of additional injection site patterns 1704a (i)-(iv) that the system may select for horizontal forehead line indicia 1702a. The system may select among the injection site patterns 1704a and 1704a (i)-(iv) based on specific characteristics of the indicia 1702 in the captured image or on other aspects of the captured image (e.g. the indicia's 1702 relative position and/or size to the eyebrows, eyes, nose, or other features in the captured image or based on other aspects).

The system may also determine other aspects of a treatment in addition to determining a particular target injection sites. For example, the system may determine types and or dosages of medicament for each target injection site. As another example, the system may scale, orient, or otherwise alter the selected injection site pattern to best fit the captured image or particular aspects of the captured image.

As noted earlier, in other implementations the system does not use a data base or other pre-defined relational data set for determining targeting data, but instead may use a trained neural network configured to receive as an input an image or other pre-treatment data about a treatment area associated with the person's skin, and output targeting data for one or more target medicament injection sites relative to the treatment area.

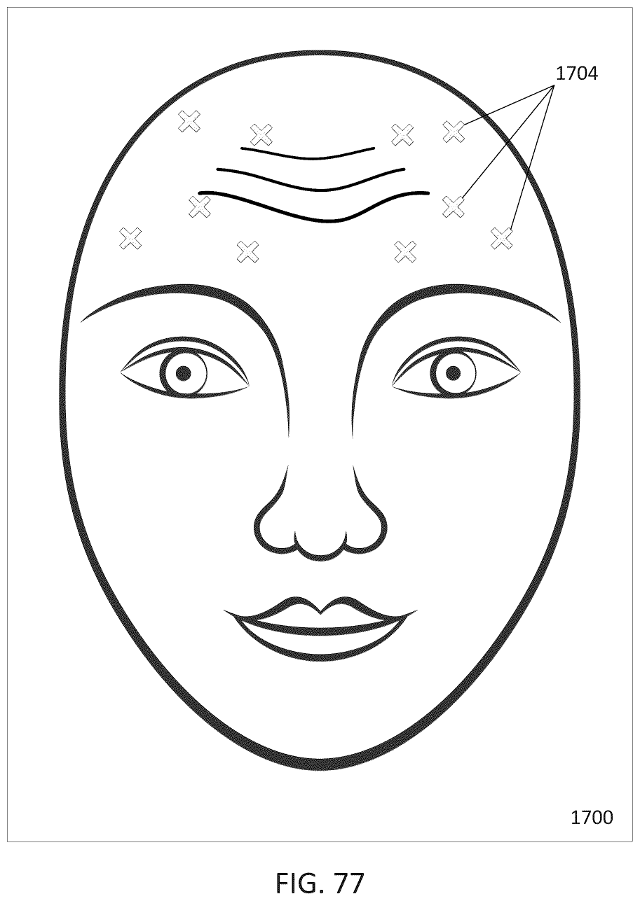

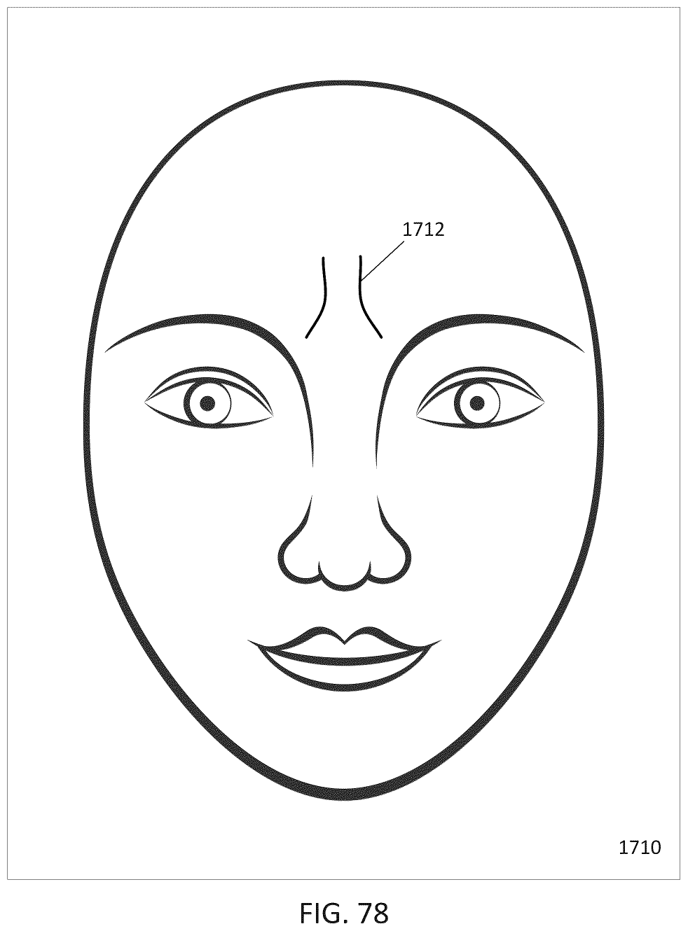

FIG. 77 shows an example of outputted targeting data in the form of an image displayed on display 1608 of computing device 1600, which in this example is the captured image 1700 overlaid with the selected injection site pattern 1704. The image displayed on display 1608 provides guidance to a user for aligning the medicament injection component 1500 with the target medicament sites.

FIGS. 78-80 and 81-83 show additional examples of using a captured image of a treatment area to identify and output targeting data for target medicament injection sites relative to the treatment area.

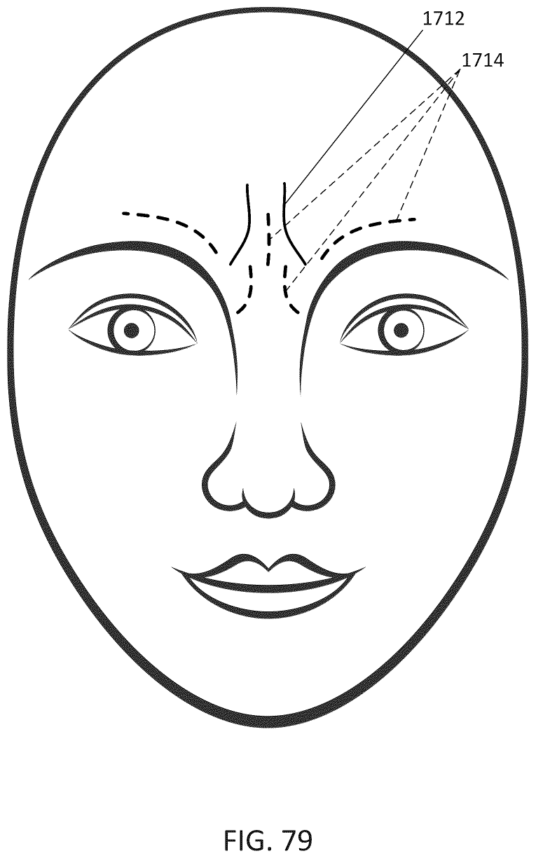

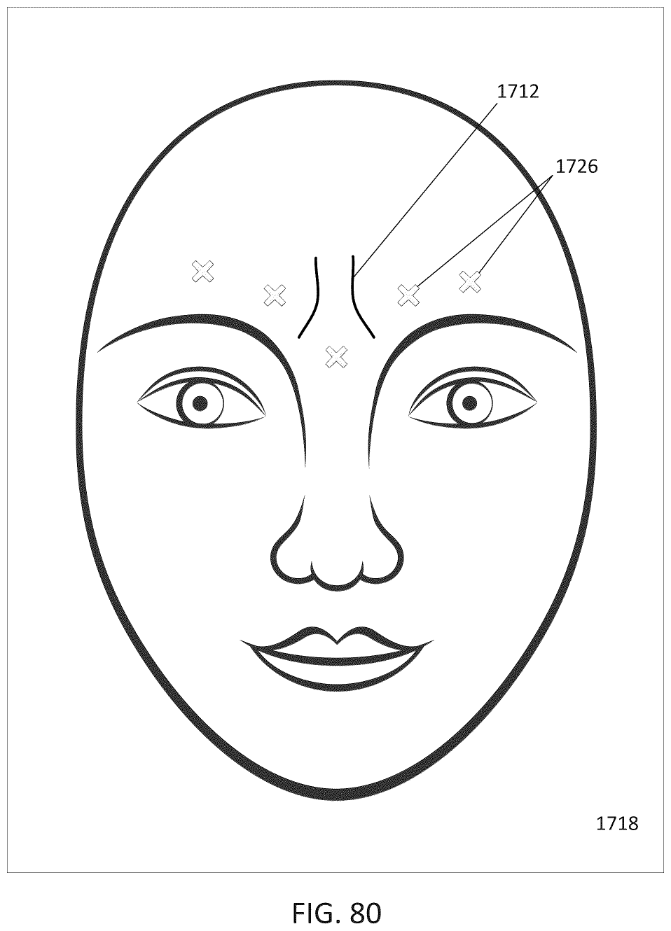

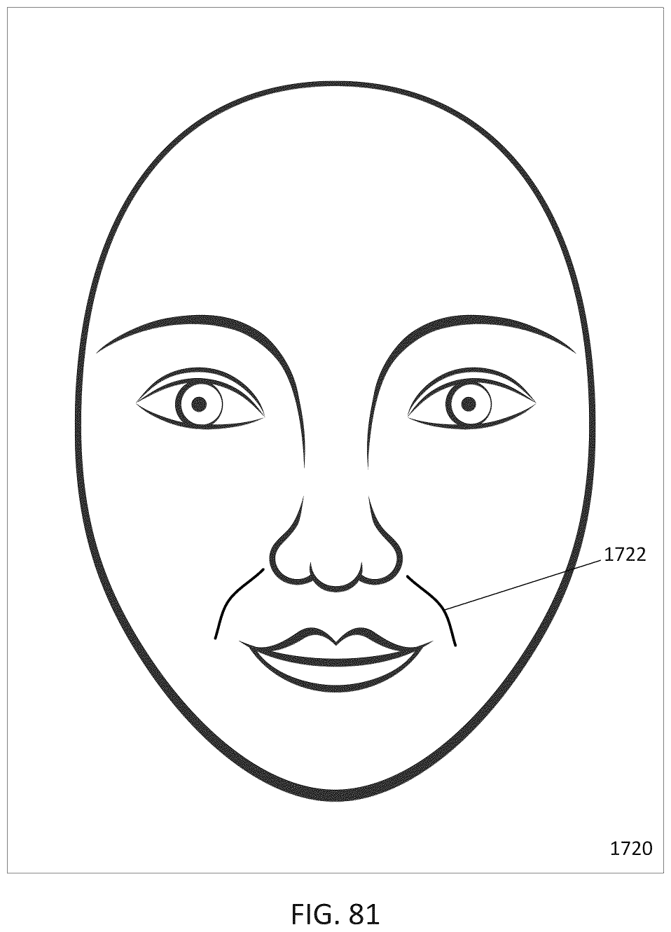

In the example of FIGS. 78-80 the captured image 1710 (FIG. 78) includes indicia 1712 in the form of frown lines, which are associated with animation of the glabellar complex 1714 (FIG. 79). As shown in FIG. 80, based on the captured image 1710 the system may output a target injection site pattern 1716 overlaid on an image 1718 including the treatment area.

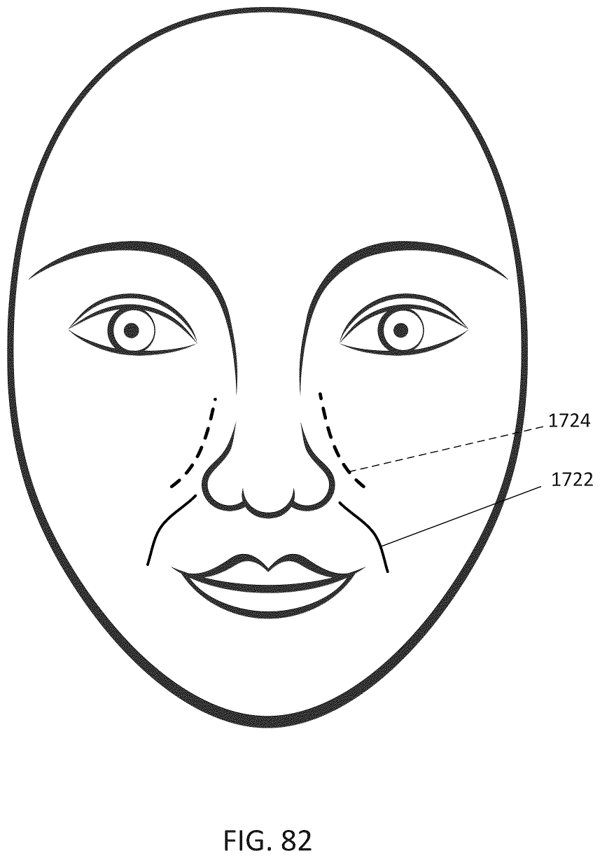

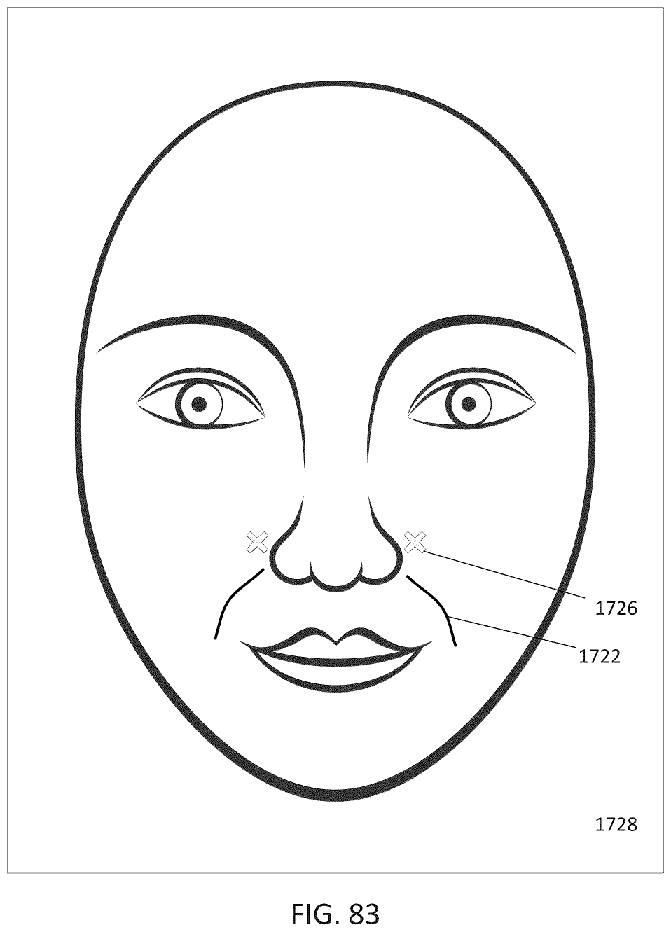

In the example of FIGS. 81-83 the captured image 1720 (FIG. 81) includes indicia 1722 in the form of nasolabial folds, which are associated with animation of the levator labii superioris alaequae nasi 1724 (FIG. 82). As shown in FIG. 83, based on the captured image 1720 the system may output a target injection site pattern 1726 overlaid on an 1728 including the treatment area.

### Post Treatment

Botulinum Toxin and similar medicaments often do not take effect instantaneously, and it may be several days before muscle animation is inhibited to a desired level. In some implementations the system may be configured to receive post-treatment data about the previously treated treatment area(s), and, if necessary, recommend a retreatment. In one example, the system may reperform the process of receiving data about the treatment area (e.g. an image of the treatment area), process the data to identify indicia of undesirable remaining muscle animation, and output targeting data for one or more target medicament injection sites for retreatment. In some implementations the target medicament injection sites for retreatment may be the same injection sites and may be at the same dosages as the earlier treatment. In other implementations the system may recommend a different injection site pattern and/or different injection dosages for the retreatment. In some implementations, the system is configured to retain data about the earlier treatment, and the outputted recommendation for the retreatment may be based on both the received post-treatment data and the data about the earlier treatment.

As with the original treatment, the system may utilize machine vision or computer vision technologies to process a captured post-treatment image and identify indicia in the image associated with a muscle animation to be retreated. The system may utilize artificial intelligence functionality and/or pre-defined relational data sets that are the same or similar to the ones described above for outputting a recommendation for a retreatment.

### Follow-Up Treatments

Botulinum Toxin and similar medicaments will lose their efficacy over time, which may result in a return in undesired muscle animation and the indicia (rhytids) associated with that return of muscle animation. In some implementations the system may be configured to receive follow-up data about previously treated treatment area(s) and recommend follow-up treatments if indicated based on the follow-up data.

In one example, the system may reperform the process of receiving data about the treatment area (e.g. an image of the treatment area), process the data to identify indicia of returning muscle animation, and output targeting data for one or more target medicament injection sites for a follow-up treatment. In some implementations the target medicament injection sites for the follow-up treatment may be the same injection sites and may be at the same dosages as the earlier treatment. In other implementations the system may recommend a different injection site pattern and/or different injection dosages for the follow-up treatment. In these examples, if the system identifies returning or new muscle animation, the system will recommend an appropriate follow-up treatment and if no muscle animation is identified (or no significant muscle animation, such as no muscle animation above a threshold level of animation) is identified, then the system will recommend that no follow-up treatment is necessary at that time.

As with the original treatment, the system may utilize machine vision or computer vision technologies to process a captured image and identify indicia in the image associated with a return of muscle animation. The system may utilize artificial intelligence functionality and/or pre-defined relational data sets that are the same or similar to the ones described above for outputting a recommendation for a follow-up treatment.

In some implementations, the system is configured to retain data about the earlier treatment(s), and the outputted recommendation for the follow-up treatment may take into account retained data about earlier treatment(s). For example, the system may retain data about previous medicament injections facilitated by the system and about the effectiveness of those previous medicament injections. Non-limiting examples of the types of data the system may retain about previous medicament injections includes data about one or more of: (1) number, (2) location, and/or (3) dosage of medicament injections. Non-limiting examples of the types of data the system may retain about the effectiveness of previous medicaments injections includes data about (1) previous medicament injections that successfully inhibited muscle animation (including for instance data about number, location, and/or dosage of those medicament injections) (with success determined, for instance, by analysis of post-treatment data), (2) previous medicament injections that did not successfully inhibit muscle animation (including for instance data about number, location, and/or dosage of those medicament injections) (with lack of success determined, for instance, by analysis of post-treatment data), and (3) effective short term dose titration (i.e. medicament dosage required to successfully inhibit muscle animation), (4) effective long term dose titration (i.e. medicament dosage required to maintain inhibition of muscle animation for a time period).

The retained data about earlier treatments may allow the system to become increasingly customized to its user(s) over time. For instance, in implementations that use a trained neural network or other artificial intelligence functionality for determining aspects of a recommend treatment, retreatment, and/or follow-up treatment, the retained data may be used to alter the neural network or other artificial intelligence functionality over time so that its recommendations are even more tailored to its user(s) over time. As one example, retained data may indicate that a particular user requires higher medicament dosages to successfully inhibit particular muscle animations, and the system may accordingly recommend larger individual dosages and/or additional medicament injections in future recommendations. As another example, retained data may indicate that a particular user experiences relatively short term loss of muscle animation in response to treatments, and the system may accordingly prompt for potential follow-up treatments at greater frequency.

### Example Method of Administering a Botulinum Toxin Treatment

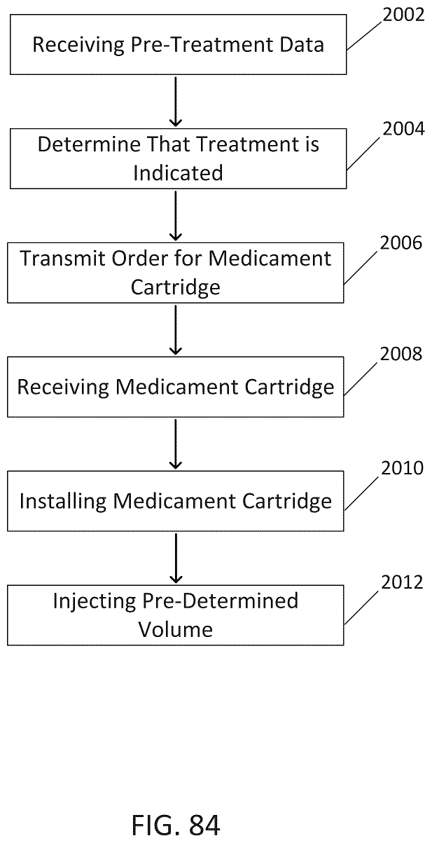

FIG. 84 shows an example of a method of administering a botulinum toxin treatment. In this example the method utilizes a medicament cartridge received from a supplier that contains a constituted botulinum toxin.

In some implementations the constituted botulinum toxin has a ratio in the range of 1 to 100 units of botulinum toxin or 1 to 200 units of botulinum toxin to 0.1 mL to 0.2 mL of dilutent. The dilutent may be a sodium chloride solution, e.g. a 0.9% sodium chloride solution (0.9 grams of sodium chloride per 100 ml of water). The medicament cartridge may contain, in some implementations, between 1 and 10 mL of constituted botulinum toxin.

Prior to use the medicament cartridge may be hermetically sealed and otherwise configured such that it can be stored and transported at a wide range of temperatures, including at ambient temperatures in the range of 10 degrees Celsius to 33 degrees Celsius.

In the method of FIG. 84, the medicament cartridge may be part of a medicament injection system that also includes a medicament injection device. The system (including the cartridge and the device) are configured such that when the medicament cartridge is installed in the medicament injection device, actuation of the medicament injection device injects a pre-determined volume of the constituted and pre-diluted botulinum toxin from the medicament cartridge. In some implementations the medicament injection device may be configured to change the pre-determined volume for injection (e.g. in some procedures the medicament in injection device may need to inject a larger or a smaller dosage volume than for other procedures). In other implementations the medicament injection device may also inject the same pre-determined volume.

The medicament cartridge and medicament injection device may be sized, structured and otherwise configured for replaceable installation (partially or entirely) of the cartridge into the device in the same or similar fashion to the examples described above, such as with cartridges 133, 332, 432, 532, 632, 732. The medicament cartridge may have an outer housing that is dimensions and otherwise configured to be received by a receptacle of the medicament injection device and connect to the medicament injection device such that the device can be actuated to inject a pre-determined volume of liquid from the medicament cartridge. In some implementations the cartridges may be configured to hold a liquid volume of the constituted and diluted botulinum toxin of between 1 and 10 mL. As shown in some of the earlier examples, the cartridge may include an internal plunger that interacts with a piston or other component of the medicament injection device for dispensing medicament from the cartridge. In other implementations the cartridge may be otherwise configured to interact with the medicament injection device for dispensing medicament from the cartridge. In some implementations the medicament cartridge including an injection tip, whereas in other implementations the medicament cartridge includes a nozzle configured to connect to an injection tip of the medicament injection device.

Returning to the method of FIG. 84, at steps 2002 and 2004 the method involves using a computing device of a custom medicament injection system to receive pre-treatment data about a treatment area associated with the person's skin, and, based on the pre-treatment data about the treatment area, determine that a treatment is indicated. In some implementations the custom medicament injection system, including the computing device, may be the same or similar as the custom medicament injection system of FIG. 72 discussed above. In other implementations the custom medicament injection system used in the method of FIG. 84 may be different. In some implementations the pre-treatment data received at step 2002 may be the same or similar to pre-treatment data discussed above (e.g. a captured image of the person's skin) or may take on other forms. In the context of FIG. 84, “pre-treatment” may refer to an initial treatment or a subsequent treatment (e.g. a follow up treatment as described above). In some implementations the presence of certain indicia in a captured image of the person's skin (e.g. indicia 1702 in FIG. 73 discussed above) may determine whether a treatment is indicated. In other implementations, determining whether a treatment is indicated may be done in other ways.

At step 2006 in FIG. 84 the method includes, based on the treatment indication, transmitting an order for a medicament cartridge from a supplier. In some implementations the computing device may be configured to automatically transmit an order for a medicament cartridge based on the treatment indication. In other implementations the user and/or a physician may place the order with the supplier.

At step 2008 in FIG. 84 the method includes receiving the medicament cartridge from the supplier, with the medicament cartridge holding a constituted and pre-diluted botulinum toxin. At step 2010 the medicament cartridge is installed in a medicament injection device.

Finally, at step 2012 in FIG. 84, the medicament injection device is positioned on or adjacent the person's skin and the medicament injection device is actuated to inject a pre-determined volume of liquid from the medicament cartridge into the person.

### Example Medicament Injection Device and Method for Reconstituting Botulinum Toxin

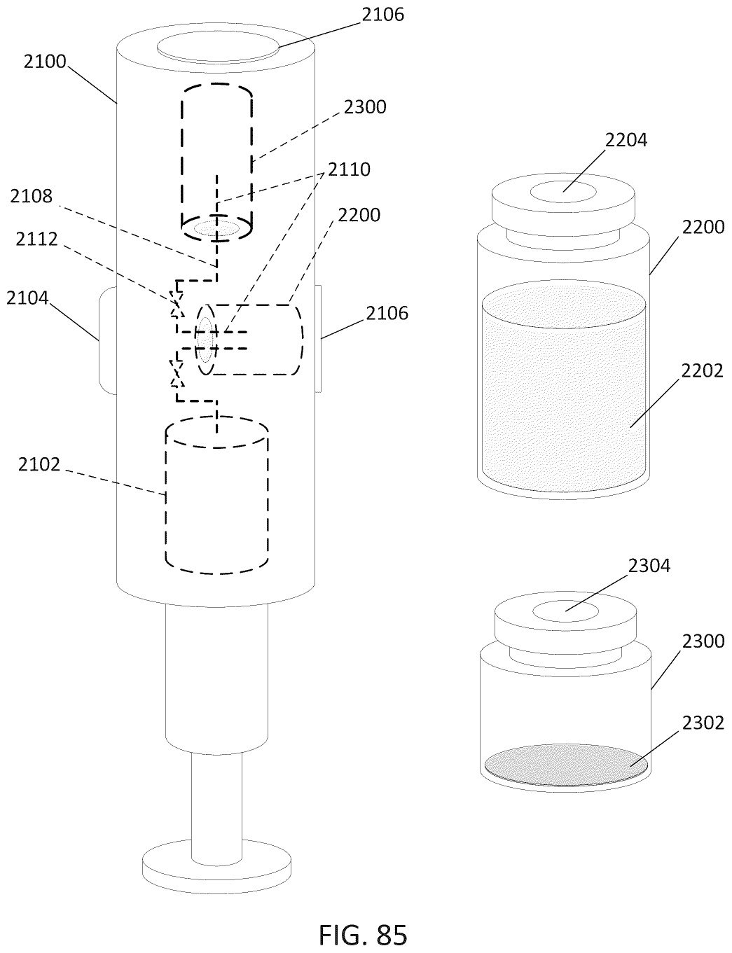

In the example described immediately above the medicament cartridge is received from a supplier in a constituted state. In other implementations the medicament may be reconstituted in the medicament injection device itself. For instance, the medicament injection device may be configured to mix liquid from a liquid supply with medicament from the medicament cartridge prior to injection. One example of this is shown in FIGS. 44-52 described above. Another example is shown schematically in FIG. 85.

FIG. 85 schematically shows an example of a medicament injection device 2100, a medicament cartridge 2200 for removable installation in the medicament injection device 2100, and a liquid supply cartridge 2300 also for removable installation in the medicament injection device 2100.

In this example the medicament cartridge 2200 contains a medicament 2202 that, prior to installation in the medicament injection device 2100, is in a state that has not yet been reconstituted. For instance, the medicament 2202 may be in a dry/dehydrated state such as may be obtained by freeze drying or vacuum drying the medicament. The medicament cartridge 2200 may be in a sealed state. For instance, it may be vacuum sealed. The medicament cartridge 2200 includes a stopper 2204 that may be pierced by a syringe or other piercing tip to inject and/or withdraw liquid into the medicament cartridge 2200.

In one particular implementation, the medicament cartridge is a vacuum sealed vial of botulinum toxin containing in the range of 10 units to 500 units of botulinum toxin per vial. The medicament cartridge may be a standard vial of botulinum toxin that suppliers provide to medical practitioners for botulinum toxin treatments.

In this example the liquid supply cartridge 2300 contains a liquid 2302 for reconstituting the medicament 2202 in the medicament cartridge 2200. Like the medicament cartridge 2200, the liquid supply cartridge 2300 includes a stopper 2304 that may be pierced by a syringe or other piercing tip to withdraw liquid 2303 from the liquid supply cartridge 2300.

In one particular example the liquid supply cartridge 2300 is a vial of sodium chloride solution. The vial of sodium chloride solution may be a standard vial of such solution provided by suppliers to medical practitioners for reconstituting botulinum toxin.

As schematically shown in FIG. 85, the medicament injection device 2100 includes a medicament injector 2102 which may be configured similarly to other medicament injectors described above. The medicament injection device 2100 also includes an injection actuator 2104 that operates the medicament injector 2102 to inject a pre-determined volume of reconstituted medicament from the medicament injection device 2100 drawn from the medicament cartridge 2200 that is removably installed in the device 2100.

In this example the medicament cartridge 2200 and liquid supply cartridge 2300 may be removably installed in the medicament injection device 2100. As schematically shown in FIG. 85, the medicament injection device 2100 includes access features 2106 (e.g. openable covers) for allowing installation of the cartridges 2200, 2300 in the device 2100.

Upon installation of the cartridges 2200, 2300 in the device, medicament cartridge 2200 may become in fluid communication with liquid supply cartridge 2300 and also with medicament injector 2102. In this particular example medicament cartridge 2200 is in fluid communication with liquid supply cartridge 2300 via liquid supply 2108, which may be a fluid conduit extending between and fluidly connecting medicament cartridge 2200 and liquid supply cartridge 2300. The liquid supply 2108 fluid conduit may include ports 2110 with piercing tips that pierce the stoppers 2204, 2304 of the cartridges 2200, 2300 when they are installed in the medicament injection device 2100. Ports 2110 may be replaceable components of the medicament injection device 2100.

Upon installation of the cartridges 2200, 2300 in the device 2100, the device 2100 may cause a pre-determined volume of liquid to flow through liquid supply 2108 from the liquid supply cartridge 2300 into the medicament cartridge 2200 thereby reconstituting the medicament 2302. Valve 2112 may control the volume of liquid supplied to the medicament cartridge 2200. In some implementations the medicament injection device 2100 flows a predetermined volume of sodium chloride solution into the medicament cartridge 2200 to reconstitute the botulinum toxin at a ratio in the range of 1 to 200 units of botulinum toxin to 0.1 mL to 0.2 mL of sodium chloride solution.

Upon installation of the medicament cartridge 2200 in the device 2100, cartridge 2200 may become in fluid communication with the medicament injector 2102 in a similar fashion to that described above for the fluid communication between the medicament cartridge 2200 and the liquid supply cartridge 2300.

While the preferred embodiments of the invention have been described above, it will be recognized and understood that various modifications may be made in the invention and the appended claims are intended to cover all such modifications which may fall within the spirit and scope of the invention.

1. A custom medicament injection system comprising:
(a) a medicament injection component comprising an injector and a replaceable medicament cartridge, wherein actuation of the injector injects a set medicament dosage into or beneath a person's skin;
(b) a computing device comprising:
(i) a processor device; and
(ii) a non-transitory computer-readable medium having instructions stored thereon that are executable by the processor device to cause the computing device to:
(1) receive pre-treatment data about a treatment area associated with the person's skin;
(2) based on the pre-treatment data about the treatment area, identify one or more target medicament injection sites relative to the treatment area; and
(3) output targeting data for the one or more target medicament injection sites relative to the treatment area.

2. The custom medicament injection system of claim 1 wherein identifying the one or more target medicament injection sites relative to the treatment area comprises identifying at least one muscle animation associated with the person's skin.

3. The custom medicament injection system of claim 1 wherein the pre-treatment data is at least one captured image of the treatment area including at least one indicia associated with at least one muscle animation.

4. The custom medicament injection system of claim 3 wherein the at least one captured image is a static image or a video image.

5. The custom medicament injection system of claim 3 wherein the at least one indicia is at least one rhytid in the treatment area.

6. The custom medicament injection system of claim 5 wherein the non-transitory computer-readable medium has instructions stored thereon that are executable by the processor device to cause the computing device to, based on a spatial relationship between the at least one rhytid and an animation of at least one muscle associated with the at least one rhytid, identify the one or more target medicament injection sites relative to the treatment area.

7. The custom medicament injection system of claim 3 wherein the computing device component further comprises a camera that captures the at least one image.

8. The custom medicament injection system of claim 1 wherein the system further comprises a display.

9. The custom medicament injection system of claim 7 wherein the display outputs the targeting data.

10. The custom medicament injection system of claim 8 wherein the display outputs the targeting data overlaid on an image comprising the treatment area.

11. The custom medicament injection system of claim 9 wherein the computing device component comprises the display.

12. The custom medicament injection system of claim 1 wherein the non-transitory computer-readable medium has instructions stored thereon that are executable by the processor device to cause the computing device to identify one or more target medicament dosages for the one or more target medicament injection sites.

13. The custom medicament injection system of claim 1 wherein the non-transitory computer-readable medium has instructions stored thereon that are executable by the processor device to cause the computing device to receive post-treatment data about the treatment area associated with the person's skin.

14. The custom medicament injection system of claim 13 wherein the non-transitory computer-readable medium has instructions stored thereon that are executable by the processor device to cause the computing device to, based on the post-treatment data, output a recommendation for a retreatment.

15. The custom medicament injection system of claim 14 wherein the retreatment includes at least one modified target medicament injection site or at least one modified injection dosage.

16. The custom medicament injection system of claim 13 wherein the non-transitory computer-readable medium has instructions stored thereon that are executable by the processor device to cause the computing device to receive follow-up data about the treatment area associated with the person's skin.

17. The custom medicament injection system of claim 16 wherein the non-transitory computer-readable medium has instructions stored thereon that are executable by the processor device to cause the computing device to retain data about earlier treatments.

18. The custom medicament injection system of claim 17 wherein the non-transitory computer-readable medium has instructions stored thereon that are executable by the processor device to cause the computing device to, based on the data about earlier treatments and based on the follow-up data, output a recommendation for a follow-up treatment.

19. The custom medicament injection system of claim 18 wherein the follow-up treatment includes at least one modified target medicament injection site or at least one modified injection dosage.

20. A method of administering a botulinum toxin treatment, the method comprising:
(a) receiving a medicament cartridge from a supplier, the medicament cartridge comprising a constituted botulinum toxin;
(b) installing the medicament cartridge in a medicament injection device, wherein the medicament injection device and medicament cartridge are part of a medicament injection system configured such that, when the medicament cartridge is installed in the medicament injection device, actuation of the medicament injection device injects a pre-determined volume of the constituted botulinum toxin from the medicament cartridge; and
(c) positioning the medicament injection device on or adjacent a person's skin and actuating the medicament injection device to inject the pre-determined volume of liquid from the medicament cartridge into the person.

21. The method of claim 20 wherein the method of administering the botulinum toxin treatment is a method of self-administering the botulinum toxin treatment.

22. The method of claim 21 wherein the constituted botulinum toxin comprises a ratio in the range of 1 to 200 units of botulinum toxin to 0.1 mL to 0.2 mL of diluent.

23. The method of claim 22 wherein the diluent is a sodium chloride solution.

24. The method of claim 22 wherein the cartridge comprises a liquid volume of the botulinum toxin and diluent of between 1 and 10 mL.

25. The method of claim 20 wherein the medicament cartridge is received in a sealed and ambient temperature state.

26. The method of claim 20 wherein the medicament cartridge comprises an internal plunger.

27. The method of claim 20 wherein the medicament cartridge comprises an injection tip or comprises a nozzle configured to connect to an injection tip of the medicament injection device.

28. The method of claim 20 wherein the medicament cartridge comprises an outer housing, and wherein the medicament cartridge device comprises a medicament receptacle dimensioned and configured to receive at least a portion of and connect to the outer housing of the medicament cartridge such that the medicament injection device can be actuated to inject the pre-determined volume of liquid from the medicament cartridge.

29. The method of claim 20 wherein the method further comprises, prior to receiving the medicament cartridge, using a computing device of a custom medicament injection system to receive pre-treatment data about a treatment area associated with the person's skin, and, based on the pre-treatment data about the treatment area, determine that a treatment is indicated.

30. The method of claim 29 wherein the method further comprises, based on the treatment indication, transmitting an order for the medicament cartridge from the supplier.

31. A medicament injection device comprising:
(a) a medicament injector;
(b) an injection actuator that operates the medicament injector to inject a pre-determined volume of a medicament from the medicament injection device, the medicament drawn from a medicament cartridge removably installed in the medicament injection device;
(c) a liquid supply in fluid communication with the medicament cartridge when the medicament cartridge is removably installed in the medicament injection device, the medicament injection device mixing liquid from the liquid supply with the medicament from the medicament cartridge prior to injection.

32. The medicament injection device of claim 31 wherein the medicament injection device flows a pre-determined volume of liquid through the liquid supply into the medicament cartridge removably installed in the medicament injection device.

33. The medicament injection device of claim 32 wherein the liquid supply flows the pre-determined volume of liquid into the medicament cartridge from a liquid supply cartridge removably installed in the medicament injection device.

34. The medicament injection device of claim 33 wherein the medicament cartridge comprises a vial of botulinum toxin and wherein the liquid supply cartridge comprise a vial of sodium chloride solution.

35. The medicament injection device of claim 33 wherein the medicament injection device comprises a first port with a first piercing tip for piercing a stopper of the medicament cartridge and a second port with a second piercing tip for piercing a stopper of the liquid supply cartridge when the medicament and liquid supply cartridges are removably installed in the medicament injection device.

36. The medicament injection device of claim 35 wherein the first and second ports comprise replaceable components of the medicament injection device.

37. A method of administering a medicament treatment using a medicament injection device comprising a medicament injector and an injection actuator that operates the medicament injector to inject a pre-determined volume of a medicament from the medicament injection device, the method comprising:
(a) installing a medicament cartridge into the medicament injection device, the medicament cartridge comprising a medicament;
(b) after the medicament cartridge is installed into the medicament injection device, supplying a liquid into the medicament cartridge to reconstitute the medicament; and
(c) after reconstituting the medicament, operating the injection actuator to inject a pre-determined volume of the reconstituted medicament from the medicament injection device.

38. The method of claim 37 further comprising installing a liquid supply cartridge into the medicament injection device, the liquid supply cartridge comprising the liquid, and wherein supplying the liquid into the medicament cartridge comprises flowing the liquid from the liquid supply cartridge into the medicament cartridge.

39. The method of claim 38 wherein the medicament cartridge comprises a vial of botulinum toxin and wherein the liquid supply cartridge comprise a vial of sodium chloride solution.

40. The method of claim 39 wherein the vials of botulinum toxin and sodium chloride solution are installed in the medicament injection device in a removable fashion.

41. The method of claim 39 wherein the medicament injection device flows a predetermined volume of sodium chloride solution into the botulinum toxin vial to reconstitute the botulinum toxin at a ratio in the range of 1 to 200 units of botulinum toxin to 0.1 ml to 0.2 mL of sodium chloride solution.

42. The method of claim 37 wherein the medicament cartridge comprises a vacuum sealed vial of botulinum toxin.

43. The method of claim 37 wherein the medicament cartridge comprises a vacuum sealed vial of dehydrated botulinum toxin.
